(19) United States
(12) Patent Application Publication
(10) Pub. No.: US 2026/0015619 A1
(43) Pub. Date: Jan. 15, 2026

(54) METHODS FOR PREPARING APTAMERS FOR SMALL MOLECULES

(71) Applicant: THE TRUSTEES OF COLUMBIA UNIVERSITY IN THE CITY OF NEW YORK, New York, NY (US)

(72) Inventors: Milan STOJANOVIC, New York, NY (US); Kyungae YANG, New York, NY (US)

(21) Appl. No.: 19/331,343

(22) Filed: Sep. 17, 2025

Related U.S. Application Data
(63) Continuation of application No. PCT/US2024/020210, filed on Mar. 15, 2024.
(60) Provisional application No. 63/490,967, filed on Mar. 17, 2023.
(60) Provisional application No. 63/492,958, filed on Mar. 29, 2023.

(51) Int. Cl. C12N 15/115; A61K 31/7088; C40B 30/10; C40B 40/06; C40B 50/00

(52) U.S. Cl. CPC C12N 15/115; A61K 31/7088; C40B 30/10; C40B 40/06; C40B 50/00; C12N 2310/16

(57) ABSTRACT

The present disclosure provides, inter alia, a novel, functional group-guided method for preparing aptamers for small molecules, including those that cannot be obtained by standard protocols. Also provided are aptamers identified and prepared using the method disclosed herein, compositions comprising such aptamers, and methods and kits for treating a condition using the aptamers and/or compositions disclosed herein.

### CROSS REFERENCE TO RELATED APPLICATIONS

The present application is a continuation of PCT international application no. PCT/US2024/020210, filed on Mar. 15, 2024, which claims benefit of U.S. Provisional Patent Application Ser. No. 63/490,967, filed on Mar. 17, 2023, and U.S. Provisional Patent Application Ser. No. 63/492,958, filed on Mar. 29, 2023. The contents of above applications are incorporated by reference herein in their entireties.

### GOVERNMENT FUNDING

This invention was made with government support under HDTRA1-16-1-0053 awarded by the Defense Threat Reduction Agency, DK126739, GM138843 and DA045550 awarded by the National Institutes of Health, and 1518715 and 1763632 awarded by the National Science Foundation. The government has certain rights in the invention.

### FIELD OF DISCLOSURE

The present disclosure provides, inter alia, a novel, functional group-guided method for preparing aptamers for small molecules, including those that cannot be obtained by standard protocols.

### INCORPORATION BY REFERENCE OF SEQUENCE LISTING

This application contains references to amino acids and/or nucleic acid sequences that have been filed concurrently herewith as sequence listing XML file “CU23271-PCT-seq.xml”, file size of 147,925 bytes, created on Mar. 14, 2024. The aforementioned sequence listing is hereby incorporated by reference in its entirety pursuant to 37 C.F.R. § 1.52(e)(5).

### BACKGROUND OF THE DISCLOSURE

Aptamers are oligonucleotide-based receptors isolated from random libraries through cycles of enrichment based on target affinity coupled to amplifications (1-4). Aptamers can be selected for a variety of small molecules for which antibodies cannot, that is, targets ignored by the immune system even when conjugated to carrier proteins, e.g., neurotransmitters (5) and amino acids (6, 7). Once available, aptamers can be readily engineered into various sensor formats (3,4), including for use as fluorescent (8), electrochemical (9), or electronic biosensors (10).

One of the main obstacles to the broad application of aptamers in biosensing is a lack of aptamers with appropriate affinities for many important low molecular weight targets (3, 4). For example, there is currently no isolated DNA aptamers for two clinically important molecules, the amino acid leucine and the antifungal agent voriconazole. This disclosure is directed to meeting these and other needs.

### SUMMARY OF THE DISCLOSURE

Aptameric receptors are important biosensor components, yet our ability to identify them depends on the target structures. The present disclosure analyzed the contributions of individual functional groups on small molecules to binding within 27 target-aptamer pairs, identifying potential hindrances to receptor isolation, for example, negative cooperativity between sterically hindered functional groups. To increase the probability of aptamer isolation for important targets, such as leucine and voriconazole, where multiple previous selection attempts failed, the present disclosure designed tailored strategies focused on overcoming individual structural barriers to successful selections. This approach enables us to move beyond standardized protocols into functional group-guided searches, relying on sequences common to receptors for targets and their analogs to serve as anchors in regions of vast oligonucleotide spaces wherein useful reagents are likely to be found.

By identifying specific structural factors that contributed to unsuccessful selections, the present disclosure provides a functional group-guided approach to isolate aptamers that were previously unattainable. Each target that cannot be adequately addressed through standard selection protocols, can be now viewed as a candidate for the functional group-guided approach. Individual steps developed in the present disclosure include insertion reselection, carrying-over and anchoring of partial motifs, the use of metal complexes as “protective” groups, placeholders, cross-linkers, and the synthesis of simpler analogs designed to overcome steric barriers. The novel approach disclosed herein can be further combined with traditional protocols, organic receptor cofactors, and modified bases, while taking into account library designs, to enable isolation of high-quality aptamers and engineering of biosensors for previously inaccessible targets.

Accordingly, one embodiment of the present disclosure is a method for preparing an aptamer for a target molecule. This method comprises the steps of: (a) screening and selecting a candidate aptamer for the target molecule through standard selection protocols; (b) modifying the candidate aptamer by having an N₂₂ random insertion to form a library used to identify a sequence that recognizes at least one functional group on the target molecule; (c) generating a library of random 22-mers with the sequence identified in step (b) positioned next to the closing stem of the candidate aptamer; (d) performing a functional-group binding free energy analysis for aptamers from the library generated in step (c); and (e) identifying the aptamer that has the highest affinity for the target molecule.

Another embodiment of the present disclosure is an aptamer disclosed herein, including those identified by the method disclosed herein.

Still another embodiment of the present disclosure is a composition, including pharmaceutical compositions, comprising one or more aptamer disclosed herein, including those identified and prepared by the method disclosed herein.

A further embodiment of the present disclosure is a method for treating or ameliorating the effects of a condition in a subject in need thereof. This method comprises administering to the subject an effective amount of one or more aptamer disclosed herein, including those identified and prepared by the method disclosed herein or the composition disclosed herein.

Yet another embodiment of the present disclosure is a kit for treating or ameliorating the effects of a condition in a subject in need thereof, comprising an effective amount of one or more aptamer disclosed herein, including those identified and prepared by the method disclosed herein or the composition disclosed herein, packaged with its instructions for use.

### BRIEF DESCRIPTION OF THE DRAWINGS

To facilitate further description of the embodiments of this disclosure, the following drawings are provided to illustrate and not to limit the scope of the disclosure.

FIGS. 1A-1E show the target functional-group binding free energy analysis for aptamers from stem-loop libraries: (FIG. 1A) Using standard protocol, we were unable to isolate aptamers for leucine (1) and voriconazole (2), which share congested carbons (*). (FIG. 1B) Aptamer selections driven by small-molecule-induced stem closures: An oligonucleotide library with a random loop (N₃₆) is hybridized to the complement (capture strand) of a PCR primer. The capture strand is tethered to a column. The column is exposed to target solutions. Sequences that bind the targets and undergo stem stabilization are released, preferentially amplified, and used in the next selection cycle. (FIG. 1C) We measured apparent ^{app}K_{D} values for aptamers and from these calculated the free energies of displacement, ΔG_{D}, based on a fluorescent displacement assay associated with the equilibrium between an aptamer (labeled with fluorescein, F) and a complementary oligonucleotide used for capture in selection (labeled with a quencher, dabcyl, D). Target leads to concentration-dependent increases in fluorescence via equilibria shown. K_{X} and K_{A} and are dissociation constants for a target-aptamer complex without competitor and an aptamer-competitor complex without target, respectively. (FIG. 1D) We isolated the contributions of individual functional groups by subtracting individual ΔG_{D} values of aptamer-target pairs, with these values corrected to account for differences in oligonucleotide quenching. Here, the two targets, methylamine (3) and phenylethylamine (4), differ by a benzyl group. The difference in free energy associated with benzyl group addition is ΔΔG_{GBE} (benzyl). Two aptamers used for this calculation are shown. (FIG. 1E) Cooperativity is assessed by double functional group replacement cycles (18). The ^{app}K_{D} and ΔG_{D} (normalized to average impact of oligonucleotide on equilibrium, in kJ/mol) values are shown next to the targets, with ΔΔG_{GBE} values shown next to the fragments. A ΔΔG_{GBE}>0 indicates a decrease in affinity upon adding a functional group, the ΔG_{C} value (+8.0 kJ/mol) represents the difference between adding functional groups separately (upper horizontal and left vertical values) vs. at the same time (diagonally), which is interpreted as negative cooperativity when both benzyl group and a carboxylate are together present in a molecule.

FIGS. 2A-2E show the analysis of ΔG_{D} and ΔΔG_{GBE} from a set of 27 aptamers: (FIG. 2A) Exemplary targets used to characterize binding optimization to hydrophobic surfaces during selections; hydrophobic/aromatic fragments are shown as brown squares (amines) or green diamonds (amino acids). (FIG. 2B) Regression analysis of target ΔG_{D} vs. # of heavy atoms (other than hydrogen) in aromatic hydrophobic fragments within targets. The regression line including methylamine and two aromatic amines (3, 4, 8, larger brown squares) was used to estimate the contributions of the hydrophobic surfaces in two aromatic amino acids (6, 10) and a non-aromatic hydrophobic amine related to leucine (7). Data for the four aptamers for 6 are shown individually. Methylene blue (9) is the target with the highest affinity for the aptamers isolated directly from N₃₆ libraries. Two amides have ΔG_{D} values above, carboxylates below (diamonds), and histamine (10) and serotonin (11) on the regression line. (FIG. 2C) Additivity of ΔΔG_{GBE} in similar compounds (cf. FIG. 18A-18B): Using the average ΔΔG_{GBE} values of a pair of planar indole-methylene containing molecules and five carboxamides, we estimated □G_{B} for the melatonin (13) aptamer. (FIG. 2D) Distributions of ΔΔG_{GBE} contributions of selected functional groups, for carboxylates (open diamonds, we show position of phenylalanine, 6), carboxamides (circles), guanidiniums (triangles), and hydrophobic groups (squares). We show rounded averages (lines) and standard deviations in kJ/mol. (FIG. 2E) Cooperativity (ΔG_{C}) was assessed through double functional group replacement cycles (FIG. 1E), and we show groups added to methylamine together with carboxylates to obtain individual amino acids (FIG. 17). All data points in FIGS. 2B-2E are results of individual selection experiments, and the uncertainty of this approach can be assessed by four aptamers for phenylalanine, 6, in FIG. 2B, which were isolated in four independent selections.

FIGS. 3A-3F shows the multistep functional group-guided approach to high-affinity aptamers for leucine: (FIG. 3A) Leucine was broken into two fragments, i.e., isobutyl and 2-aminoethanoate. We designed a selection to isolate aptamers sequentially to recognize one, then both fragments, thereby reducing the target-related barriers in each step and increasing the probability of finding leucine aptamers. Shown are the complex of leucine and Cp*Rh(III), 14, and other amino acids for which we observed challenging aptamer cross-reactivities (15, 16). (FIG. 3B) We started with a Cp*Rh(III) aptamer, performing an N₂₂ random insertion to form a library used to identify the iBu.1 sequence, which recognized the isobutyl group. We used a second N₂₂ library to focus selection pressure on the 2-aminoethoanoate group to arrive at leucine aptamers. (FIG. 3C) Secondary structures of the related CpLeu1.0, Leu2.1 (minimized from Leu2.0, FIG. 16C) and CuLeu1.0. The inserted iBu1 motif is shown in gray in CpLeu1.0 and carried-over sections of the CpRh1.0 aptamer are shown in black. The CpLeu1.0 aptamer binds Leu in the presence of Cp*Rh(III), while Leu2.1 binds leucine on its own. The CuLeu1.0 aptamer binds Leu in the presence of Cu(II) with high affinity (^{app}K_{D}˜170 nM). (FIG. 3D) Double-functional group replacement cycle, methylamine (3) to leucine (1). (FIG. 3E) Fluorescence vs. target concentrations in the presence of 40 μM Cu(II) (displacement assay, cf. FIG. 1C) for CuLeu1.0 and four branched-chain amino acids. (FIG. 3F) Preliminary analytical assessment of CuLeu1.0 in mock human sera samples spiked with branched-chain amino acids to mimic values for patients with maple sugar urine disease (MSUD) (FIG. 23A, left). The correlation is between measured values (dilution 1:500, 100 μM Cu(II)) of X′Le (the sensor responsive fraction) vs. added values for {[Leu]+0.57*[allo-Ile]}. The high correlation indicates that this sensor is suitable component of a minimal cross-reactive array for monitoring in patients, although dilution might have to be adjusted depending on targeted range. Allo-Ile is negligible at birth and during the first several postpartum days (11), thus, we show also correlation in the same mock samples but without allo-Ile (circles). Measurements in FIG. 3E and FIG. 3F are in triplicates with SDs shown.

FIGS. 4A-4B show the selection of voriconazole aptamers using an analog: (FIG. 4A) Structure of voriconazole (2) with three fragments (I-III) and its analog 2a with only two (I, II). Voriconazole has one tertiary and one quaternary sp³ carbon. The arrows indicate the perspective used to produce the Newman projections below. The anti(I, III) conformation is similar to an observed crystal structure (29). The voriconazole analog 2a simplifies the largest fragment (III) and was designed for reduced complexity and as a more suitable target for selection. Here, the anti (I, II) conformation is likely to be favored and the dominant epitope in selection. (FIG. 4B) The aptamer Vor1.0 was isolated in the selection protocol that used 2 and 2a in parallel. The secondary structure of Vor1.0 is shown as predicted by mFold (top) and as an alternative secondary structure (bottom), which was subsequently confirmed to be the active sensor structure. Structure-switching allows this aptamer to be captured on the column (i.e., the upper structure allows capture) during the initial stages of selection (cf. FIGS. 27A-27B). A variant of Vor1.0, Vor1.1.4 (which cannot be captured on the column and, thus, was not isolated during selection) was turned into a quenching-FRET sensor and responded to both 2 and 2.a. Using fluorescence, this sensor detected voriconazole concentrations as low as 3 μM; thus, this oligonucleotide is a candidate for incorporation of electrochemical sensors for in vivo monitoring (12).

FIG. 5 shows a schematic of NGS sample preparation as described in Example 1.

FIG. 6 shows the procedure of Thioflavin T assay (left, 96 wells; right 384 wells) as described in Example 1.

FIG. 7A shows the displacement assay as described in Example 1. FIG. 7B shows the results of Aptamer with fluorescein (A) and no quencher. (Left) Receptor/sensor (R) with target (X) but no complementary oligonucleotide (A). These data show that targets have minimal impact on aptamer fluorescence by themselves and can be largely neglected. The results are for all targets, 0-100, relative concentration on x-axis. (Right) The exception is Methylene blue. Here, we see substantial quenching that needs to be accounted for.

FIG. 8 shows the NMR analysis of analog 2a.

FIG. 9 shows the HPLC chromatogram of analog 2a.

FIG. 10 shows the mass spectrum of analog 2a.

FIG. 11 shows a schematic of the black box approach as described in Example 2.

FIG. 12 shows the quenching curve of a leucine aptamer as described in Example 2.

FIG. 13 shows the displacement curve of a leucine aptamer as described in Example 2.

FIG. 14 shows how all the values in the displacement assay are determined.

FIG. 15 shows the mean ITC binding curves (fit to single site to obtain K_{D} values) and representative heat curves (insets) for targets from three different target classes (amino acids (PHE), amines (HIS), and α-amino amides (TYRA)). The binding curves for tyrosinamide showed reproducible evidence of low- and high-affinity aptamer binding sites. The dissociation constants (K_{D}) determined by ITC vs. displacement assay (competitive model used) were similar for phenylalanine and histamine but differed greatly for tyrosinamide possibly due to the poor fit when using a one-site binding model. Data used to fit curves are means±standard errors of the means for N=3 experiments run on different days.

FIG. 16A depicts the data format for all aptamers tested in the present disclosure. FIG. 16C shows how the fitting parameters are reported. FIG. 16C shows the data of all aptamers tested. FIG. 16D shows additional characterization of CuLeu1.0 aptamer: (Left), ThT displacement for Cu(II) only in comparison to Cu(II)+Leu. The x-axis is target, either Cu(II) only, or Cu(II), in the presence of constant Leu (100 μM; shows no displacement) or Leu in the presence of constant Cu(II) (40 μM); (Right) ThT dye displacement of Leu (only), in the absence of Cu(II). It also shows selectivity over lie. Of note, a high concentration of Leu is necessary to displace ThT.

FIG. 17 shows group binding energies from aptamer*ligand pairs and double functional group replacement cycles.

FIG. 18A shows the values used to demonstrate additivity of group binding energies. FIG. 18B shows the calculations of the group binding energies.

FIG. 19 shows the selectivity of Leu2.1.

FIG. 20 shows the support for AAGA as “compatible” sequence, rather than absolutely necessary insertion reselection to eliminate Cu(II) binding site, also generates aptamers related to Leu2.1.

FIG. 21 shows the direct selection for Leu aptamers using Cp*Rh(III) cofactor. Results of earlier attempts to perform direct selections using only Cp*Rh(III) complex as a cofactor. The first three related sequences were identified as responsive to Leu, but preferred Phe. All three were tested because hydrophobic pocket binding to side chain might be slightly adjusted. The last sequence was isolated after a counterselection with Phe; nevertheless, cross-reactivity persisted.

FIG. 22 shows that Newman projections explain specificity through a binding pocket model. The upper section shows the fit between a hypothetical binding pocket and the Leu side chain, while the lower section show other amino acids. Ile shows steric hindrance, which prevents binding, while allo-Ile fits, although with a smaller contact area.

FIG. 23A shows the mock sample formulations for X′Le studies (left) and the sample evaluation (right): These evaluations are done strictly as preliminary screening, with sensors coming as they are from selections, with no further optimizations. Here we show a typical calibration curve at dilutions of 1:500, which is optimal for 75-600 μM range. The midpoint of the “pseudo-linear” range could be adjusted by either dilution or mutagenesis to reduce affinity of the aptamer. For cross-reactive arrays, for example, both quenching and release would be improved by systematic studies of aptamer and capture oligonucleotide to increase signal. All samples were made in stripped plasma. Numbers for real patient samples were provided by Karlla Brigatti and Kevin Strauss, Clinic for Special Children, Strasburg, PA, through mediation of the MSUD Family Group, to which we added also a second set with similar numbers. Then, identical samples were also made, but without allo-lie. FIG. 23B shows expanded measurements on mock samples: Here, we show the follow up studies, now over two different days, of ability of CuLeu1.0 to measure X′Le and Leu concentrations, using mock samples with (left) and without (right) allo-lie. We see that it would be beneficial to increase dilution to improve measurements at higher X′Le values. As is logical, the parallel Leu measurements are better fit with higher precision sensor range. Interestingly, even higher concentrations of Ile and Val do not have much of an impact. All measurements are in triplicates.

FIG. 24A shows the multistep selection leading to lie aptamer. (A) The insertion library based on the CpRh 1.0 aptamer backbone uses the complex as a placeholder for 2-aminoetanoate. (B) The isolated Ile-Cp*Rh(iii) aptamer (CpRhIIe-apt) using insertion-reselection, (C) Re-randomized library while using Ile-binding sequence to anchor our search. (D) The isolated aptamer (CuIle1.1_apt) recognizing Ile-Cu(ii) complex from the library per (C). FIG. 24B shows the data for CpRhIIE-apt and CuIle-apt.

FIG. 25 shows the direct and two step selections of Glu aptamers with Cp*Rh(III) cofactor. On the left, we have an example of an aptamer isolated directly from a Cp*Rh(III)*Glu selection, chosen for this demonstration. On the right is its analog from the two-step selection. These aptamers have substantial sequence overlap in sections that recognize Glu-but only in coordination with Cp*Rh(III). Subsequent attempts to generate a Glu aptamer did not result in any sequences that could be firmly identified as responding to Glu with sufficient confidence because we could not exclude coordination with sodium cations. Attempts to use Cu(II) lead only to Cu(II)-sensitive receptors (removal of Cu²⁺).

FIG. 26A shows the result of Cu(II)-Leu (direct selection)-aptamer 2. Cu(II)-Leu(direct)-2 is a minimized aptamer initially identified as a promising hit. (sequence 02 below). We focus on sequences that were above 1% of a pool (nine fulfilled this initial criteria, see below), ThT displacement midpoint<20 μM, and selectivity over lie and Phe (identified as important based on Leu2.0 crossreactivity). From those that bound, 01 was eliminated based on its preference for Ile, 03 because it did not respond to Leu, 04 because of cross-reactivity with Phe, with 02 selected as a candidate for testing on clinical samples. FIG. 26B shows the results of follow-up testing, ThT displacement. It was hypothesized that methionine is the potential interferent that led to failure of sensors in blind tests. This discovery needs to be taken into account when final sensors are chosen. FIG. 26C shows that same structure of Cu(II)-Leu(direct)-2 re-appears in other Cu(II) directed selections, with larger libraries, when counterselection with Ile is introduced. A selection using N₄₄ randomized regions (capital font in the aptamer on the right), in which we attempted to introduce additional structure switching mechanisms.

FIG. 27A shows a specific family of voriconazole-binding three-way junctions, despite being common, are eliminated from direct selections by exceptionally poor interactions with capture oligonucleotides, which was prevented in Vor1.0 by structure switching. FIG. 27B shows other voriconazole data.

### DETAILED DESCRIPTION OF THE DISCLOSURE

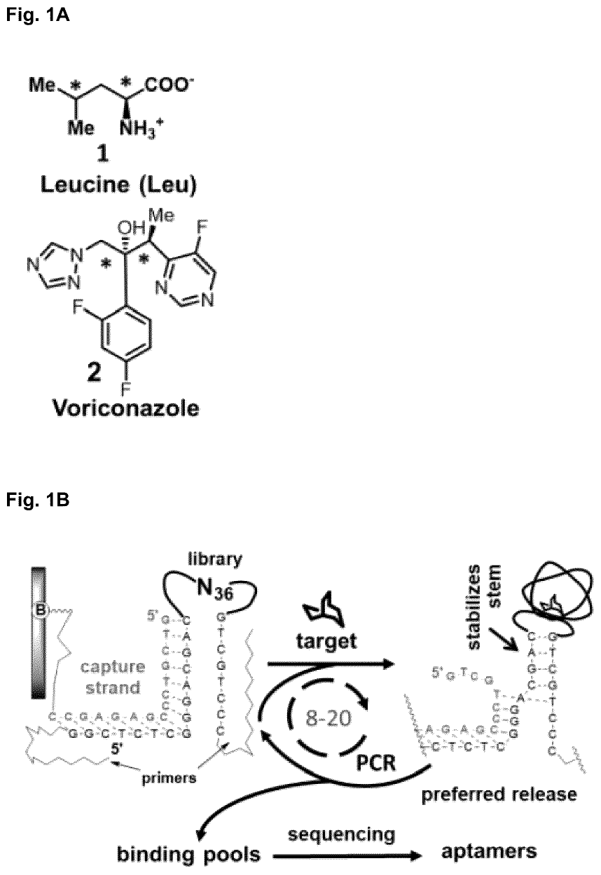

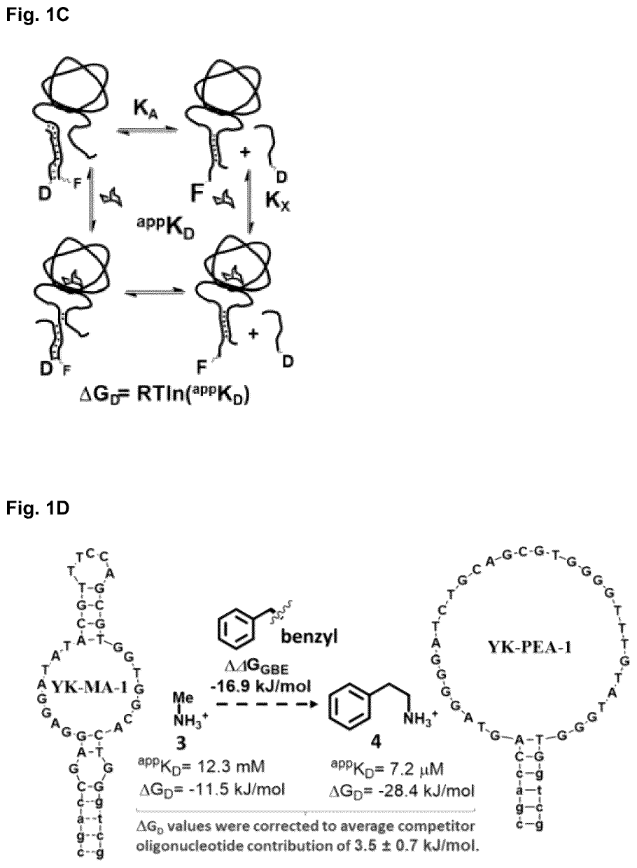

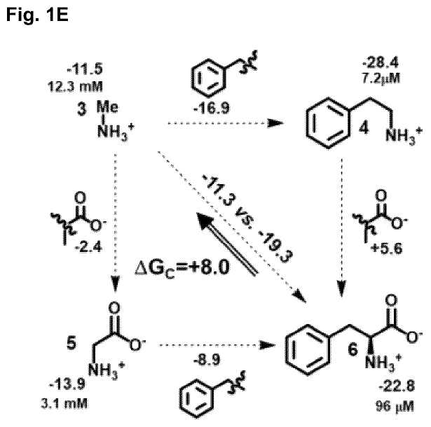

In previous studies, it was repeatedly unable to isolate DNA aptamers for two clinically important molecules, the amino acid leucine (Leu, 1) and the antifungal agent voriconazole (2) (FIG. 1A). Aptamers to detect blood leucine levels could be used to rapidly clarify false positives during newborn screening for maple syrup urine disease (MSUD) (11, 6). Further, we sought to expand on our success with vancomycin sensing (12), and isolate receptors that could be used for voriconazole therapeutic monitoring (13). Our attempts were variations of selections based on target-induced stem closure (FIG. 1B) (14, 15). In this approach, oligonucleotide libraries with internal random 36-mer regions are immobilized via 5′-primer regions that hybridize with tethered capture sequences. Potential aptamers hybridized on columns are released by interactions with unmodified targets in solution, which can stabilize stem formation upon displacement (FIG. 1B).

Prior failure to isolate DNA aptamers for leucine was surprising because RNA aptamers had been previously isolated via leucine-tethered affinity columns (16). Similarly, voriconazole should have been a straightforward target due to its aromatic surfaces and heteroatoms. However, one could neither adapt reported aptamers cross-reactive with the azole class of antifungals (17) as sensor components (8, 12), nor one could isolate new specific aptamers. These two seemingly unrelated targets, with significantly different molecular weights, share proximate pairs of sterically crowded carbons (FIG. 1A), which inspired us to pursue a broader understanding of the general relationships between target structures and outcomes of highly standardized selections. The aim was a generalizable approach to aptamer isolation when other, standard methods fail.

Accordingly, one embodiment of the present disclosure is a method for preparing an aptamer for a target molecule. This method comprises the steps of: (a) screening and selecting a candidate aptamer for the target molecule through standard selection protocols; (b) modifying the candidate aptamer by having an N₂₂ random insertion to form a library used to identify a sequence that recognizes at least one functional group on the target molecule; (c) generating a library of random 22-mers with the sequence identified in step (b) positioned next to the closing stem of the candidate aptamer; (d) performing a functional-group binding free energy analysis for aptamers from the library generated in step (c); and (e) identifying the aptamer that has the highest affinity for the target molecule.

As used herein, “standard selection protocols” for aptamers include, e.g., those that are based on affinity separation of binding and non-binding oligonucleotides. In the context of small molecules, the protocols often include, e.g., chemically modifying the target small molecules and attaching them to a solid-state matrix.

Non-limiting exemplary target molecules include: ammonia, glycinamide, methylamine, phenylethylamine, glycine, phenylalanine, methylbutylamine (1-Amino-3-methylbutane hydrochloride), tryptamine, methylene blue, histamine, serotonin, tryptophan, melatonin, L-DOPA (3,4-Dihydroxy-L-phenylalanine), norepinephrine, epinephrine, GABA (γ-Aminobutyric acid), GABA-amid (4-Aminobutyramide hydrochloride), glutamine, phenylalaninamide, tyrosinamide, leucine, tyrosine, tyramine, agmatine, arginine, dopamine, voriconazole, Cp*Rh(III) (pentamethylcyclo-pentadienylrhodium(III) chloride), Leu-Cp*Rh(iii), Leu-Cu(ii). In some embodiments, for example, the target molecule is leucine or voriconazole.

As used herein, an “aptamer” is a short, single-stranded sequence of artificial DNA, RNA, XNA, or peptide that bind a specific target molecule, or family of target molecules. Aptamers can replace antibodies in many biotechnology applications such as immunoassays including, e.g., enzyme-linked immunosorbent assay (ELISA), western blot, immunohistochemistry (IHC), and flow cytometry. Aptamers can also be used as therapeutics, functioning as agonists or antagonists of their ligand. The ability of aptamers to reversibly bind molecules such as proteins makes them useful in drug delivery systems and for controlled release of therapeutic biomolecules.

Another embodiment of the present disclosure is an aptamer disclosed herein, including those identified by the method disclosed herein. In some embodiments, the aptamer has one of the following structures:

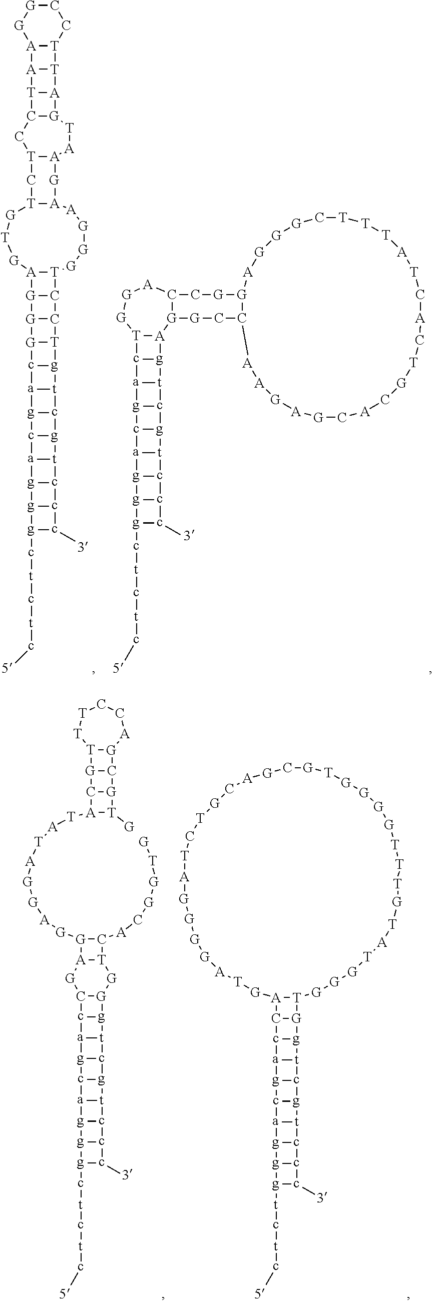

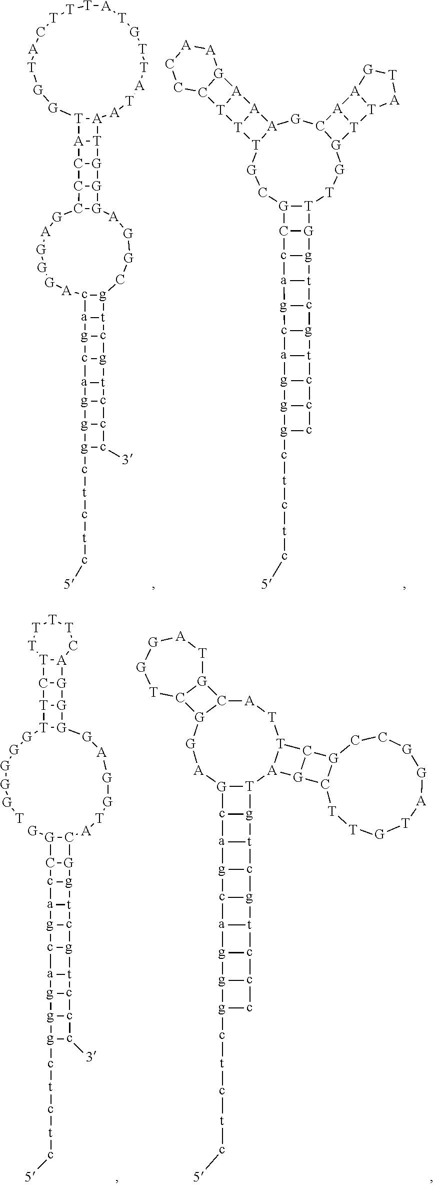

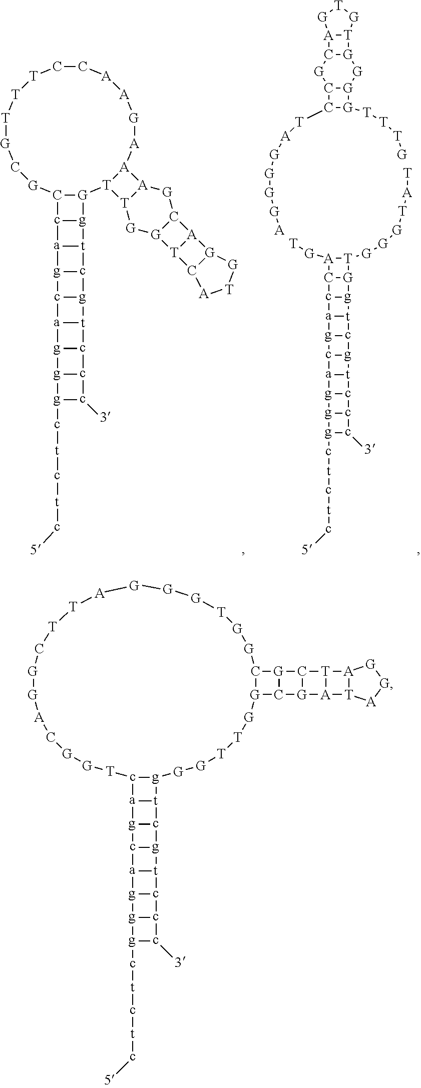

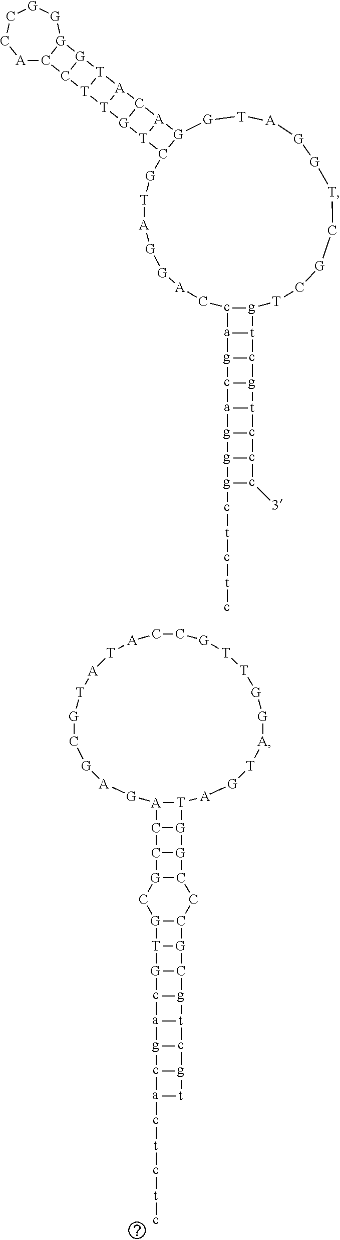

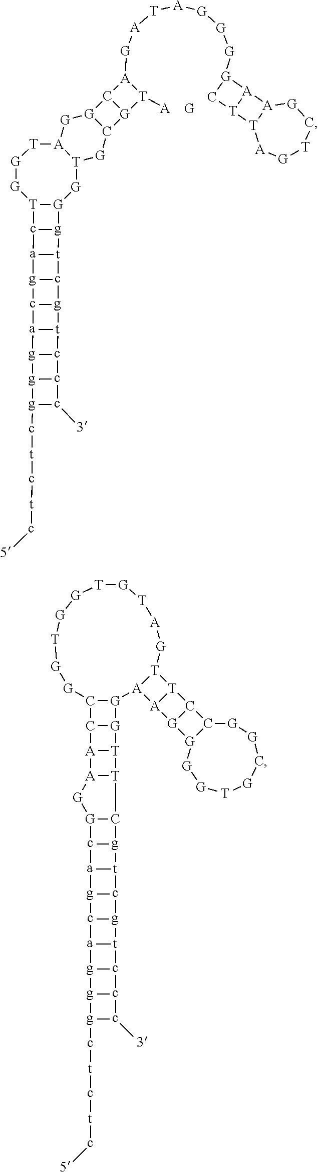

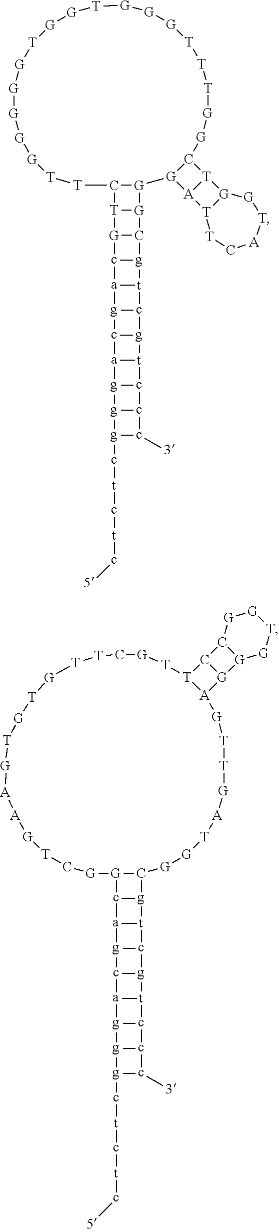

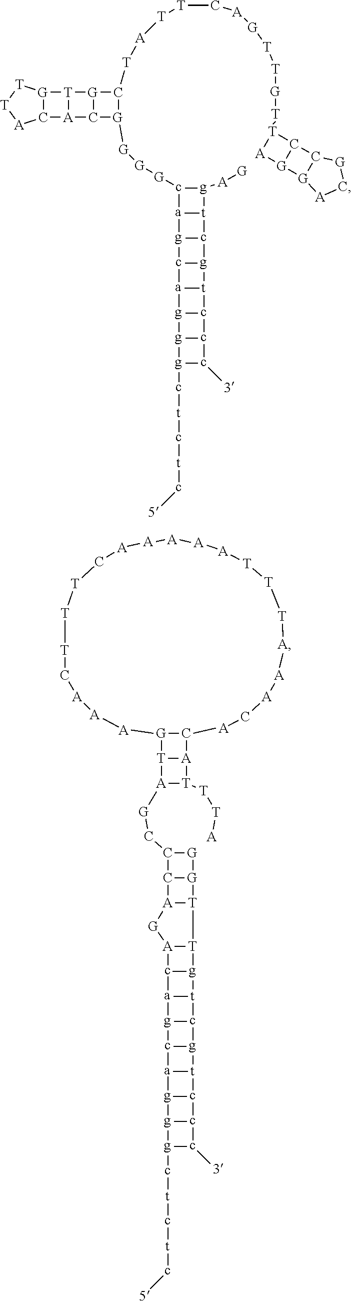

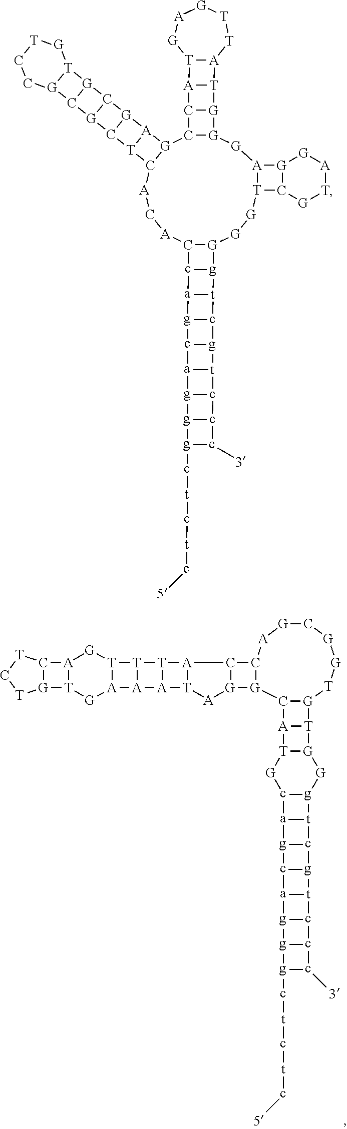

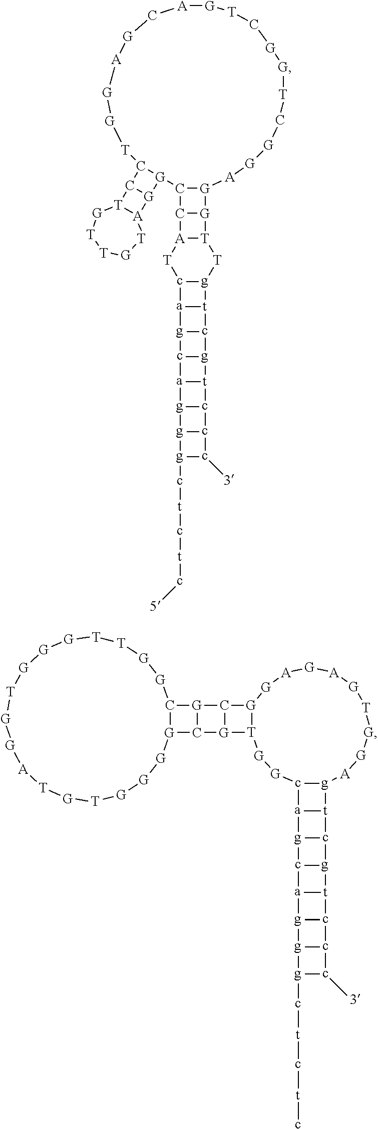

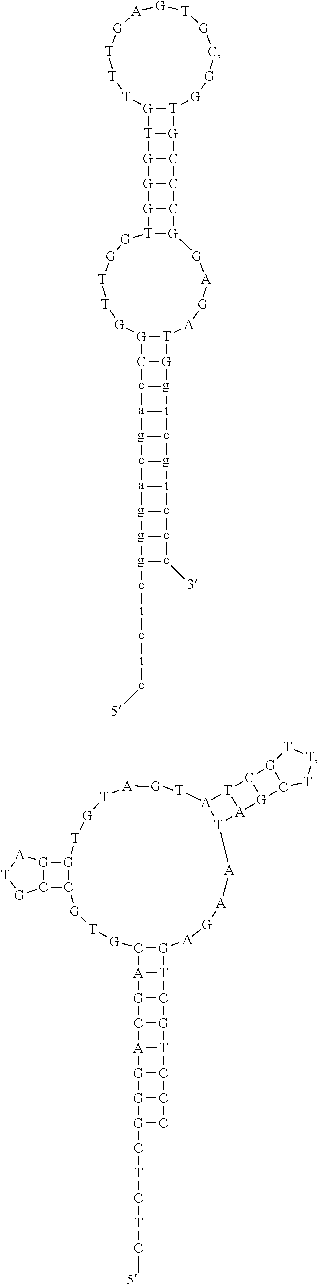

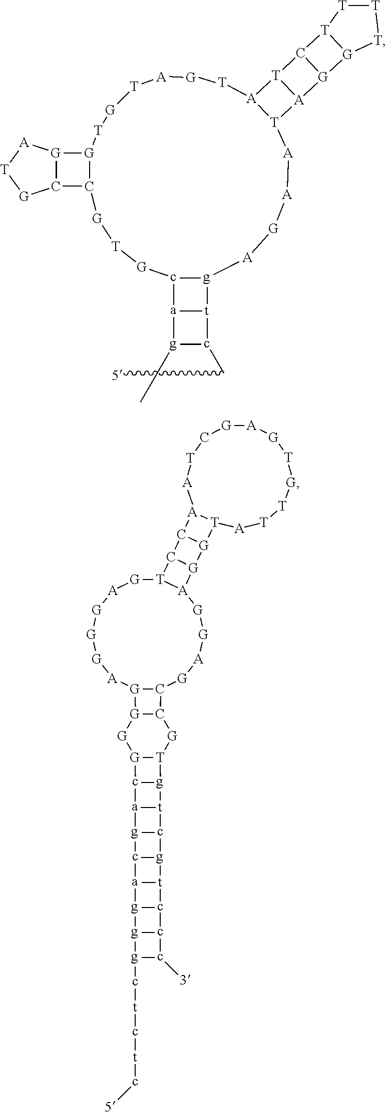

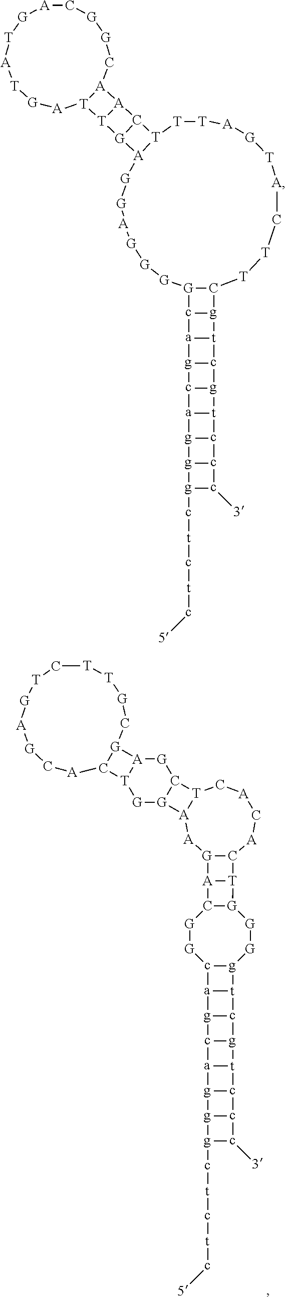

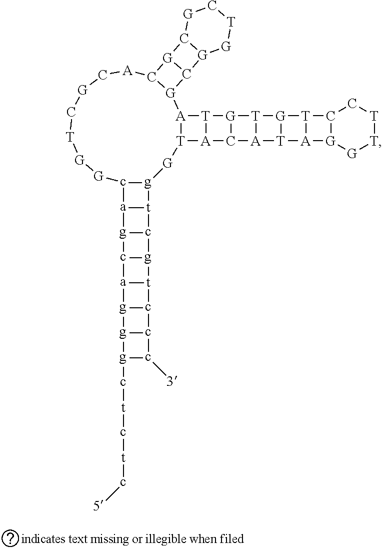

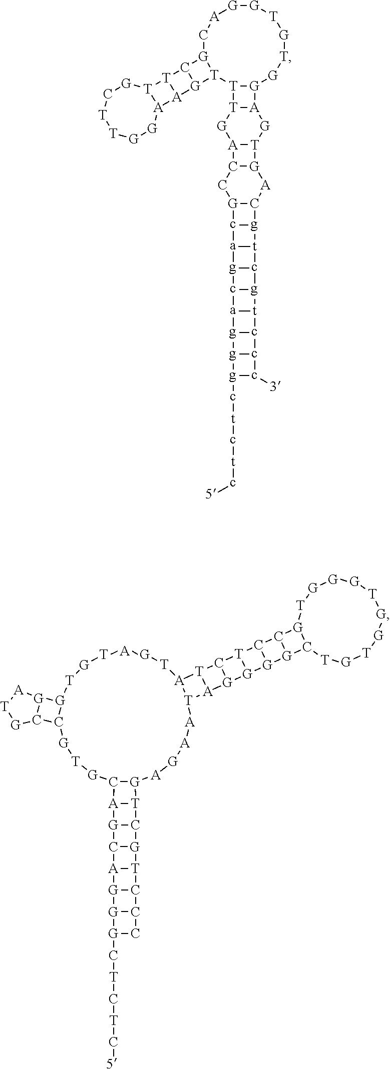

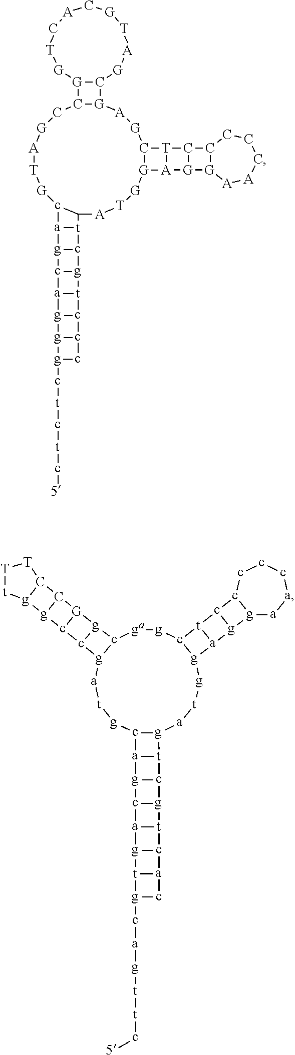

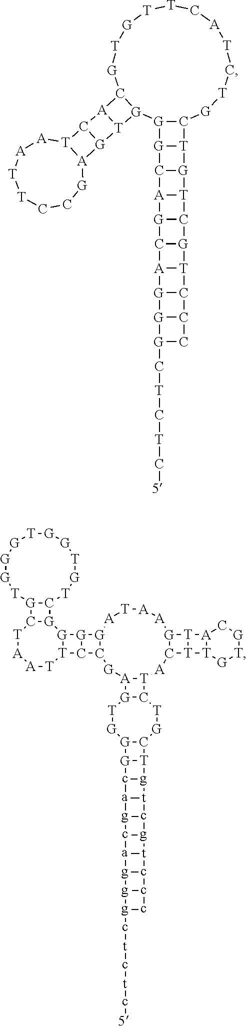

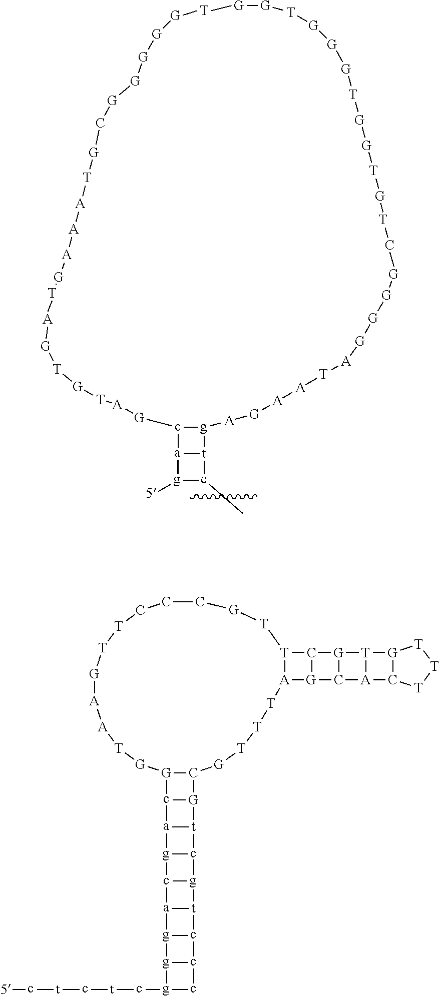

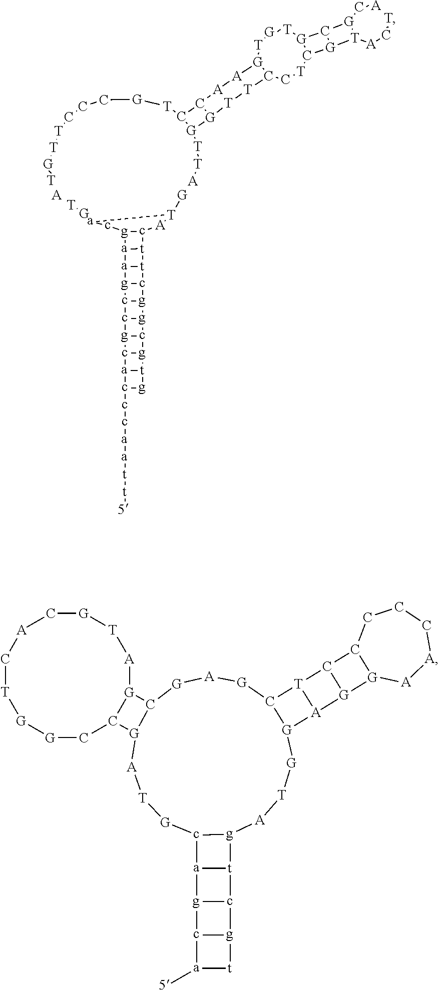

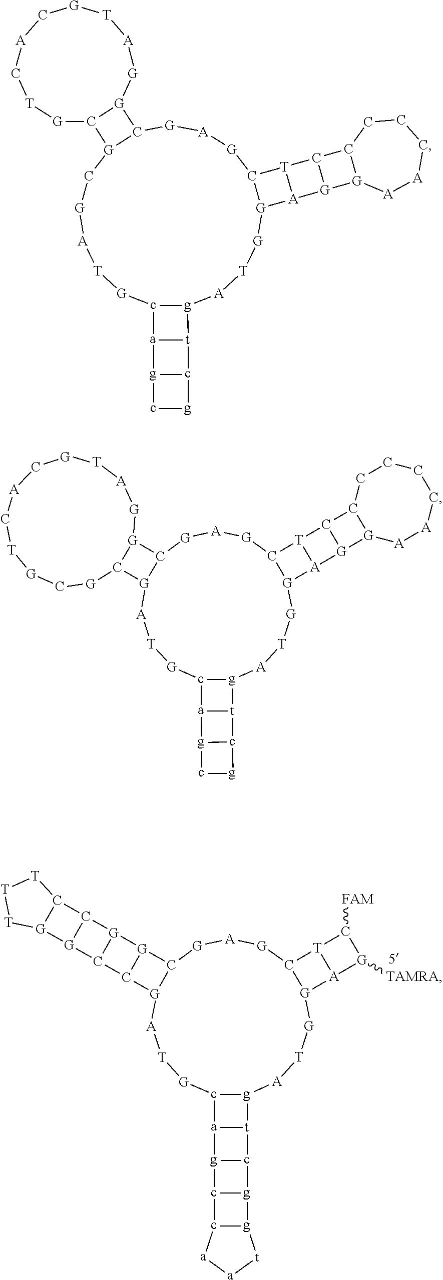

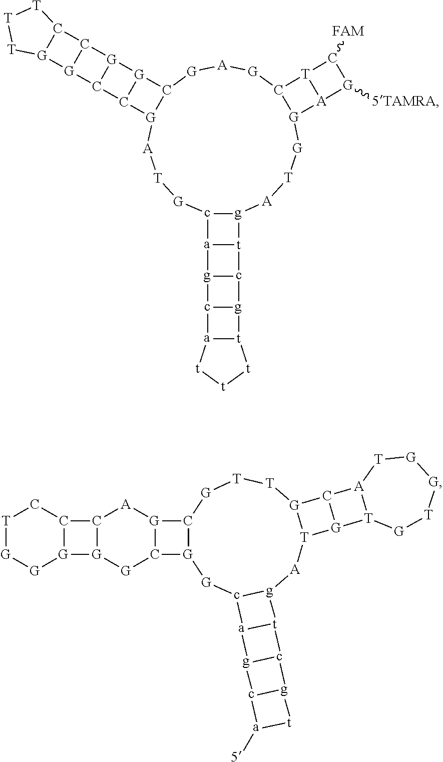

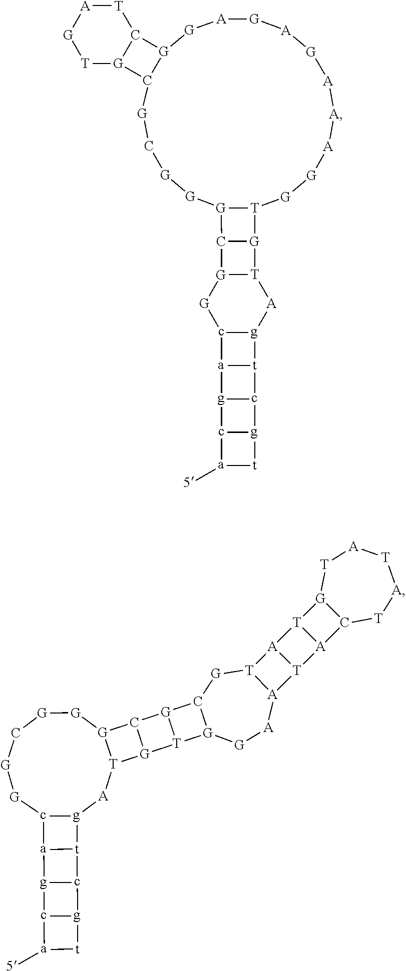

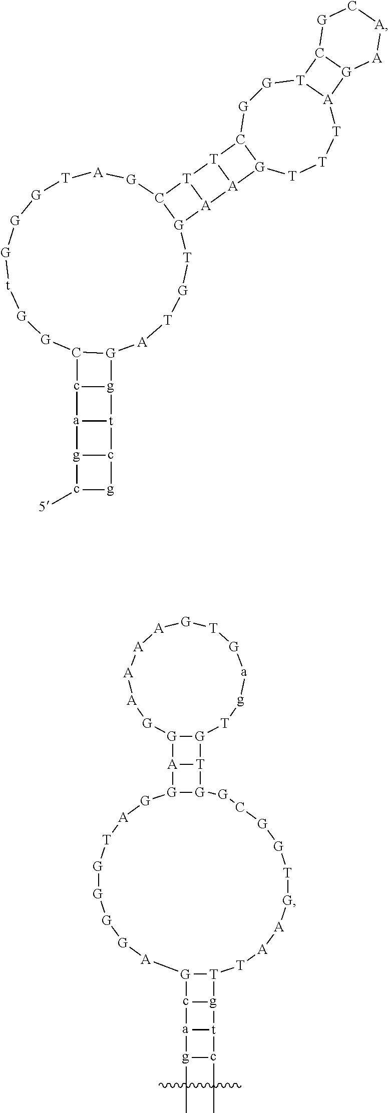

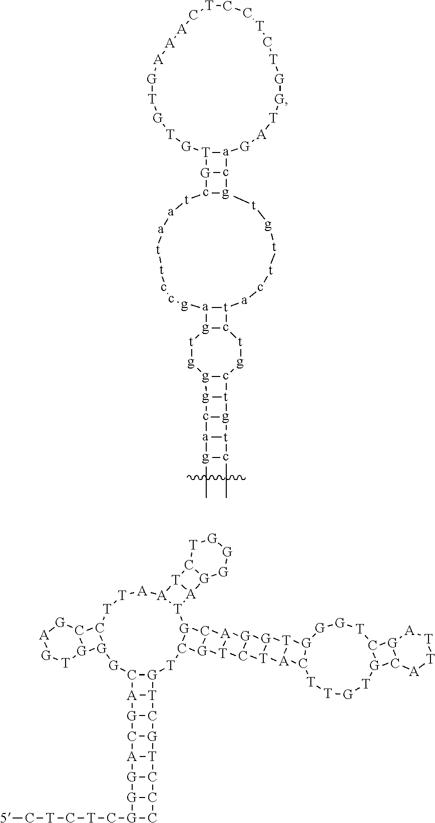

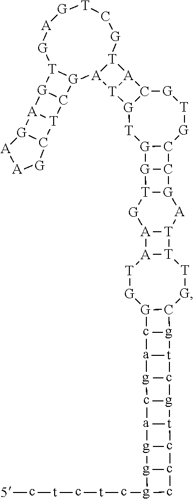

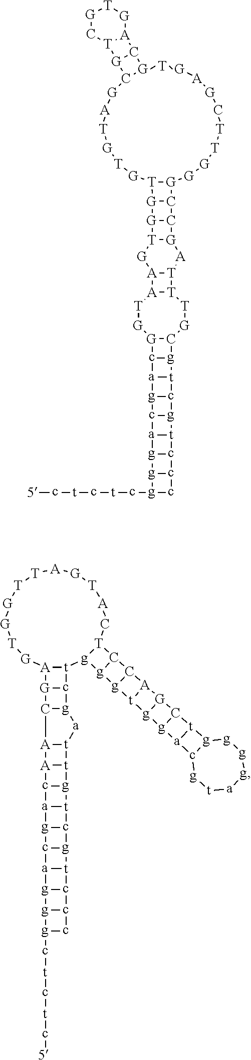

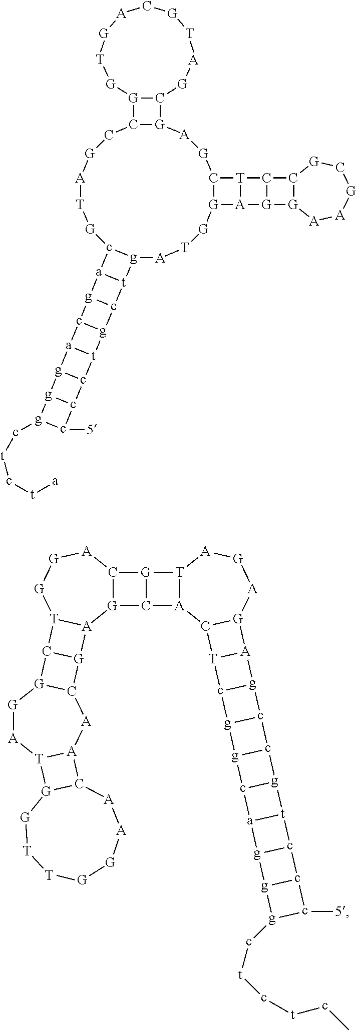

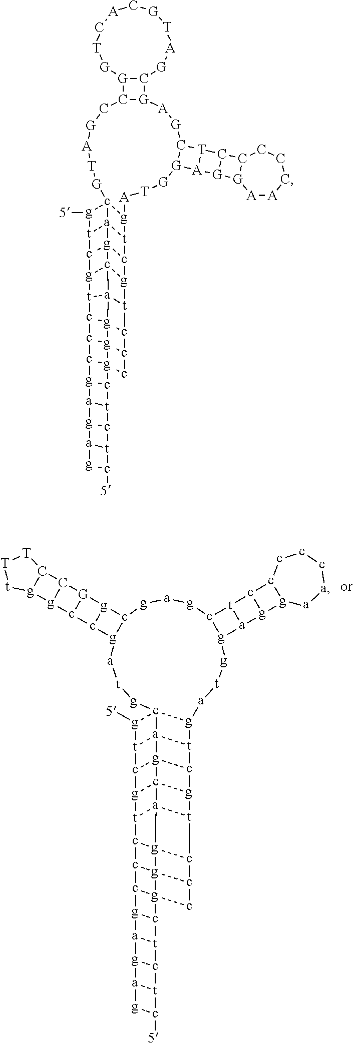

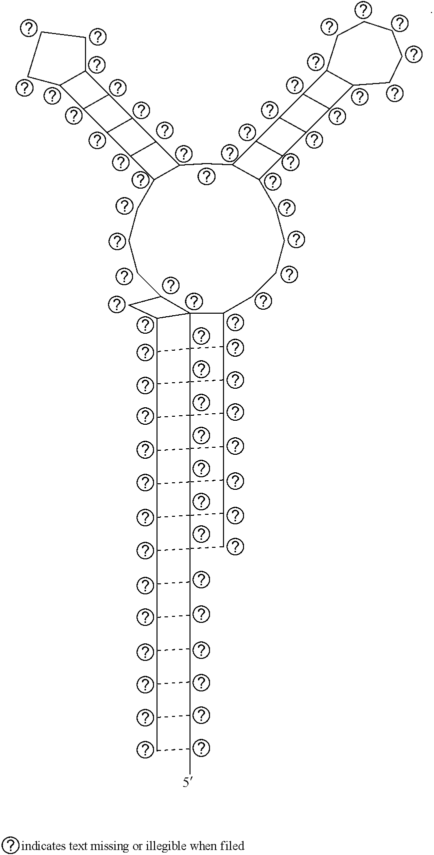

Still another embodiment of the present disclosure is a composition, including pharmaceutical compositions, comprising one or more aptamer disclosed herein, including those identified and prepared by the method disclosed herein.

A further embodiment of the present disclosure is a method for treating or ameliorating the effects of a condition in a subject in need thereof. This method comprises administering to the subject an effective amount of one or more aptamer disclosed herein, including those identified and prepared by the method disclosed herein or the composition disclosed herein.

Yet another embodiment of the present disclosure is a kit for treating or ameliorating the effects of a condition in a subject in need thereof, comprising an effective amount of one or more aptamer disclosed herein, including those identified and prepared by the method disclosed herein or the composition disclosed herein, packaged with its instructions for use.

The kits may also include suitable storage containers, e.g., ampules, vials, tubes, etc., for each compound of the present disclosure (which, e.g., may be in the form of pharmaceutical compositions) and other reagents, e.g., buffers, balanced salt solutions, etc., for use in administering the active agents to subjects. The aptamers and/or pharmaceutical compositions of the disclosure and other reagents may be present in the kits in any convenient form, such as, e.g., in a solution or in a powder form. The kits may further include a packaging container, optionally having one or more partitions for housing the aptamers and/or pharmaceutical compositions and other optional reagents.

As used herein, a “condition” refers to a disease or disorder that is a current or potential target of an aptamer as therapeutic. Non-limiting examples of such condition include Age-related macular degeneration, myasthenia gravis, Acute myeloid leukaemia, Percutaneous coronary intervention, Thrombotic microangiopathies and carotid artery disease, Cardiopulmonary bypass to maintain steady state of anticoagulation, Type 2 diabetes, diabetic nephropathy, Multiple myeloma and non-Hodgkin's lymphoma, chronic inflammatory disease, progressive malignant prostate disease, viral infection, lupus, migraine, epithelial hyperproliferative disease, septic shock, Acute respiratory distress syndrome (ARDS), and combinations thereof.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition or halt the progression of disease development. However, because every treated subject may not respond to a particular treatment protocol, regimen, process, or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

In the present disclosure, an “effective amount” or “therapeutically effective amount” of an aptamer or pharmaceutical composition is an amount of such an aptamer or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of an aptamer or pharmaceutical composition according to the disclosure will be that amount of the compound or composition, which is the lowest dose effective to produce the desired effect with no or minimal side effects. The effective dose of an aptamer or pharmaceutical composition according to the present disclosure may be administered as two, three, four, five, six or more sub-doses, administered separately at appropriate intervals throughout the day.

An aptamer, composition, or pharmaceutical composition of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, an aptamer, composition, or pharmaceutical composition of the present disclosure may be administered in conjunction with other treatments. An aptamer, composition, or pharmaceutical composition of the present disclosure may be encapsulated or otherwise protected against gastric or other secretions, if desired.

The compositions or pharmaceutical compositions of the present disclosure are pharmaceutically acceptable and comprise one or more active ingredients in admixture with one or more pharmaceutically-acceptable carriers or diluents and, optionally, one or more other compounds, drugs, ingredients and/or materials. Regardless of the route of administration selected, the compounds/compositions/pharmaceutical compositions of the present disclosure are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, PA.). More generally, “pharmaceutically acceptable” means that which is useful in preparing a composition that is generally safe, non-toxic, and neither biologically nor otherwise undesirable and includes that which is acceptable for veterinary use as well as human pharmaceutical use.

Pharmaceutically acceptable carriers and diluents are well known in the art (see, e.g., Remington, The Science and Practice of Pharmacy (21^{st} Edition, Lippincott Williams and Wilkins, Philadelphia, PA.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier or diluent used in a composition of the disclosure must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

The compositions or pharmaceutical compositions of the present disclosure may, optionally, contain additional ingredients and/or materials commonly used in such compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The formulations may be presented in unit-dose or multi-dose sealed containers, for example, ampules and vials, and may be stored in a lyophilized condition requiring only the addition of the sterile liquid carrier or diluent, for example water for injection, immediately prior to use. Extemporaneous injection solutions and suspensions may be prepared from sterile powders, granules and tablets of the type described above.

The following examples are provided to further illustrate the methods of the present disclosure. These examples are illustrative only and are not intended to limit the scope of the disclosure in any way.

### EXAMPLES

### Example 1

### Methods

### General

Chemicals were purchased from Sigma-Aldrich Co. (St. Louis, MO) unless otherwise noted. Oligonucleotides were ordered from Integrated DNA Technologies (Coralville, IA or Morrisville, NC) and used as received.

### Selection Procedures

Our general selection procedures have been reported previously (41). All selections were performed either directly or closely supervised by a single person (KY) to maximize the comparability of aptamers through consistent decision-making regarding the choices of target concentration ranges, numbers of pre-washes, and inclusion of counter-targets and their concentrations (if any). An example of a selection flow chart illustrating decision-making at each step is shown in Nakatsuko et al., 2018 (5).

Standard desalted oligonucleotides were used for the library and primers as described in Table 1. Purification by HPLC was used for fluorophore-conjugated oligonucleotides. All oligonucleotides were dissolved in nuclease-free water and stored at −20° C. Each PCR was run with an initial cycle at 95° C. for 2 min, followed by N cycles of [95° C. for 15 s→60° C. for 20 s→72° C. for 30 s], ending with a single cycle at 72° C. for 2 min. The PCR runs were 11±2 cycles. Phosphate-buffered saline (PBS), pH 7.4 (Corning, Corning, NY) with 2 mM MgCl₂ and sometimes 5 mM KCl were used for selections as indicate in Table 2.

TABLE 1
Oligonucleotide sequences used in selections.
The oligonucleotide set for the N30, N36 or N45
random libraries (5′ --> 3′), Ver.01

| | |
|---|---|
| Library | GGA GGC TCT CGG GAC GAC (N30, N36 or N45 )GTC |
| | GTC CCG CCT TTA GGA TTT ACA G (SEQ ID NO: 1) |
| Capture | GTC GTC CCG AGA GCC ATA /3BioTEG/ (SEQ ID NO: 2) |
| strand | |
| primer | GGA GGC TCT CGG GAC GAC (SEQ ID NO: 3) |
| (Forward) | |
| primer | /5Biosg/ CTG TAA ATC CTA AAG GCG GGA CGA C (SEQ |
| (Reverse) | ID NO: 4) (if apply cloning for sequencing, |
| | non-biotin sequence is needed) |
| NGS primer, | GAA CAA CCG AAA AAA GGG GAA CCC AAG ACC CAA AGG |
| Forward | AGG CTC TCG GGA CGA C (SEQ ID NO: 5) |
| NGS primer, | GGA AAA ACC CAA GGA CCA AAC TGT AAA CCT GTA AAT |
| Reverse | CCT AAA GGC GGG ACG AC (SEQ ID NO: 6) |

The oligonucleotide set for the N36 random library, Ver.02

| | |
|---|---|
| Library | CAA TAT GAC CTC ACT CTC GGG ACG AC(N36)G TCG TCC |
| | CTA CTC AGG TCA CGC AGC (SEQ ID NO: 7) |
| Capture | GTC GTC CCG AGA GTG TAA /3BioTEG/ (SEQ ID NO: 8) |
| strand | |
| primer | CAA TAT GAC CTC ACT CTC GGG ACG AC (SEQ ID NO: |
| (Forward) | 9) |
| primer | /5Biosg/GCT GCG TGA CCT GAG TAG GGA CGA C (SEQ |
| (Reverse) | ID NO: 10) |
| NGS primer, | GAA CAA CCG AAA AAA GGG GAA CCC AAG ACC CAA ACA |
| Forward | ATA TGA CCT CAC TCT CGG (SEQ ID NO: 11) |
| NGS primer, | GGA AAA ACC CAA GGA CCA AAC TGT AAA CCT GTA AAT |
| Reverse | CCT GCT GCG TGA CCT GAG TAG (SEQ ID NO: 12) |

Cp*Rh (iii) insertion library set

| | |
|---|---|
| Library | CTC TCG GGA CGA CGG GTG AGC CTT AAT C(N22)A CGT |
| | GTT CAT CTG CTG TCG TCC C (SEQ ID NO: 13) |
| Capture | GTC GTC CCG AGA GCC ATA /3BioTEG/ (SEQ ID NO: |
| strand | 14) |
| primer | CTC TCG GGA CGA CGG GTG AGC CTT AAT C (SEQ ID |
| (Forward) | NO: 15) |
| primer | /5Biosg/GGG ACG ACA GCA GAT GAA CAC GT (SEQ ID |
| (Reverse) | NO: 16) |
| NGS primer, | GAA CAA CCG AAA AAA GGG GAA CCC AAG ACC CAA AGG |
| Forward | AGG CTC TCG GGA CGA CGG GTG (SEQ ID NO: 17) |
| NGS primer, | AGG ACC AAA CTG TAA ACC TGT AAA TCC TAA AGT AAA |
| Reverse | GGC GGG ACG ACA GCA GAT GAA (SEQ ID NO: 18) |

Leucine insertion library set

| | |
|---|---|
| Library | GGA GGC TCT CGG GAC GAC(N22)GT GGG TGG TGT CGG |
| | GGA TAA GAG TCG TCC C (SEQ ID NO: 19) |
| Capture | GTC GTC CCG AGA GCC ATA/3BioTEG/ (SEQ ID NO: 20) |
| strand | |
| primer | GGA GGC TCT CGG GAC GAC (SEQ ID NO: 21) |
| (Forward) | |
| primer | /5Biosg/GGG ACG ACT CTT ATC CCC GAC ACC ACC CAC |
| (Reverse) | (SEQ ID NO: 22) |
| NGS primer, | GAA CAA CCG AAA AAA GGG GAA CCC AAG ACC CAA AGG |
| Forward | AGG CTC TCG GGA CGA C (SEQ ID NO: 23) |
| NGS primer, | GGA AAA ACC CAA GGA CCA AAC TGT AAA CCT GTA AAT |
| Reverse | CCT AAA GGC GGG ACG ACT CTT ATC CCC (SEQ ID NO: |
| | 24) |

Isoleucine insertion library set

| | |
|---|---|
| Library | GGA GGC TCT CGG GAC GAC(N22)TG GGG ATG CAG GTG |
| | GGT CGA TTG TCG TCC C (SEQ ID NO: 25) |
| Capture | GTC GTC CCG AGA GCC ATA/3BioTEG/ (SEQ ID NO: 26) |
| strand | |
| primer | GGA GGC TCT CGG GAC GAC (SEQ ID NO: 27) |
| (Forward) | |
| primer | /5Biosg/GGG ACG ACA ATC GAC CCA CCT GCA TCC CCA |
| (Reverse) | (SEQ ID NO: 28) |
| NGS primer, | GAA CAA CCG AAA AAA GGG GAA CCC AAG ACC CAA AGG |
| Forward | AGG CTC TCG GGA CGA C (SEQ ID NO: 29) |
| NGS primer, | GGA AAA ACC CAA GGA CCA AAC TGT AAA CCT GTA AAT |
| Reverse | CCT AAA GGC GGG ACG ACA ATC GAC CCA (SEQ ID NO: |
| | 30) |

TABLE 2
The oligonucleotide library and sequencing method
used for each aptamer isolation. The buffer for most targets
was PBS (+2 mM MgCl₂) except where indicated (**PBS +
2 mM MgCl₂ + 5 mM KCl).

| # | Aptamer ID | Target Name | Library | Sequencing |
|---|---|---|---|---|
| 1 | Leu 2.1-apt | Leucine | Leu insertion | NGS |
| | | | library | |
| 2 | Vor-apt | Voriconazole | N36. ver.02 | NGS |
| 3 | MEA-apt | Methylamine | N36. ver.01 | NGS |
| 4 | PHEA-apt | Phenylethylamine | N36. ver.01 | NGS |
| 5 | GLY-apt | Glycine | N36. ver.01 | Sanger |
| 6 | PHE-apt(1) | Phenylalanine | Ref. (7) | |
| 6 | PHE-apt(2) | | Ref. (7) | |
| 6 | PHE-apt(3) | | Ref. (7) | |
| 6 | PHE-apt(4) | | N36. ver.01 | NGS |
| 7 | MBA-apt | Methylbutylamine | N36. ver.01 | NGS |
| 8 | TRPA-apt | Tryptamine | N36. ver.01 | NGS |
| 9 | MB-apt | Methylene Blue | N36. ver.01 | Sanger |
| 10 | HIS-apt | Histamine | N36. ver.01 | Sanger |
| 11 | SRTN-apt | Serotonin | Ref. (5) | |
| 12 | TRP-apt | Tryptophan | N36. ver.01 | NGS |
| 13 | MLTN-apt | Melatonin | N36. ver.01 | Sanger |
| 14 | DOPA-apt | L-DOPA | N36. ver.01 | NGS |
| 15 | NE-apt | Norepinephrine | N36. ver.01 | Sanger |
| 16 | EPI-apt | Epinephrine | N36. ver.01 | Sanger |
| 17 | GABA-apt | GABA | N45 | NGS |
| 18 | GABM-apt | GABAmid | N36. ver.01 | NGS |
| 19 | GLN-apt | Glutamine | N36. ver.01 | Sanger |
| 20 | PAM-apt | Phenylalaninamide | N36. ver.01 | NGS |
| 21 | TYRA-apt | Tyrosinamide | N36. ver.01 | NGS |
| 22 | GAM-apt | Glycinamide | Leu insertion | NGS |
| | | | library | |
| 23 | TYR-apt | Tyrosine | N36. ver.01 | Sanger/NGS |
| 24 | TYM-apt | Tyramine | N36. ver.01 | NGS |
| 25 | AMT-apt | Agmatine | N36. ver.01 | NGS |
| 26 | ARG-apt | Arginine | N36. ver.01 | NGS |
| 27 | DA-apt | Dopamine | Ref. (5) | |
| 28 | Ammo-apt | Ammonia | N36. ver.01 | NGS |
| | Cp*Rh-apt | Cp*Rh(III)** | N30 | Sanger |
| | CpRhLeu-apt | Leu-Cp*Rh(III)** | Cp*Rh apt | NGS |
| | | | insertion | |
| | | | library | |
| | CuLeu-apt | Leu-Cu(II)** | Leu anchor | NGS |
| | | | insertion | |
| | | | library | |
| | CpRhIle-apt | Ile-Cp*Rh(III)** | Cp*Rh apt | NGS |
| | | | insertion | |
| | | | library | |
| | CuIle-apt | Ile-Cu(II)** | Ile anchor | NGS |
| | | | insertion | |
| | | | library | |

A general protocol where target concentrations are reduced in successive selection steps is as follows. Semiquantitative PCR was used to guide decision-making at each step (i.e., band densities in a gel were compared). When differences between the prewash in the previous selection step and the first wash in the next step did not change and bands remain visible, the target concentration was reduced by half and the selection was continued. If after three additional selection-amplification cycles, there were no increases in the band densities between the last prewash and the first wash for a target, the last cycle where differences were observed was sequenced. The assumption was that the step contained fractions responsive to the lowest target concentration. Selections (precise conditions) varied across targets but typically, selections were stopped at <20 cycles.

### Sequencing and Sequence Analysis

Due to the long-term nature of this project, selections for aptamers early in the study were carried out in combination with Sanger sequencing of the resulting selection pools. Aptamer pools selected later in the study were sequenced using next-generation sequencing (NGS). At the time of the transition, we ran both sequencing methods in parallel for one model target (tyrosine) and observed no differences in the top three aptamers identified. The sequencing methods for each target are noted in Table 2.

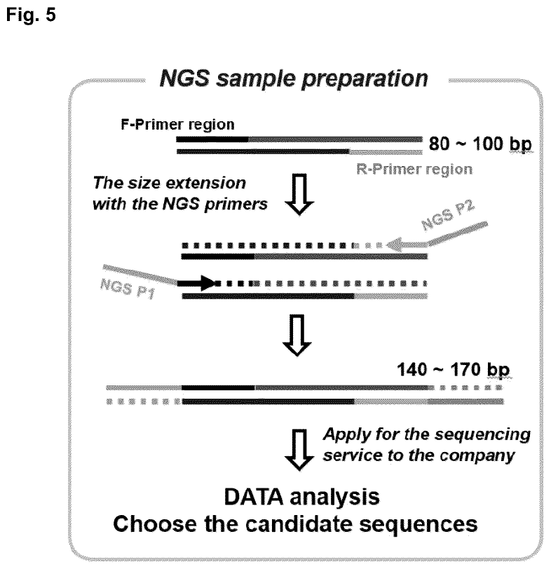

In the case of Sanger sequencing, the procedure was essentially the same as described in (41) where all sequences were screened for target binding using ThT displacement. For NGS sequencing, we used overlap extension PCR to increase sequence lengths to >140 bp for NGS (amplicon NGS service, Genewiz, South Plainfield, NJ). As shown below, we constructed the longer overhang NGS primers to include the 5′-ends of the partial sequences of each forward/reverse primer (F/R) (Table 1). The same PCR conditions were applied for longer primers. The extended NGS PCR products were purified using a PCR purification column (ThermoFisher Scientific, Waltham, MA). Sample concentrations were normalized following guidelines from the sequencing service. We ranked sequences by read numbers provided by Genewiz and analyzed convergent motifs. Analyses were carried out in Excel in combination with applicable online programs (e.g., AptaSUITE, https://drivenbyentropy.github.io/). NGS sample preparation is also shown in FIG. 5.

### Thioflavin T Dye Displacement to Screen for Aptamer-Target Interactions

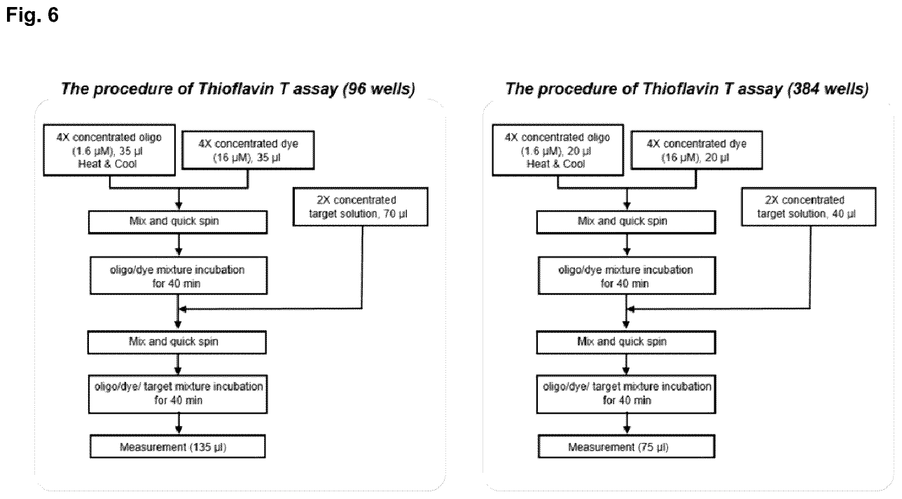

Based on the rankings by read number (high to low), typically, we would select at least five sequences from each selection pool and fold them computationally into 2D structures using mFold (http://www.unafold.org/mfold/applications/dna-folding-form.php) (42). From the predicted 2D structures, the candidate sequences would be truncated to reduce the terminal stems to 5-6 base pairs. Truncated sequences were used in the Thioflavin T (ThT) dye displacement assay. Oligonucleotide sequence lengths were typically 44±2 nucleotides for this step. Sequences underwent standard desalting purification. We used published ThT assay procedures (43) with buffer conditions as used in the selections. The ThT procedures are demonstrated in FIG. 6.

Oligonucleotides were first placed in boiling water for 5 min, removed, and allowed to cool to room temperature. The ThT dye solution was mixed with an equal volume of aptamer solution and incubated at room temperature for ˜40 min protected from light. During incubations, target solutions (2× aptamer concentrations) were prepared and then mixed with the ThT/oligo solutions and incubated for another >40 min. Aptamer final concentrations were 400 nM, while the ThT dye was 4 μM. Fluorescence measurements were carried out in triplicate in 96- or 384-well black plates using FlexStation II or SpectraMax5 microplate readers (Molecular Devices, San Jose, CA) to record ThT fluorescence spectra at excitation and emission wavelengths of 425 nm and 490 nm, respectively. The 50% signal maximum was used to compare relative aptamer-target affinities. Among the candidates, we advanced sequences with the highest relative target binding. If there were sequences that had high count in sequencing, yet little dye displacement, we would order such candidates in the FAM/Dab format.

### Displacement Assay

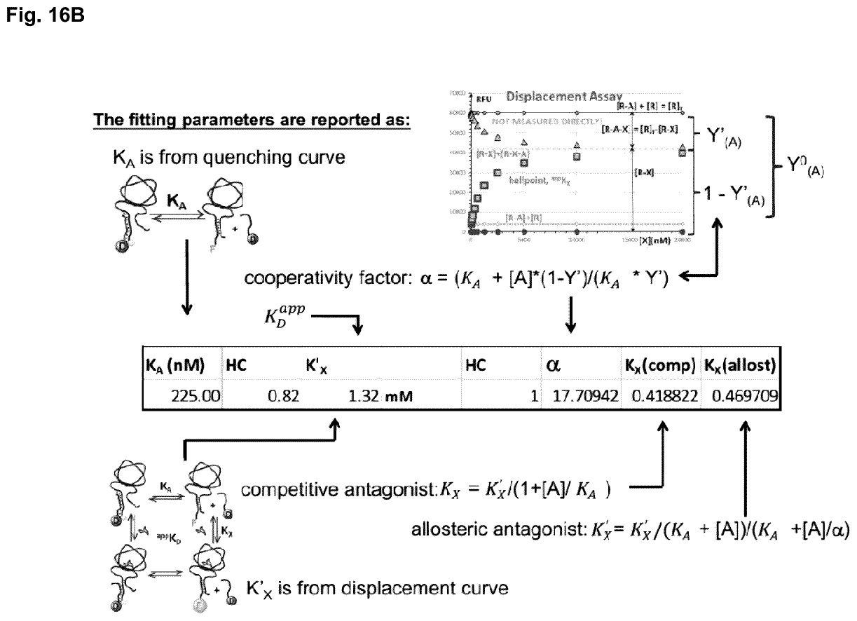

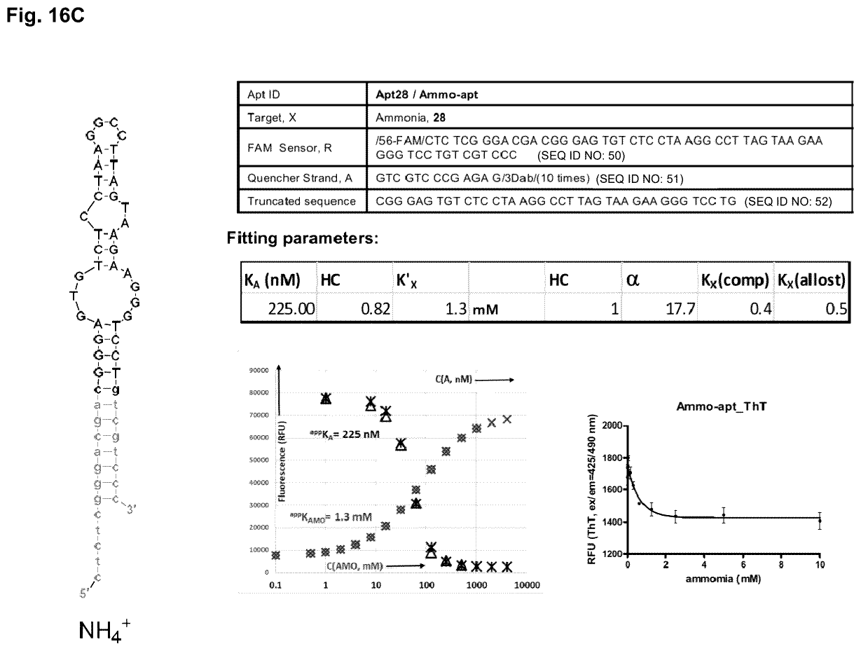

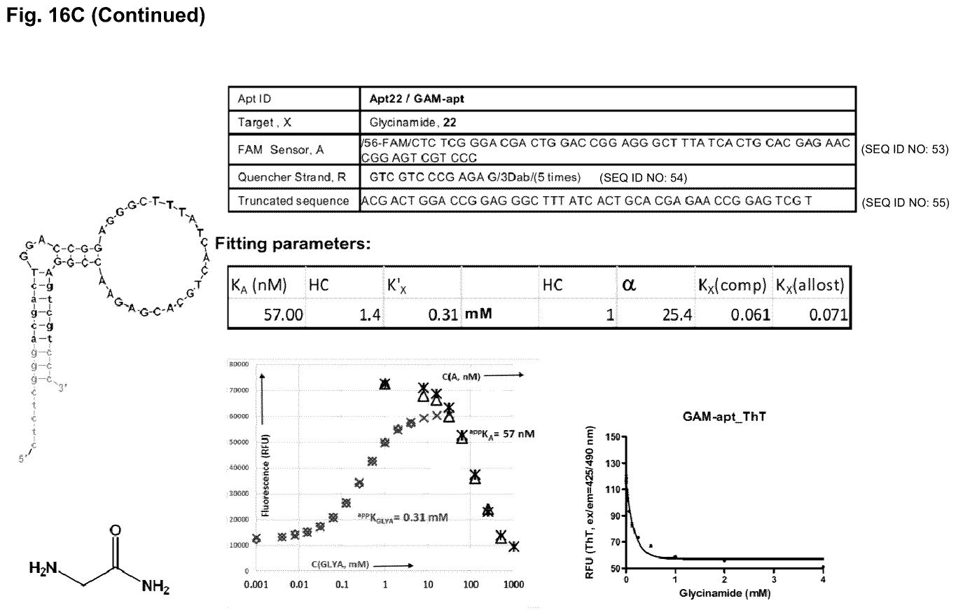

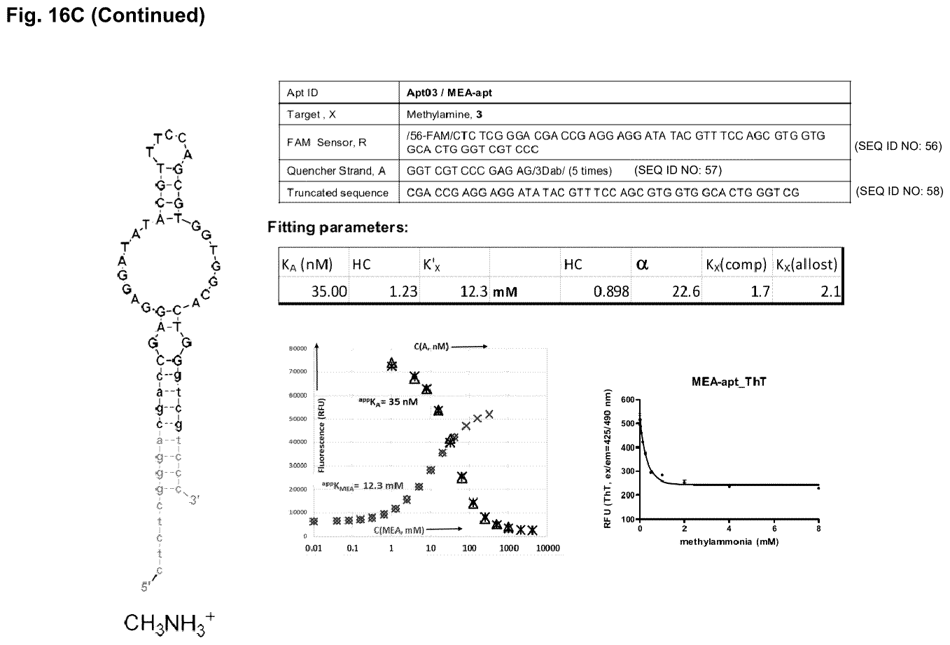

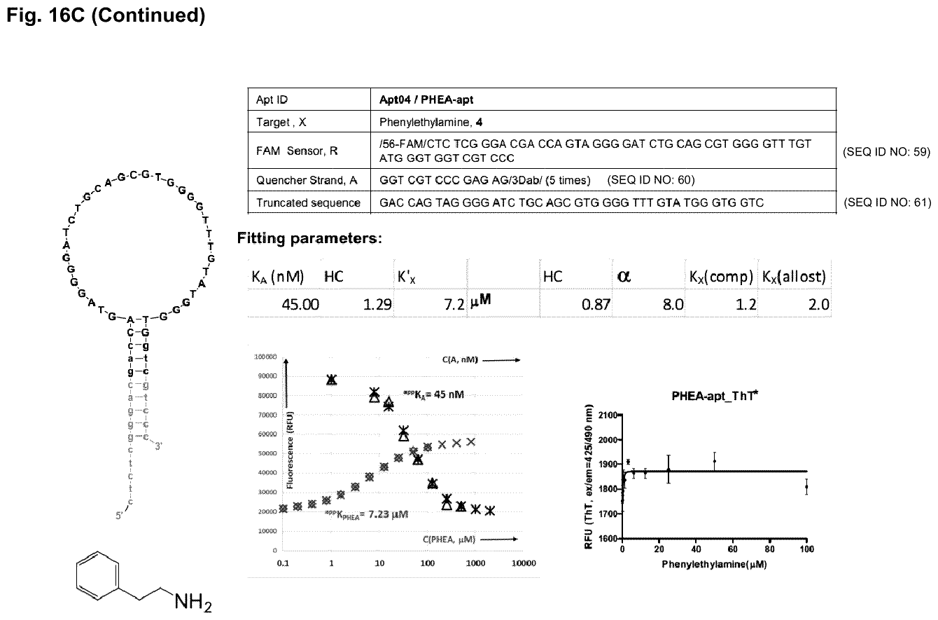

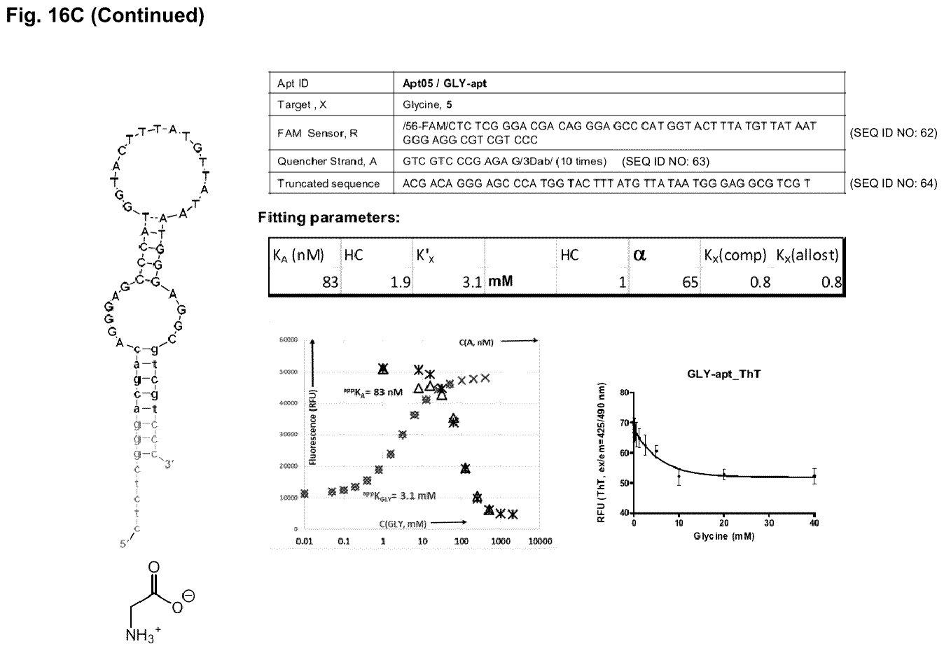

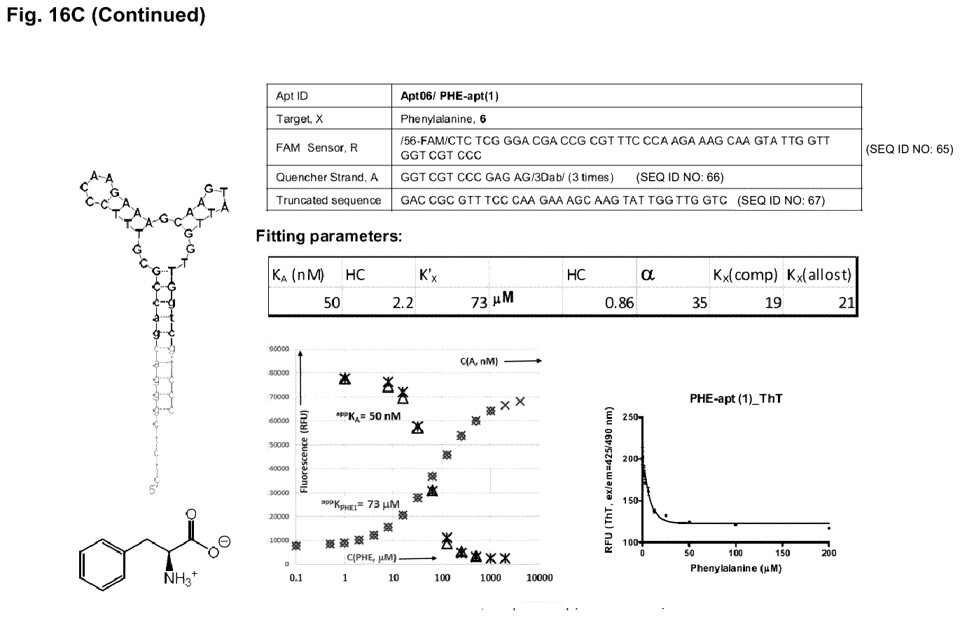

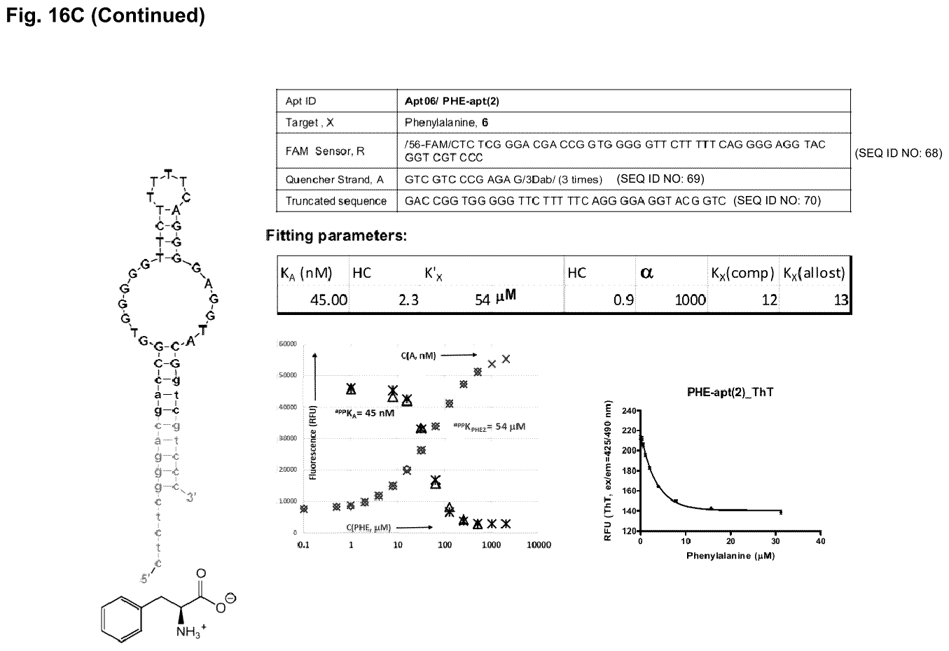

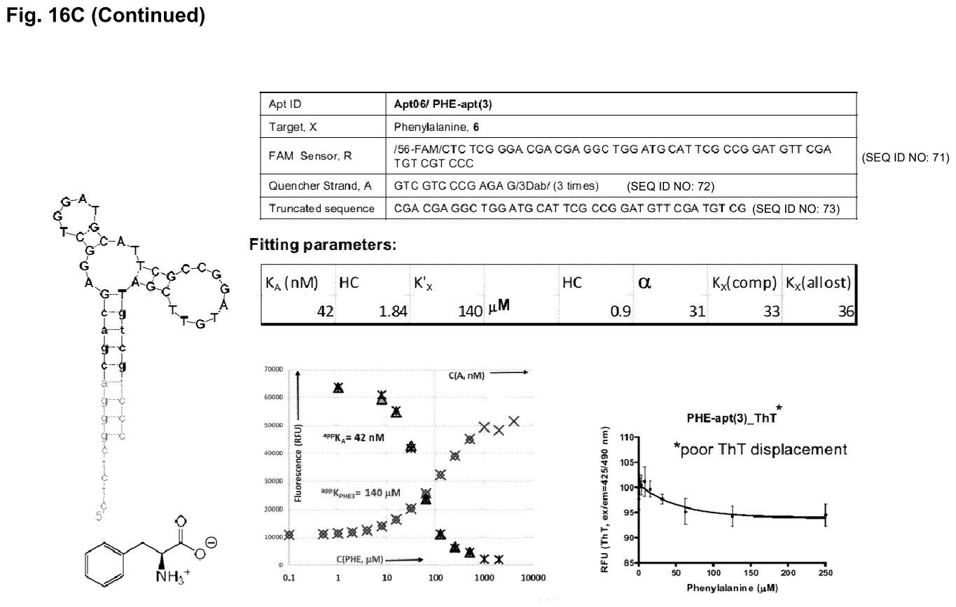

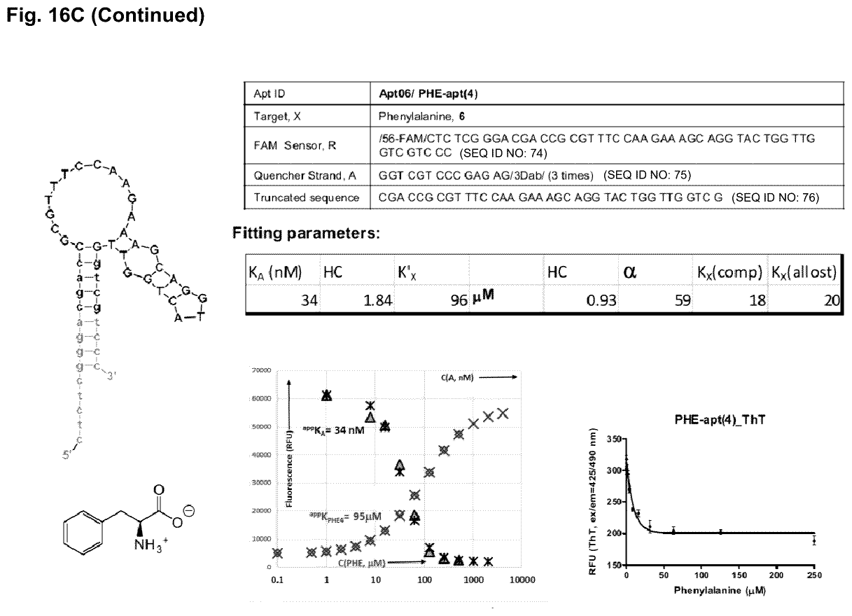

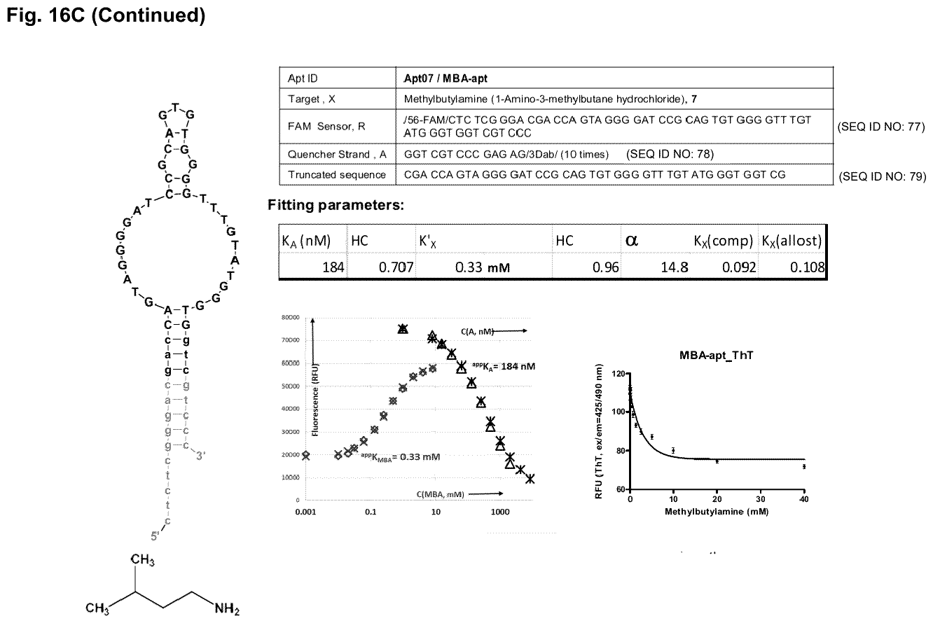

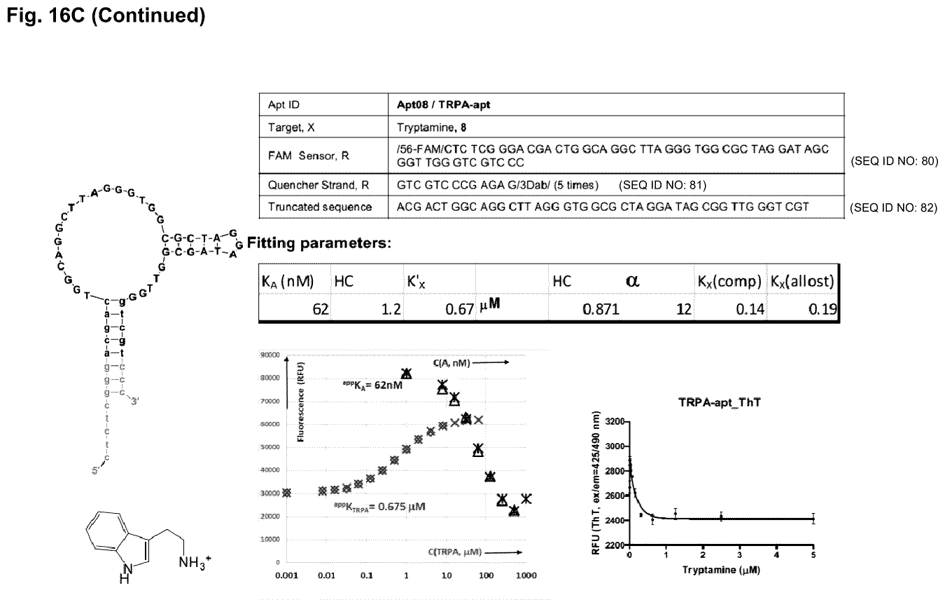

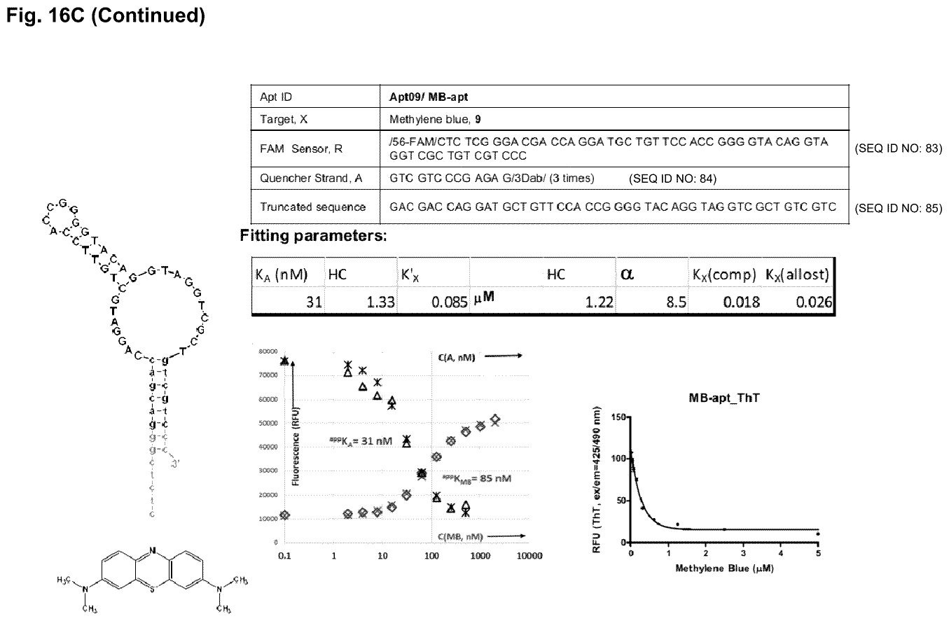

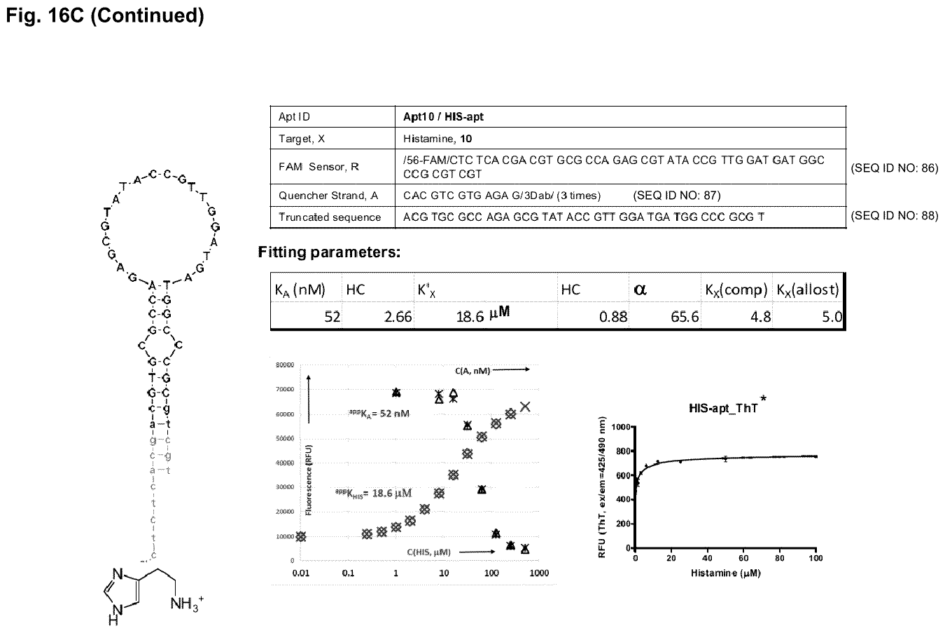

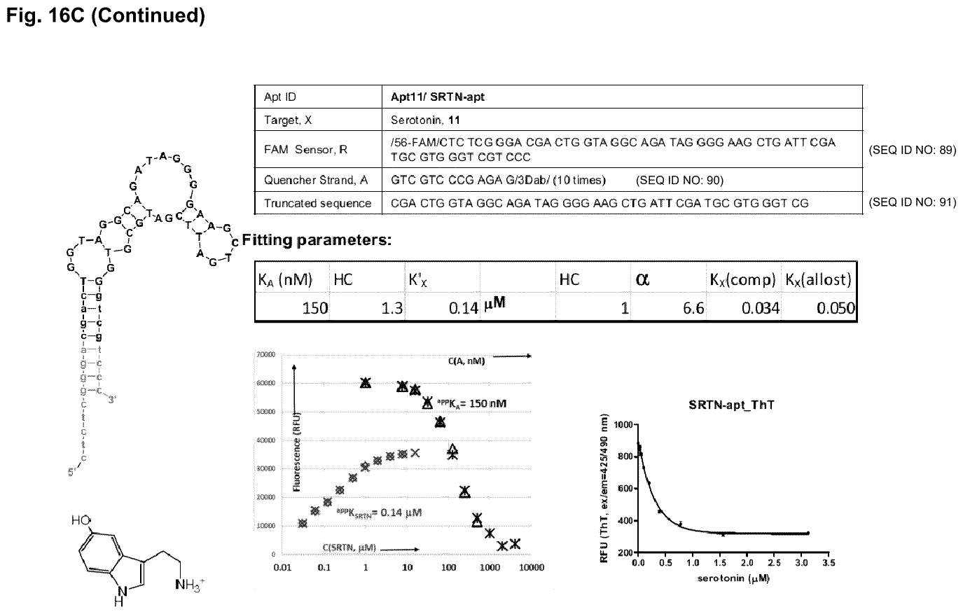

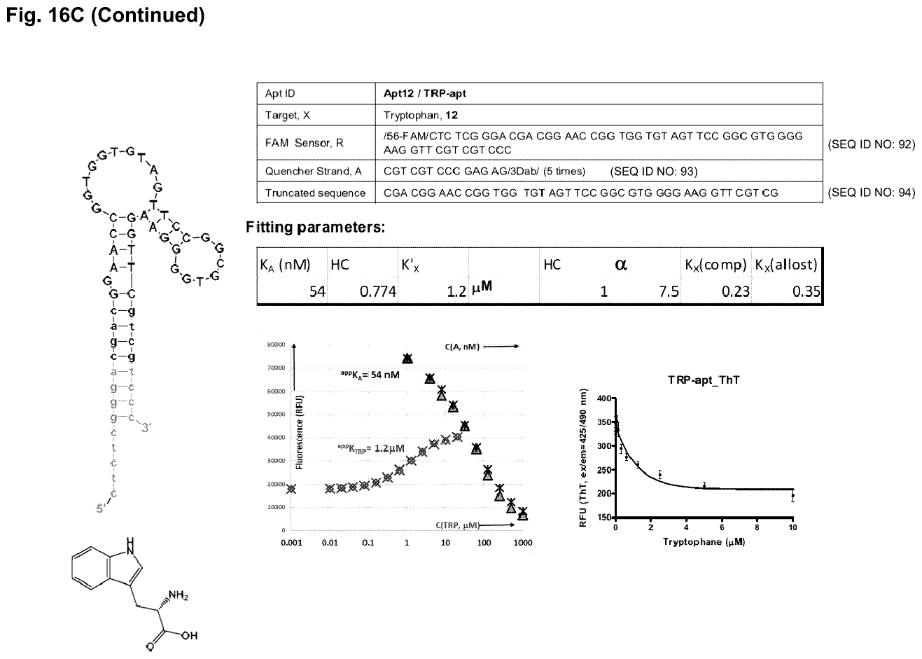

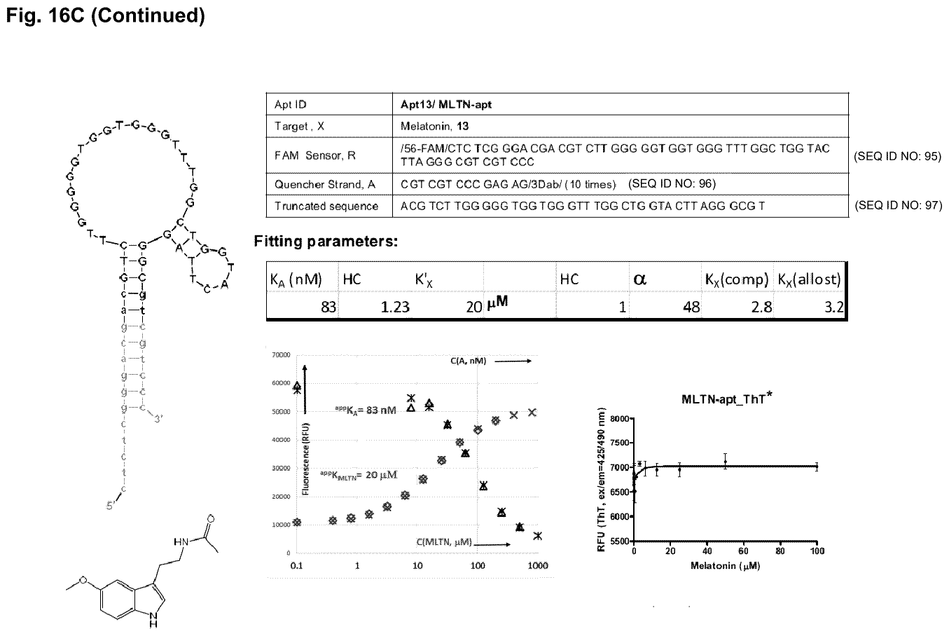

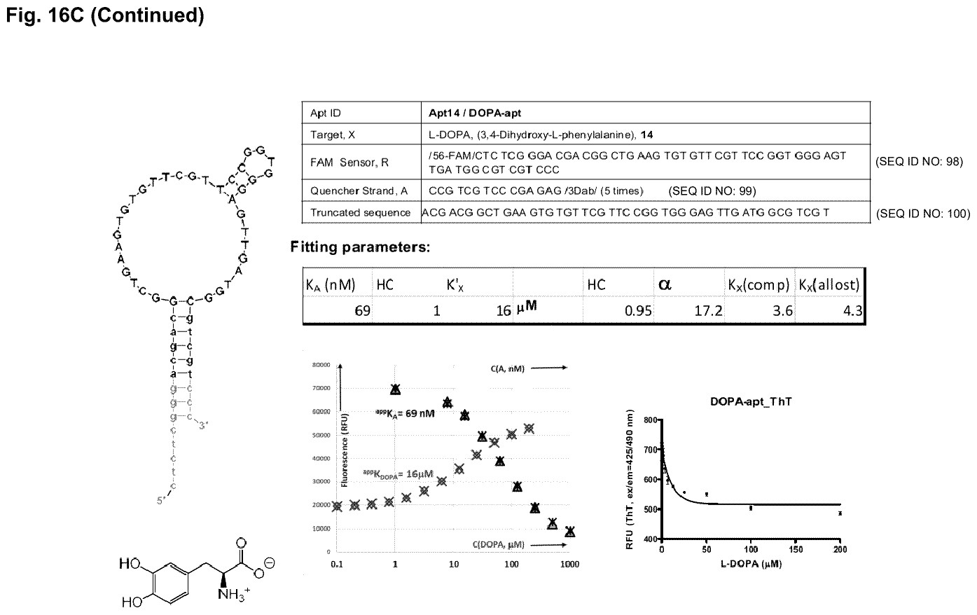

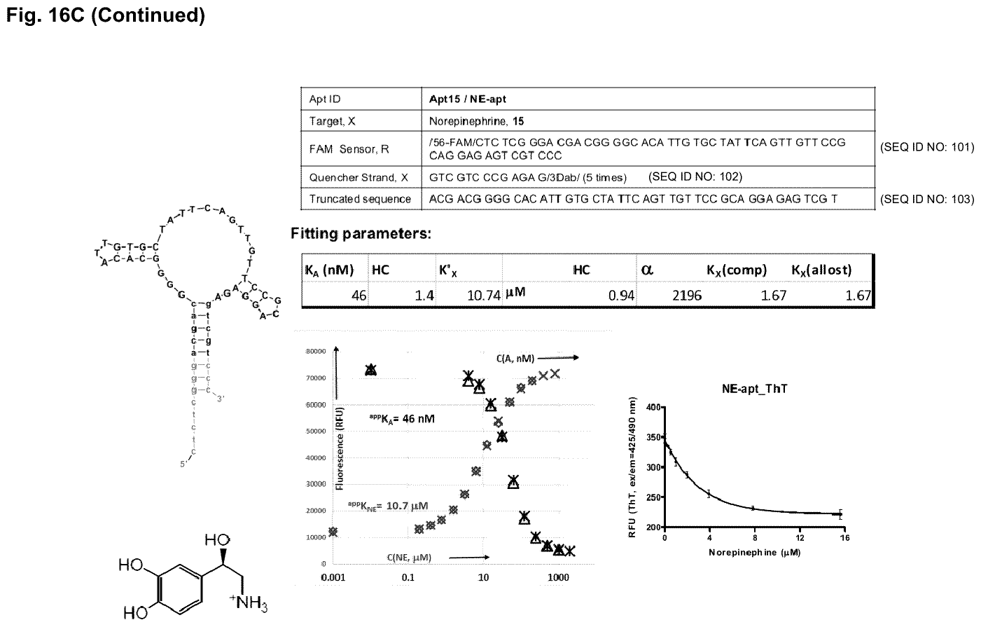

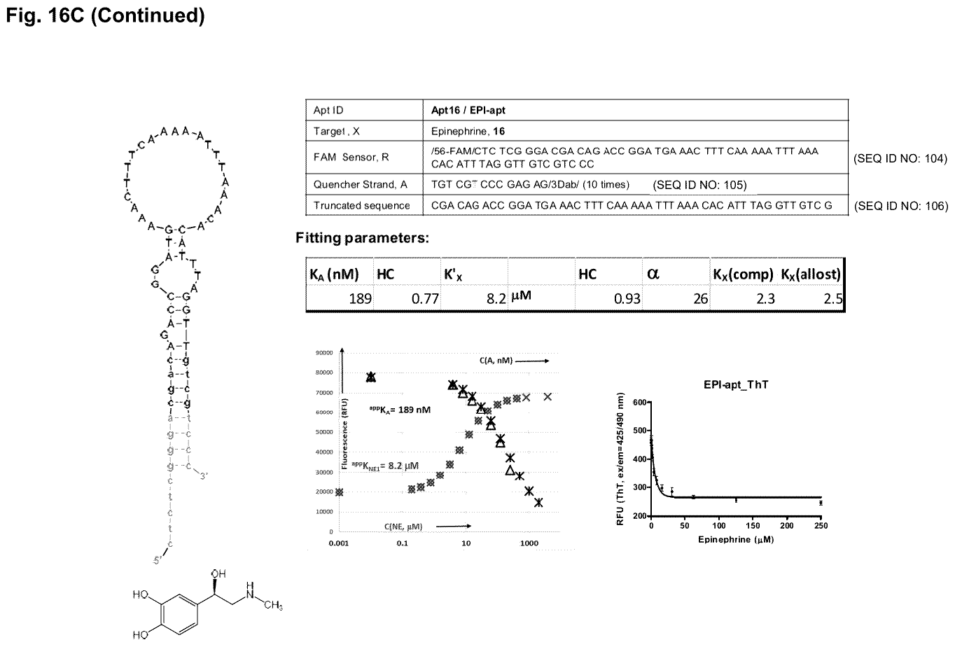

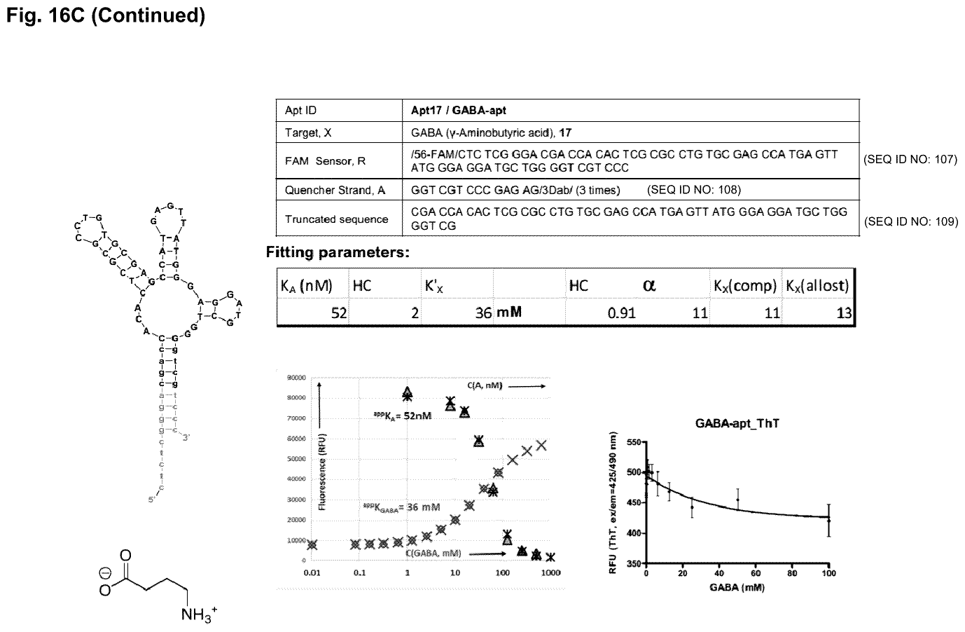

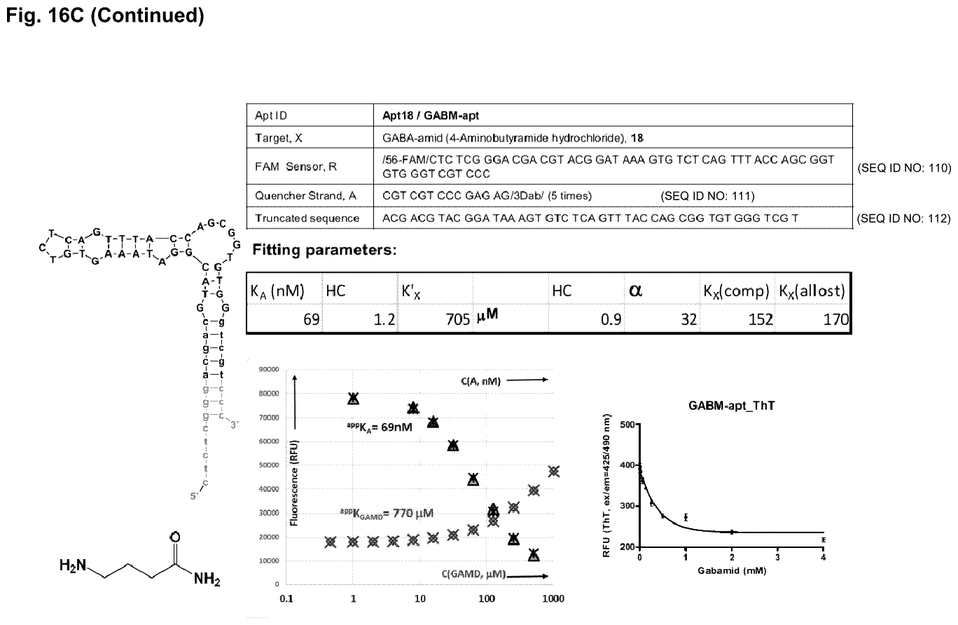

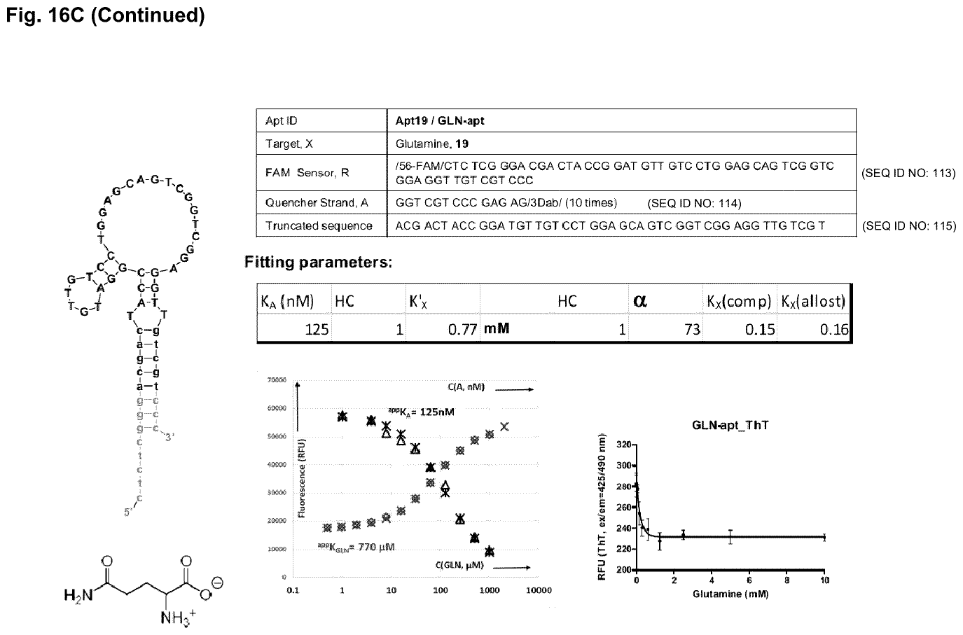

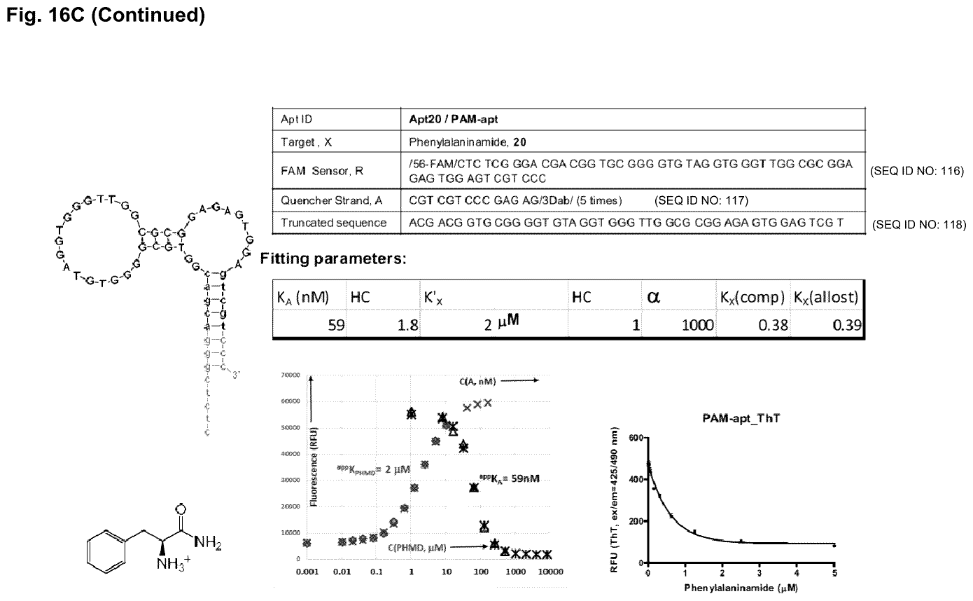

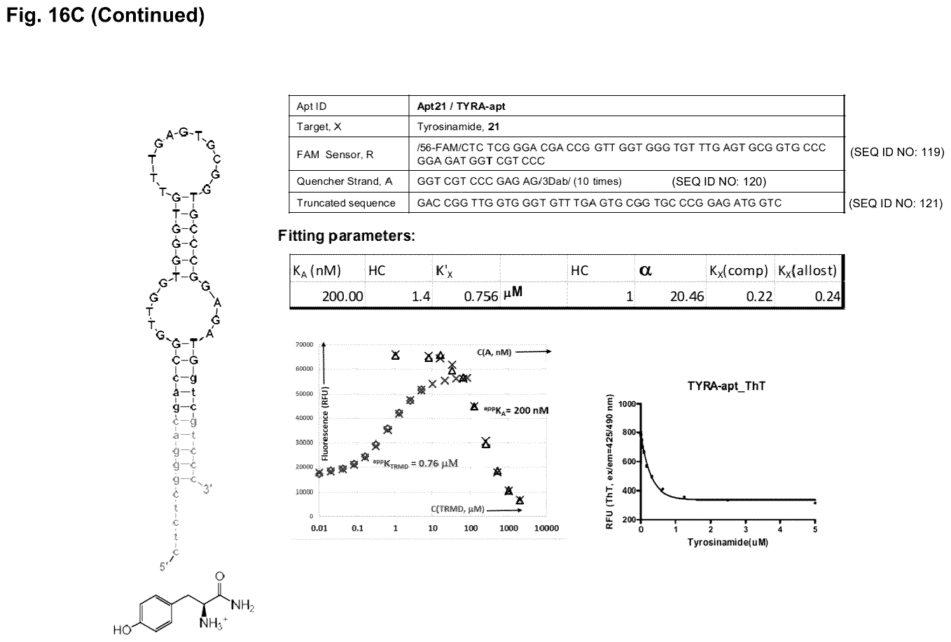

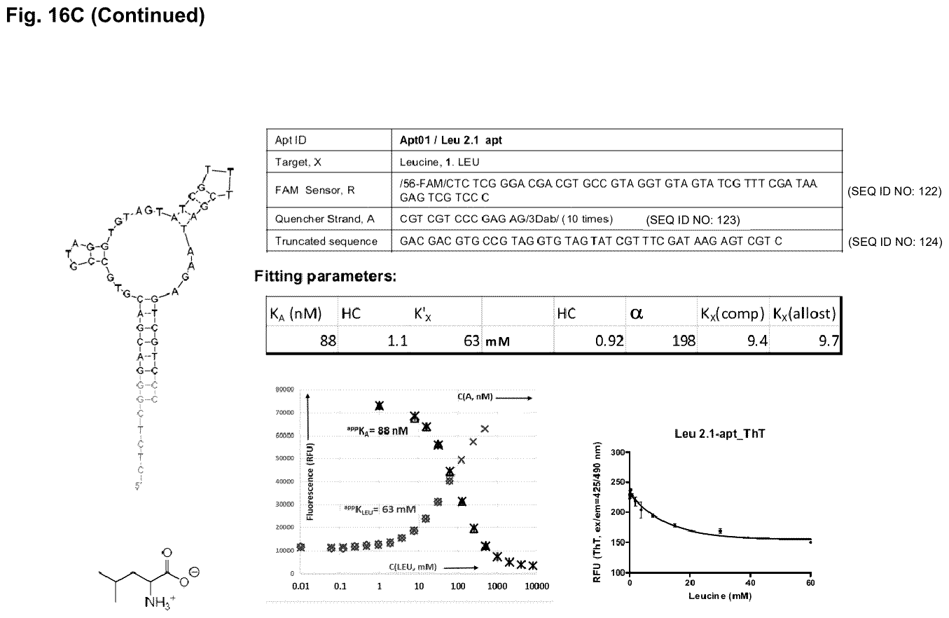

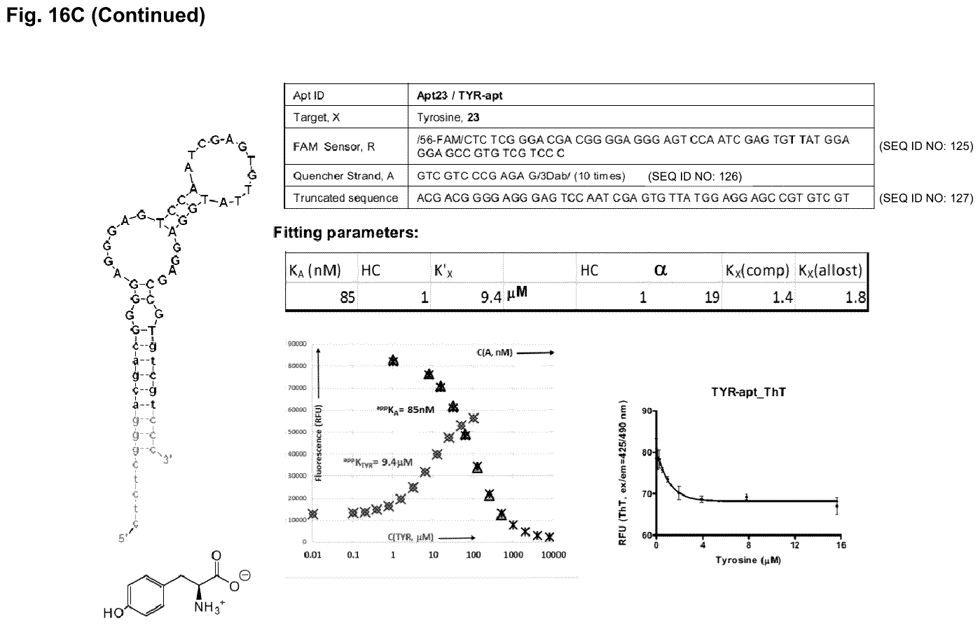

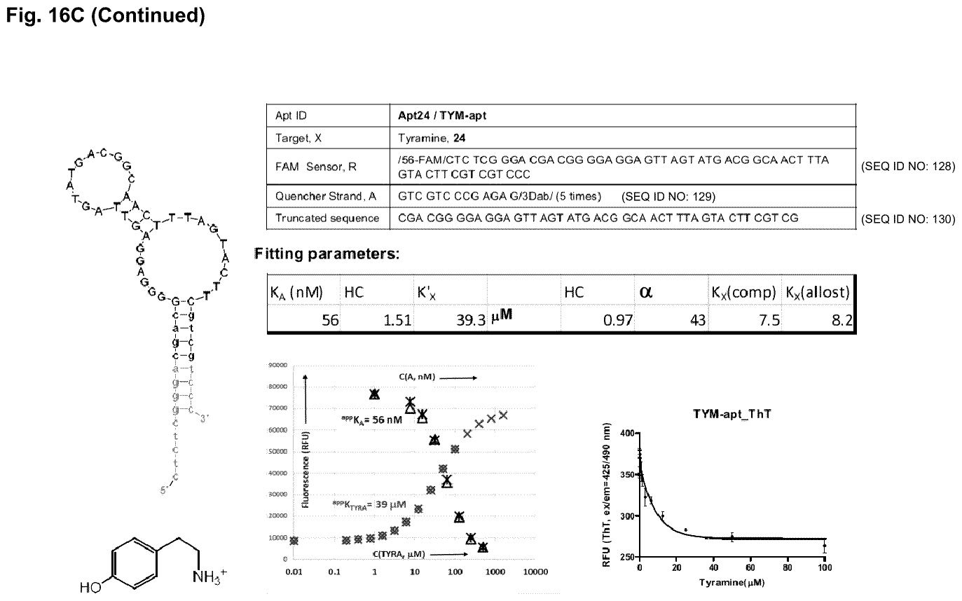

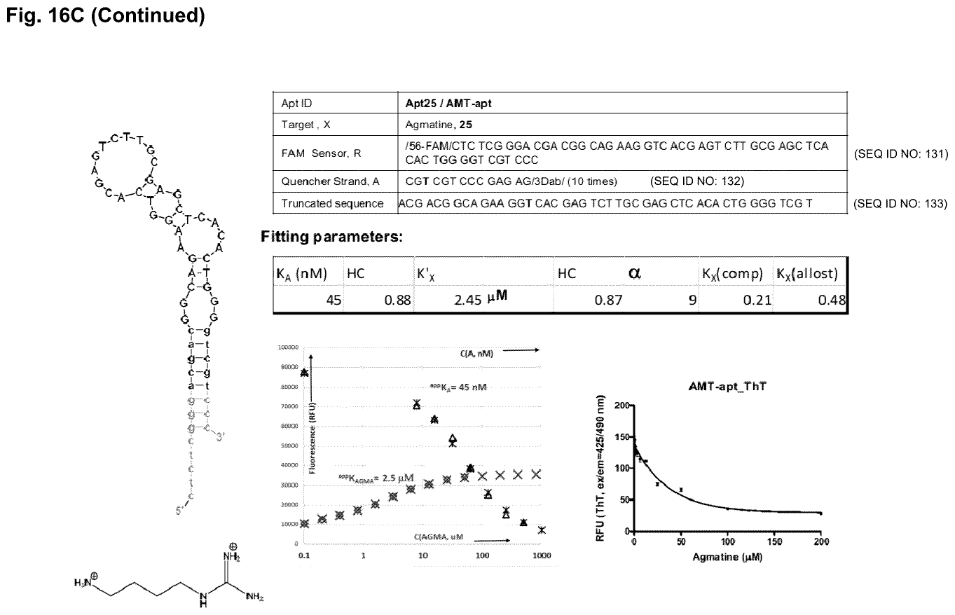

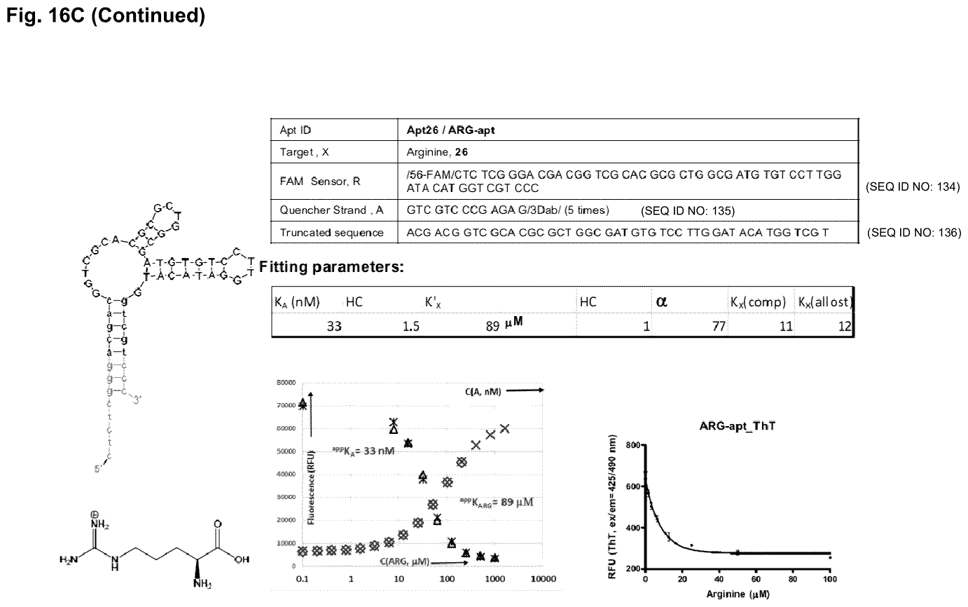

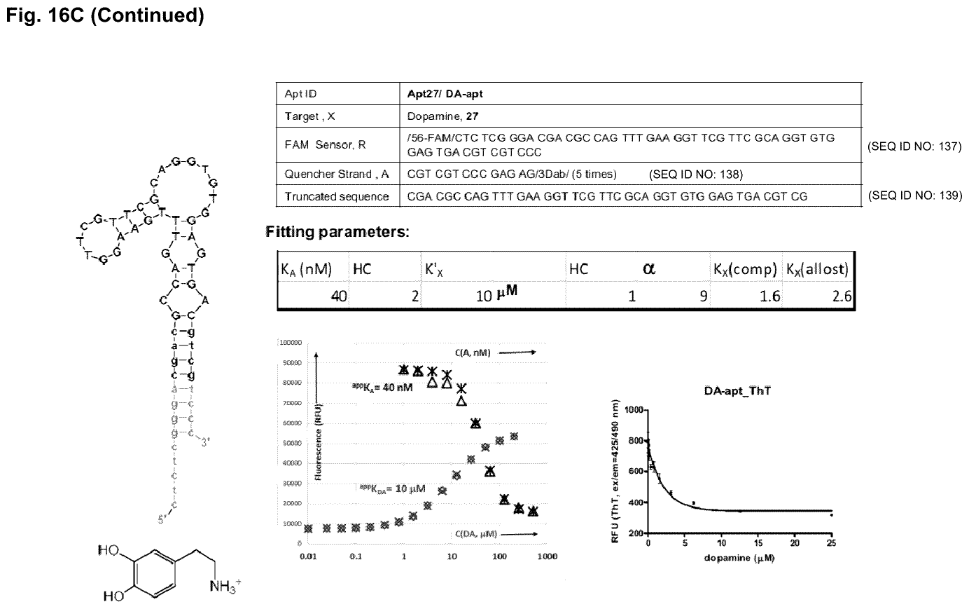

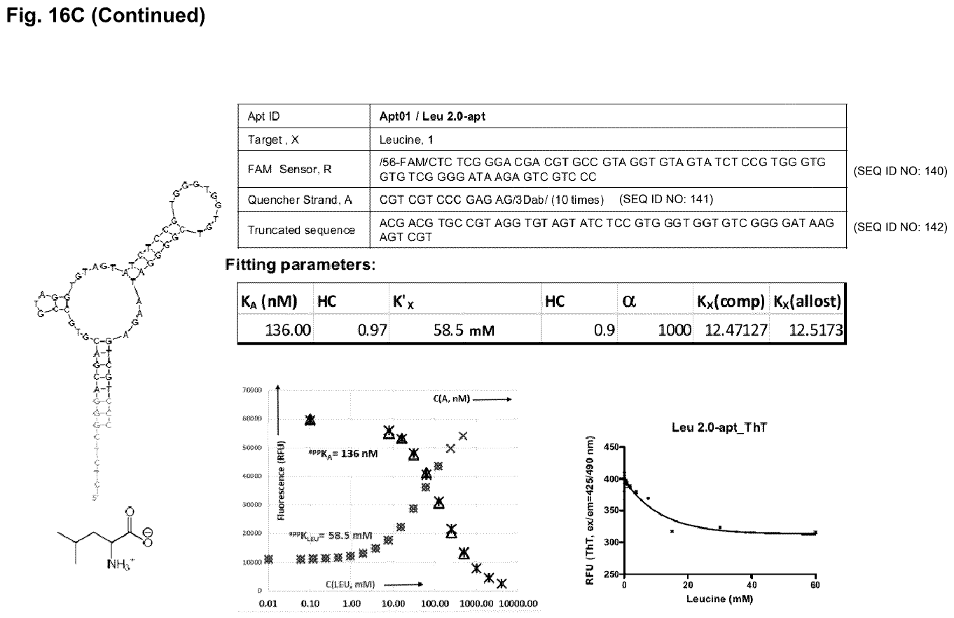

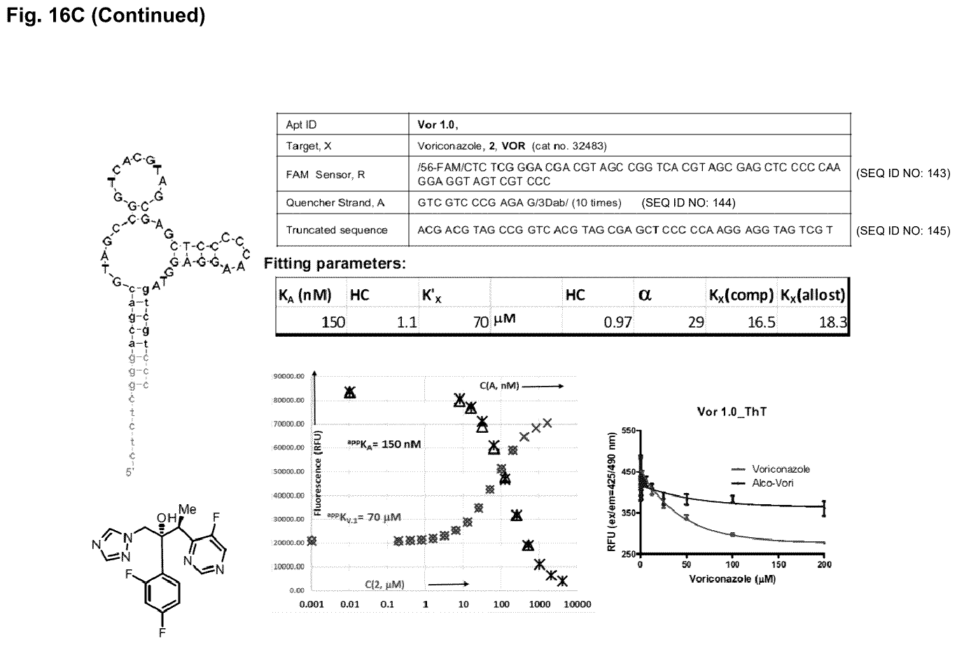

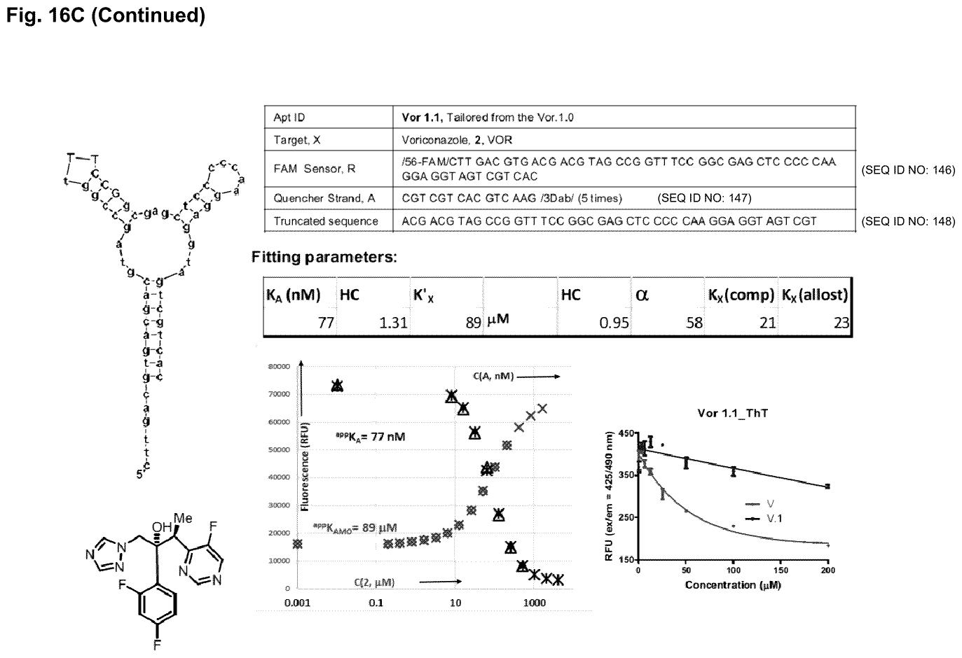

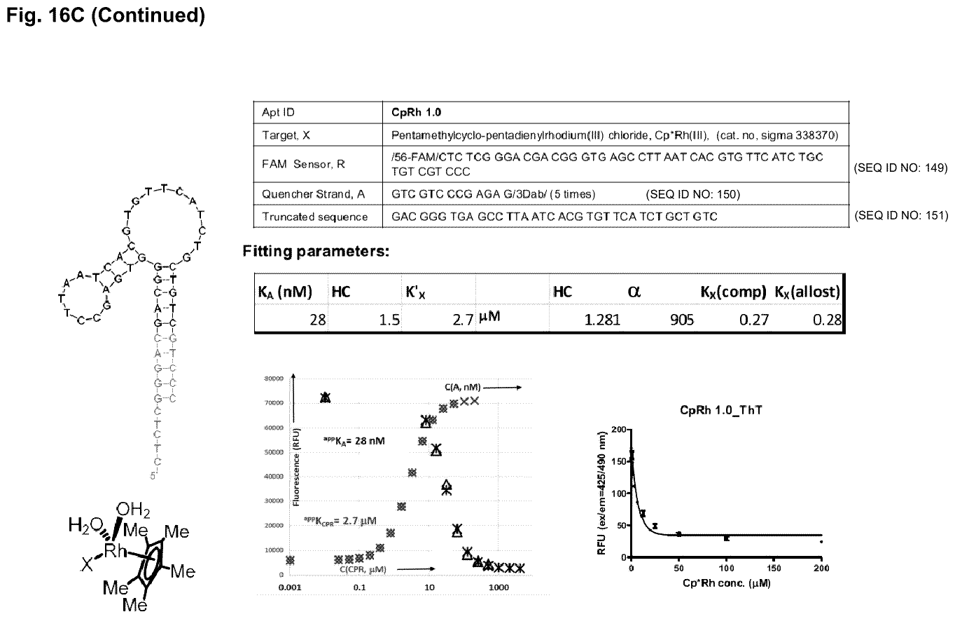

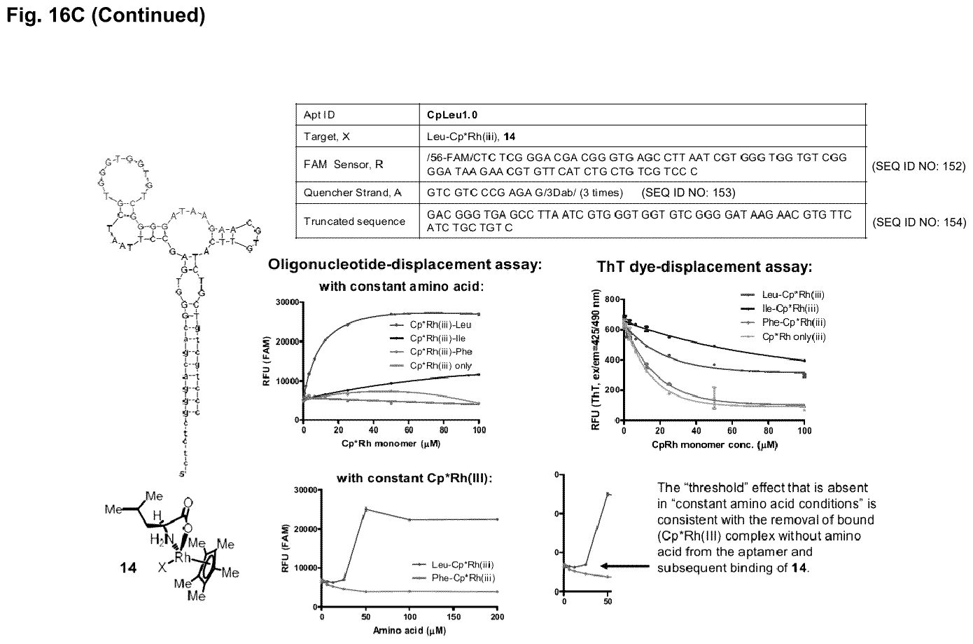

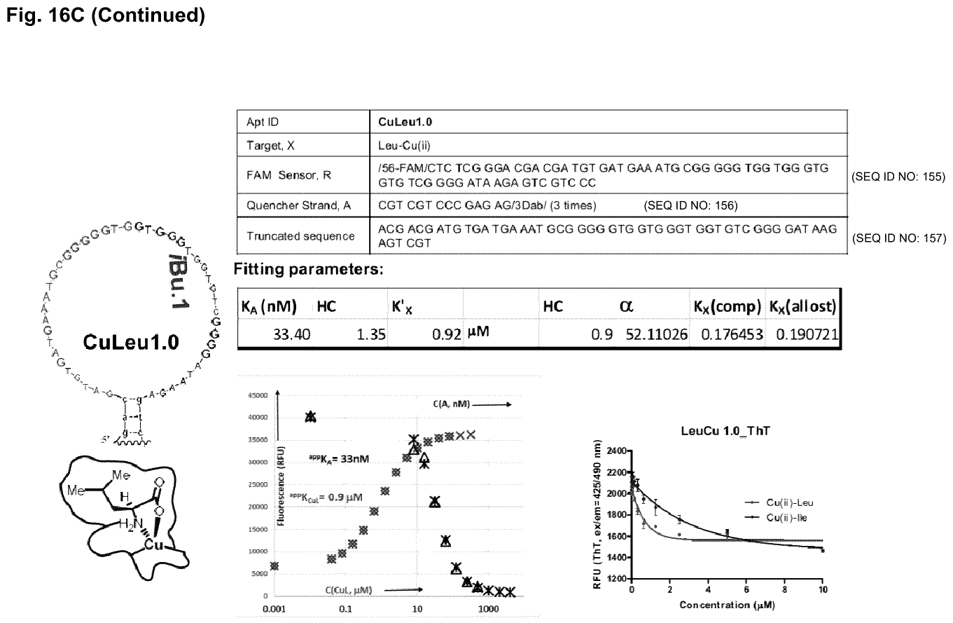

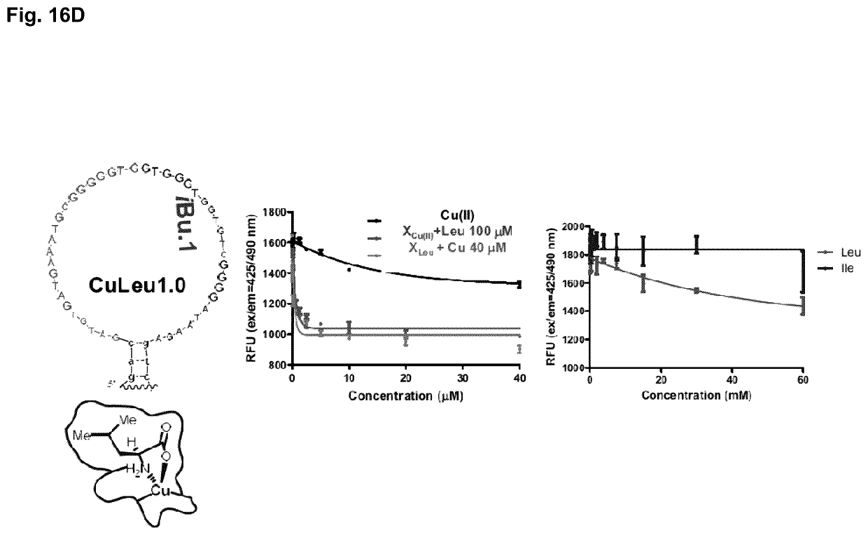

The aptamer candidates selected via ThT screening were further characterized using a fluorescence displacement assay. Fluorescein (FAM)-conjugated aptamers in their pre-truncated forms were used. Capture oligonucleotides were labeled with the fluorescence quencher dabcyl (Dab). Aptamers and capture strands for each target are listed in FIG. 16C. The FAM/Dab assay was carried out per the flowchart below in two steps.

A dissociation constant for the interaction between an aptamer and its capture strand was determined in the first step. This step was also used to determine the ratio of the dabcyl-labeled capture oligonucleotide to the FAM-labeled aptamer to be used in the next step. Each capture oligonucleotide was tested in serial dilutions starting from 500 nM with its corresponding aptamer at 50 nM.

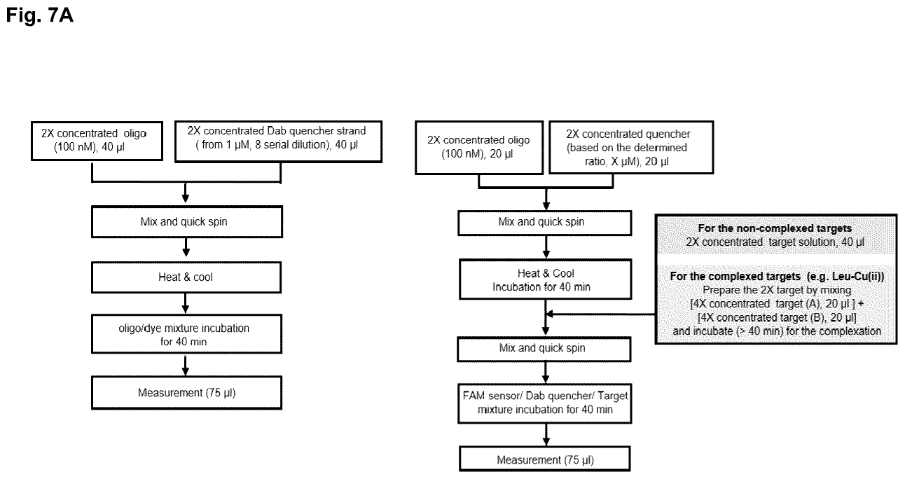

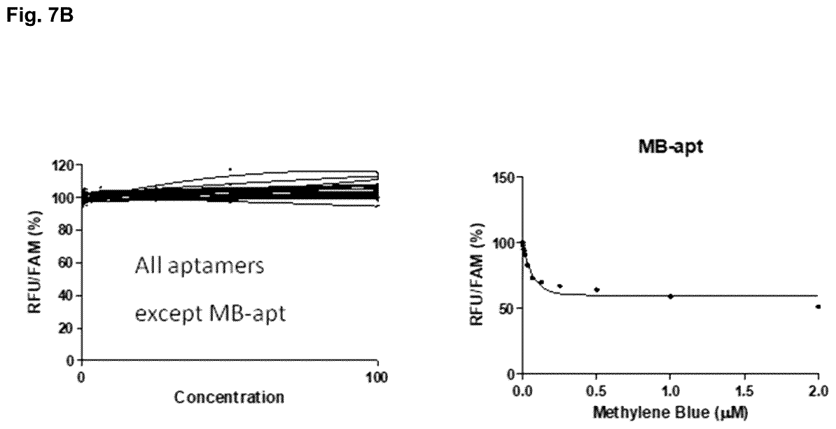

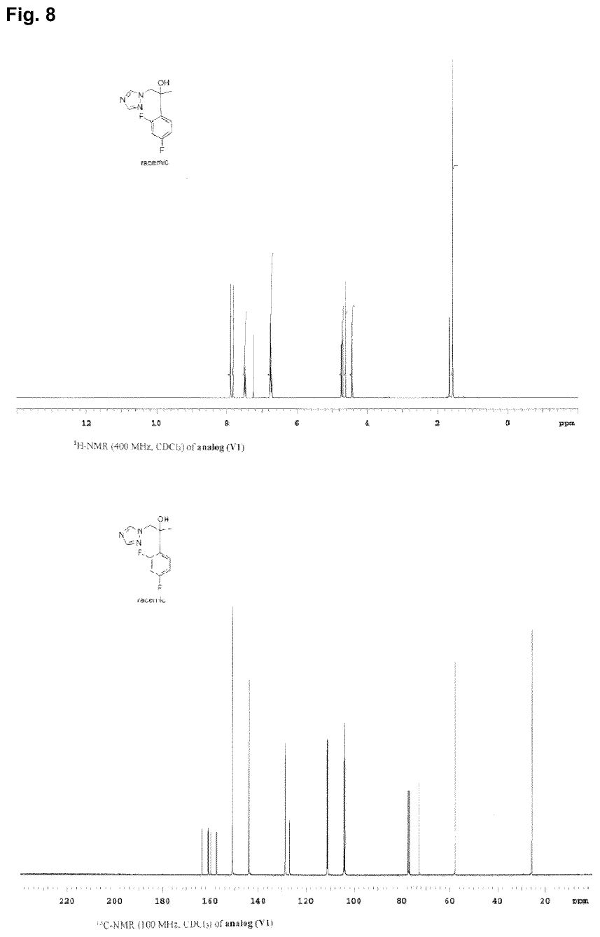

The ratio of aptamer to quenching oligonucleotide that produced ˜80-90% quenching was selected for the competition assay with each target. Target ratios were 3×, 5×, or 10× the aptamer concentration with the aptamer at 50 nM (See FIG. 16C for ratios for specific targets). Prior to determining fluorescence, each FAM-labeled aptamer and the corresponding dabcyl-labeled capture strand were mixed at the pre-determined ratio, placed in boiling water for 5 min, and allowed to cool to room temperature. Dilutions of the target solution were mixed with an equal volume of the oligonucleotide solution to obtain target-response curves. Solutions were incubated at room temperature for ˜40 min in the dark. Samples were analyzed in triplicate in 384-well black plates using a Victor II microplate reader (PerkinElmer, Waltham, MA) with FAM excitation/emission at 480 nm/525 nm. Controls were run with fluorescently-labeled aptamers in the presences of their targets but without quencher-labeled capture strands to determine the effects of targets on quenching. More information can be found in FIGS. 7A-7D.

### Synthesis of the Voriconazole Analog 2a

### Materials and Instrumentation

All solvents and reagents for chemical synthesis were purchased from commercial sources and used without further purification. Deuterated solvents were purchased from MilliporeSigma (St. Louis, MO). All reactions were carried out under nitrogen. Thin-layer chromatography was carried out on silica gel plates (pre-coated on glass, 0.25 mm thickness with fluorescent indicator UV₂₅₄). The ¹H and ¹³C-NMR spectra were recorded on a 400 MHz NMR spectrometer (Agilent Technologies, Santa Clara, CA) using CD₃Cl as the solvent. Chemical shifts are reported in parts per million (ppm) and referenced to residue solvent peaks (7.26 ppm for CDCl₃ for ¹H-NMR and 77.2 ppm for CDCl₃ for ¹³C-NMR). Abbreviations used in the NMR spectra are s=singlet, d=doublet, t=triplet, and m=multiplet. A single quadrupole liquid chromatograph mass spectrometer (LCMS-2020, Shimadzu Corp., Kyoto, Japan) equipped with a diode array detector and a C₁₈ column (SunFire, 50 mm×2.1 mm, 5 μm, Waters Corp., Milford, MA) was used to obtain low-resolution electrospray mass spectra and chromatograms.

### Synthesis of 2-(2,4-difluorophenyl)-1-(1H-1,2,4-triazol-1-yl)propan-2-ol)(analog (2a))

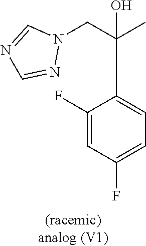 [SMILES: CC(O)(Cn1cncn1)c1ccc(F)cc1F]

Magnesium bromide ethyl etherate (2.80 g, 10.84 mmol, 2.4 eq) was added to a stirred solution of 1-(2,4-difluorophenyl)-2-(1H-1,2,4-triazol-1-yl) ethan-1-one (1.00 g, 4.48 mmol) in dichloromethane (anhydrous, 20 ml). The reaction mixture was stirred at room temperature for 1.5 h. The mixture was then cooled in an ice/water bath and a methylmagnesium bromide solution (3 M in ether, 4.1 ml, 12.3 mmol, 2.7 eq) was added dropwise. The resulting mixture was slowly warmed to room temperature and stirred for 3 d. The reaction was quenched with saturated ammonium chloride in water (20 ml). The product was extracted with dichloromethane (20 ml×3). The combined organic phases were concentrated. The residue was purified with column chromatography (silica gel, CH₂C₂/MeOH: 100:1 to 100:2). The desired product was obtained as a white solid, 480 mg (yield=44.8%). ¹H-NMR (400 MHz, CDCl₃): 7.92 (1H, s), 7.84 (1H, s), 7.54-7.47 (1s, m), 6.81-6.72 (2H, m), 4.73 (1H, d, J=14.0 Hz), 4.63 (1H, br. S), 4.45 (1H, d, J=14.4 Hz), 1.58 (3H, s). ¹³C-NMR (100 MHz, CDCl₃): 163.61, 163.48, 161.13, 161.01, 160.03, 159.91, 157.57, 157.45, 150.92, 143.97, 128.97, 128.91, 128.88, 128.82, 126.98, 126.94, 126.85, 126.81, 111.26, 111.23, 111.05, 111.02, 104.29, 104.04, 104.03, 103.77, 72.75, 72.71, 57.95, 57.90, 25.85, 25.82. LC-MS: calc. for C₁₁H₁₂F₂N₃O [M+H]⁺: m/z=204.09; found: 240.0. HPLC: C₁₈ column (SunFire, 2.1×50 mm, 5 μm); flow rate=0.2 mL/min; mobile phase: H₂O (with 0.1% formic acid/B (B=MeCN with 0.1% formic acid, B-gradient (started with 8%, increased to 100% in 15 min, keep 100% for 3 min, decreased to 5% in 2 min)); 20 min; RT=8.14 min; 91.1% @254 nm.

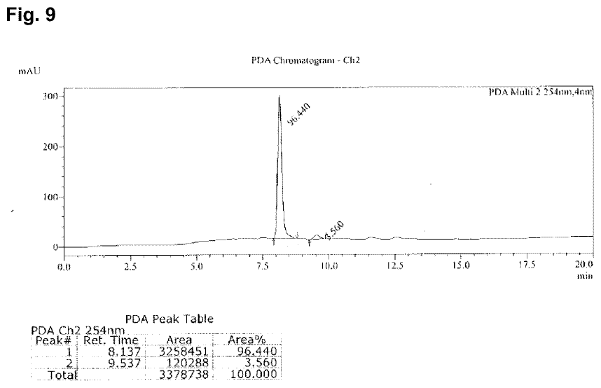

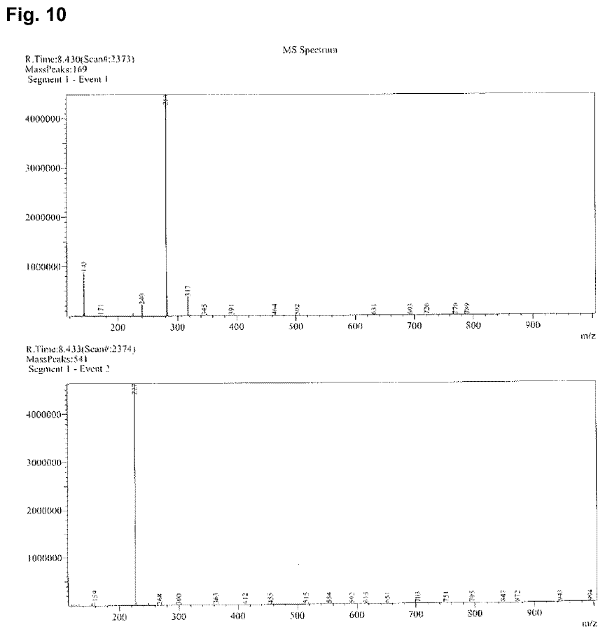

NMR analysis, HPLC chromatogram, and mass spectrum data for analog 2a can be found in FIGS. 8-10.

### Isothermal Titration Calorimetry

Aptamer absorbance ratios were determined (260 nm/280 nm<1.8) to confirm DNA purity prior to ITC experiments, which were performed using a MicroCal iTC200 isothermal titration calorimeter (Malvern, Worcestershire, UK). Aptamers were first heated to 95° C. for 5 min and slowly cooled to room temperature to reduce intermolecular interactions and facilitate the formation of native secondary structures. The reference cell contained 1×PBS with 2 mM MgCl₂ (300 μL). The sample cell contained the aptamer (300 μL, 5 μM) also in 1×PBS with 2 mM MgCl₂. The target (50 μM) in the same buffer was titrated into the sample cell using successive 1.5 μL injections until saturating changes in heat were observed.

Areas under the curve were integrated to calculate enthalpy changes for each target injection (kcal/mol). The molar ratio vs. the enthalpy of each injection was plotted to give ITC binding curves. Origin software was used to fit the titration curves to a one-site binding model. The slope of the isotherm gives the association constant. Its inverse is the dissociation constant, which was used to calculate ΔG:

$$Δ G = RT ln ( {K}_{D} )$$

### Example 2

### Displacement Assay Rationale, K_{D} Calculations, and ITC Data Introduction

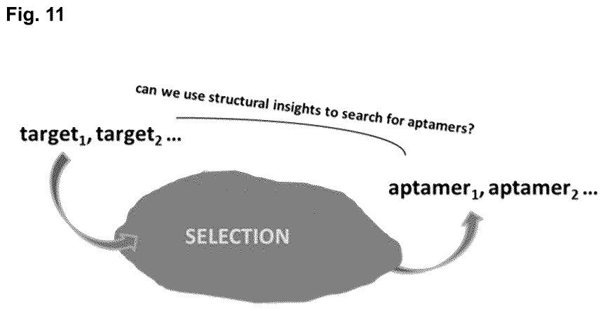

Our approach is to view aptamer selection as a black box with the stem-loop library, here N₃₆, as a constant, and thus, an integral part of the black box (FIG. 11). A target and its “winning” aptamer(s) are input and output(s), respectively. We ask, “Is there a connection between target structure and output, and if so, can this connection be used to search for aptamers, when a selection fails to produce outputs or if outputs are unsatisfactory?”

For the purpose of this work, we limit ourselves to the relationship between target structures and outcomes from the perspective of organic chemistry. Other approaches using the same set of aptamers are possible, such as focusing on informational content, mutagenesis, the structure space of N₃₆ stem-loop oligonucleotides, the impact of capture sequences, selectivity, or other characteristics of targets (e.g., numbers of specific bonds or Connolly accessible surfaces). These approaches, while interesting, are beyond the current scope.

We need a parameter through which we can quantify the impact of controlled changes in target structures on outputs. This parameter should be able to be consistently applied to a large set of aptamers with K_{D} values for their targets spanning three orders of magnitude (˜10⁻⁸-10⁻⁵ M) and should reflect anything that impacts selections, known and unknown, while ignoring all interactions irrelevant for selections. The parameter should be as parsimonious as possible and should be calculated under the same assumptions for all aptamers (to avoid cherry-picking and various biases). The results obtained using that parameter should pass tests as explained herein and be fully consistent with chemical intuition.

The fluorescence displacement assay, as studied in similar form and in depth by Li's group (14), with a capture oligonucleotide and an aptamer as it comes out of selection, is suitable for obtaining that parameter, because this type of displacement assay directly reflects equilibria and selection pressures within the column affinity domain in a way that is not replicated by other methods (e.g., isothermal titration calorimetry (ITC), see below).

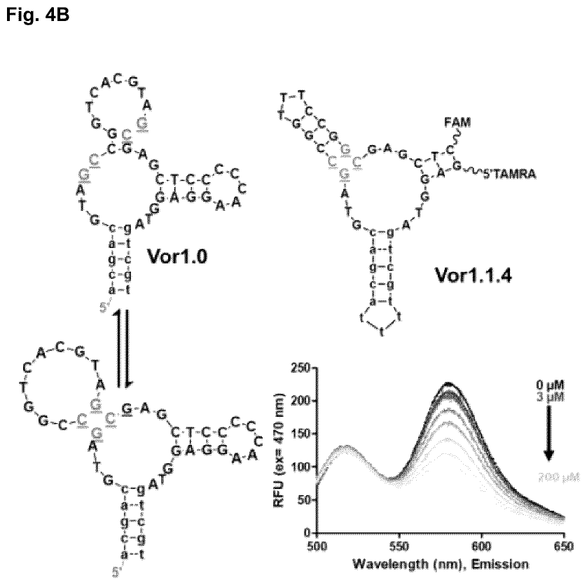

There are three primary components in the fluorescence displacement assay. They are a fluorescently labeled aptameric receptor (R), a partially complementary capture oligonucleotide labeled with a quencher (A), and a target (ligand) for the aptamer (X). These components are used in the same forms as those used in selections except that R and A are labeled with a fluorescent reporter and a quencher, respectively, and with the assay parameter [A] adjusted to achieve similar levels of quenching across different aptamers. We investigated 27 aptamers as “data points” rather than as biosensor components that need to be optimized (which is beyond the scope of the current work). For biosensors, both aptamers and quenchers can be optimized (see, e.g., FIG. 4D for the voriconazole aptamer).

In its simplest form, the fluorescence displacement assay considers aptameric receptor/capture strand hybridization in the absence of target and subsequent target concentration-dependent capture strand displacement. The equilibria governing hybridization and displacement are oligonucleotide specific. Hybridization positions the capture-strand quencher (dabcyl) next to the aptamer fluorophore (fluorescein, FAM) with all secondary interactions represented in the primary equilibria.

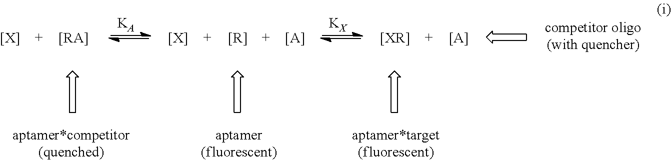

The fluorescence displacement assay consists of two experimental steps. First, we obtain K_{A}, which governs the equilibrium between R and A (the “quenching curve”). Second, we obtain the apparent equilibrium constant, K′_{X}, which governs the interactions of R with X in the presence of a constant concentration of A. This is a “displacement assay”, where the target concentration is titrated while the concomitant increase in fluorescence is measured.

With quenching conditions kept relatively similar (i.e., between 80-90% of the fluorescence of R on its own, without A), the value of K′_{X} (the half-point in the equilibrium, X_{50%}) and the change in free energy associated with target addition are characteristic of targets in the context of selections. By comparing aptamer-target free energies, we can extract the contributions of individual target functional groups. To calculate actual K_{X} values, which are affinities normalized across interactions with capture oligonucleotides, we have a choice of several models of increasing complexity.

### Obtaining the Dissociation Constant (K_{A}) for an Aptamer-Quencher Interaction

In our assay, K_{A} is derived from a traditional quenching assay, as in our previously reported studies (5, 6). Here, we perform a four-parameter logistic (4PL) curve fitting (e.g., at Quest Graph™ Four Parameter Logistic (4PL) Curve Calculator. AAT Bioquest, Inc., 4 Jan. 2023) to obtain Bₘₐₓ (“no quenching”). At this point, it is important to do a reality check, such that the Bₘₐₓ value is not allowed to be more than about 2% higher than the fluorescence of the aptamer alone or of the aptamer in the presence of maximal target concentration.

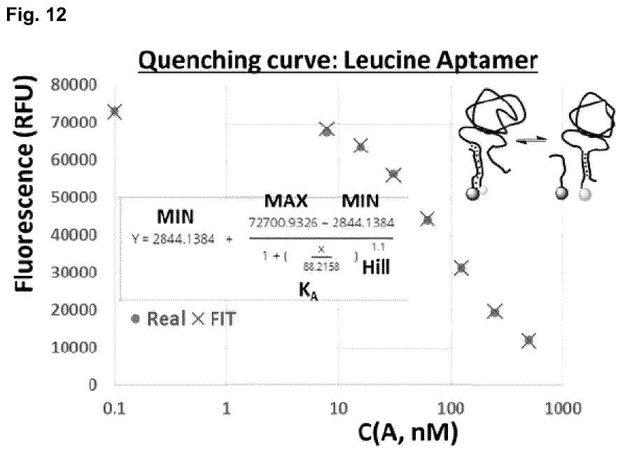

The half-maximal value of quenching, K_{A}, is at one half of a, the value in the presence of an infinite amount of capture oligonucleotide with quencher. Again, a reality check is necessary, such that the total quenching should not be below of about 2% of the fluorescence of the aptameric receptor alone. An example using leucine aptamer is shown in FIG. 12.

In the present disclosure, we use all aptamers as they come from selections with no attempts to optimize stems or displacement for analytical purposes. Because library capture, while the column is washed without target, is one of the applied evolutionary pressures, we expect that those aptamers that bind to more than one capture strand oligonucleotide, and still can be released upon exposure to the target, will be evolutionarily favored. Indeed, we observed that many of our targets will have interactions with more than one capture oligonucleotide in solution, with Hill coefficients varying between 0.85-2.66. Importantly, for example, numbers such as 1.8 do not mean that we have 1.8 capture oligonucleotides, but that, for example two oligonucleotides mildly interfere with each other. This is not proof, not even an indication, that these are happening on the column as well, because of the steric constraints on the capture oligonucleotides. Additional complication that makes HC difficult to interpret is that all our values are composite of microscopic constants in which aptamer and quencher are at various stages of hybridization, which have different affinities.

We always perform an additional reality check to look for a large discrepancy in fit for the region of the quenching curve between 20-80%. If necessary, we adjust parameters manually to obtain a more realistic curve fit. The value, K_{A} for each aptamer together with the sequences of R and A appear in FIG. 16C.

### Obtaining the Apparent Dissociation Constant (K′_{X}) for an Aptamer-Target Interaction

In our assay, K′_{X} is derived from a competition assay in which the target (X) is titrated while the displacement of a complementary capture oligonucleotide labeled with dabcyl (A) from the aptamer labeled with fluorescein (R) is observed. We again perform four-parameter logistic (4PL) curve fitting to obtain Bₘₐₓ (the “maximum release”), the half-maximal value of release, K′_{X} or X_{50%}, a minimal value (i.e., a starting point or value in the presence of an infinite amount of capture-Dab/aptamer-FAM), and a Hill coefficient. The latter parameter for all aptamers in our main set is always fitted to be between 0.85-1.25 and is treated as a 1:1 binding between X and R. Apparently, even if two target molecules bind to one aptamer molecule, one target molecule is predominant in quenching (or aptamer release from the column). Of note, in PRISM, this is “log(agonist) vs. response—Variable slope”.

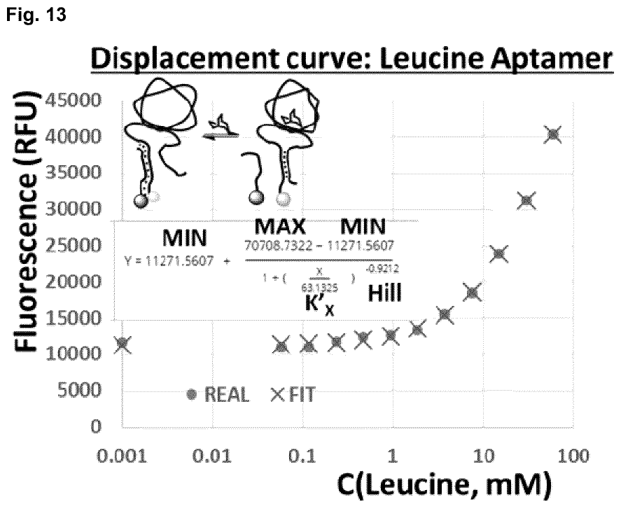

To ensure that, after we determine all K′x and Kx values, we calculate average contribution of oligonucleotide to free energy of displacement, and correct all values to it as if assay conditions are absolutely identical. While this protocol is not necessary for most situations, it can help us focus only on data that show significant difference with and without that correction. An example using leucine aptamer is shown in FIG. 13.

### Allostery to Model Capture Strand-Aptamer-Target Interactions

We can calculate all constants using either competitive (which we used in the past) or allosteric model (which we present here). Which model will use will not have practical impact on further use of aptamers, but we provide it as a starting point for future work. We base this analysis on Ehlert, 1987 (18) where the basic equations and terminology are as follows:

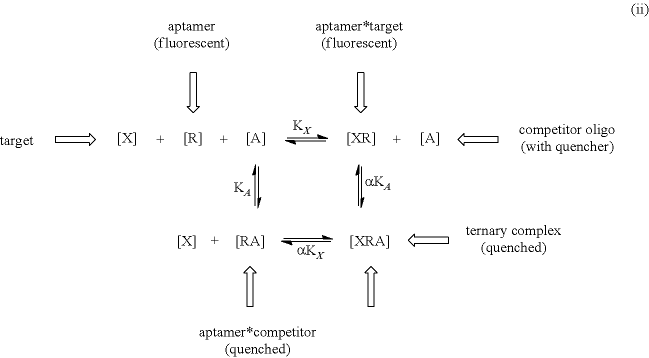

In this scheme:

The K_{A} is the dissociation constant for the interaction between a fluorescent aptamer and a complementary oligonucleotide labeled with a quencher.

The K_{X} is the dissociation constant for the interaction between a fluorescent aptamer and its target (small molecule ligand).

The value α denotes a factor by which the presence of A or X increases or decreases Kₓ or K_{A}, respectively (the impact is reciprocal, and by definition, cooperative). At high concentrations, the target becomes fully competitive (i.e., the target fully displaces the quencher-labeled capture strand). In this case, the equation below is reduced to a simpler form.

Ehlert derives the following equation to describe the binding of X in the presence of A:

$$Y = \frac{[ X ] \cdot [ {R}_{T} ]}{[ X ] + {K}_{X}^{′}} ; where : {K}_{X}^{′} = {K}_{X} \cdot ( \frac{{K}_{A} + [ A ]}{{K}_{A} + \frac{[ A ]}{α}} ) \qquad ( iii )$$

In this equation:

The value Y is aptamer (reporter) bound to ligand X ([Y]=[XR]+[XRA]).

The K_{X}′ value is the apparent dissociation constant of X (i.e., estimated by the target concentration at half-maximal displacement).

The RT denotes the total receptor concentration. In our case, RT is the aptamer concentration added to the solution.

We apply conditions described by Ehlert (p. 189), “It is often more economical to estimate the binding parameters of a nonlabelled drug by measuring the binding of a fixed concentration of a radioligand in the presence of various concentrations of the nonlabelled drug”.

All K_{A} values are in nM and all [A]_{T} (total concentration of competitor) are between 150-500 nM with similar quenching levels. These were fixed at such levels independently of this project, i.e., during initial characterization of our set.

Obtaining the α Factor from K_{A} at Saturating Target Concentrations

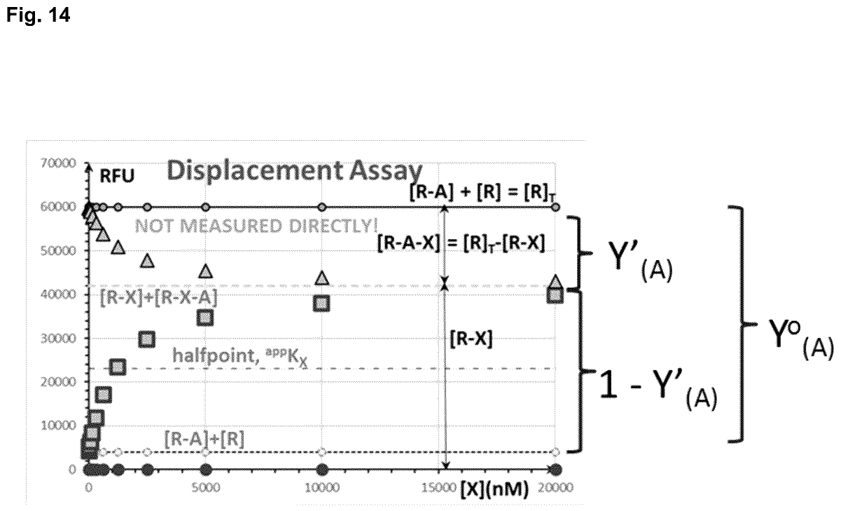

The factor α is, per Ehlert, estimated at saturating concentrations of X, using the following formula (p. 189):

$$α = ( {K}_{A}^{} + [ A ] * ( 1 - {Y}^{′} ) / ( {K}_{A}^{} * {Y}^{′} ) \qquad ( iv )$$

Where [A] is calculated while accounting for the binding of more than one A to one R, if appropriate. The Y′ and (1−Y′) are fractions best visualized in FIG. 14, wherein Y⁰ is the occupancy (R−A) at a maximum concentration of X and no A, while Y′ and (1−Y′) are fractions of the occupied and unoccupied receptor by A, respectively, in the presence of a saturating concentration of X. The grey points are the changes in concentrations in all fractions that we do not measure (dark), while the red points are an increase in the fractions that we can observe as an increase in fluorescence (i.e., those that have activated fluorescein). The total Y fractions can be measured directly by measuring fluorescence at saturation (or any other) point.
Obtaining K_{X} from K′_{X} (^{app}K_{D}) and α

Traditional competitive antagonism occurs at a high value of α such that:

$${K}_{X}^{} = {K}_{X}^{′} / ( 1 + [ A ] / {K}_{A}^{} ) \qquad ( v )$$

At lower a, we observe allosteric antagonism:

$${K}_{X}^{′} = {K}_{X}^{′} / ( {K}_{A}^{} + [ A ] ) / ( {K}_{A}^{} + [ A ] / α ) \qquad ( vi )$$

Our values of α range from 3 to >100. Lower values of α typically result, in our case, in a 2-3-fold change in calculated affinity, which is negligible in the context of our limited conclusions.

If of interest, modeling the next level of complexity would require using microscopic binding constants, one or two capture strands, and one or two target molecules, and their individual impacts on fluorescence and quenching, as appropriate, for each aptamer. That level of complexity could be, for example, with some aptamers, pursued by combining the approaches used to generalize a Monod-Wyman-Changeux framework (45). The rigorous mathematical treatment of up to three interacting molecules, as well as a window into the complexities that could be encountered while studying binding equilibria of more than two bodies are provided by Siegel and colleagues (46), while extending this approach to ITC experiments is possible (see below, and also (47)). However, these additional levels of complexity were deemed unlikely to be beneficial for our analysis, requiring individual special considerations and assumptions for each aptamer.

Calculating ΔG_{D} Values from ^{app}K_{D}

ΔG_{B} is defined in medicinal chemistry as energy that is invested in a system to dissociate ligand from its complex. ΔG_{D} energy that a system releases when ligand is added, and it has negative value. In FIG. 1B, we accordingly show ΔG_{D}=−RT ln(1/^{app}K_{D}) in formula, which is same as ΔG_{D}=RT ln(^{app}K_{D}).

This may cause some confusion, since we also defined free energy contribution of functional groups to displacement using letter B, not D, and they will have opposite signs. This complicates some language (favorable contribution becomes negative), but it all works out in absolutely the same manner regardless of this choice, as long as it is used consistently.

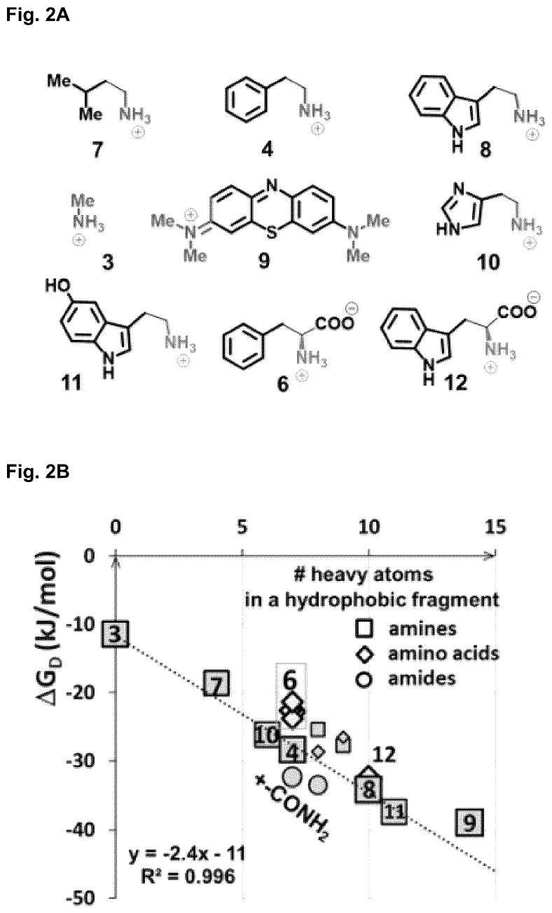

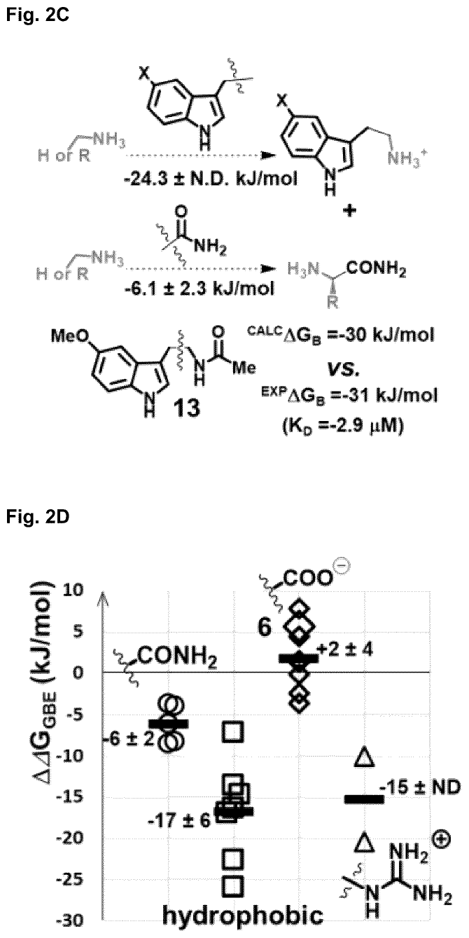

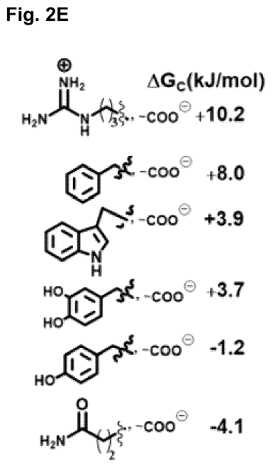

We use app K_{D} values to assess the impact of individual target functional groups on selections (FIG. 2), via calculating, and then subtracting, pairwise ΔG_{D} values. Because levels of quenching are relatively the same (i.e., contribution of the presence of oligonucleotide is approximately constant 3.5+/−0.7 kJ/mol if we calculate K_{X} using allosteric model, and then average them across all targets, see Note below), we can use ^{app}K_{D} values “as they are” to calculate functional group contributions. However, in some cases, when ΔΔG_{GBE} values are close to 0, of displacement energies are close, we want to avoid overinterpreting small differences. Thus, we introduce corrections, by adjusting ΔG_{D} value so each oligonucleotide contribution is exactly at 3.5 kJ/mol, dismissing any conclusions that would depend on this change either way. That is if contribution due to level of quenching is calculated to be exactly 3.0 kJ/mol, we add to value 0.5 kJ/mol to adjust it to 3.5 kJ/mol. This “brings” all competitor oligonucleotides aspects of assays at exactly the same level, and substitutes adjusting endlessly all assay conditions. Importantly, we avoid interpreting results.

Note: Explanation of correction factors and corrected ΔG_{D}

appΔGD is calculated from X_{50%}, a half-point of displacement assay. We could directly subtract two values to obtain functional group contribution to binding energy, however, that would assume that for each case in the pair contributions of oligonucleotides are identical (i.e., that aptamers with fluorescein are quenched to the same level). We can assess this assumption by taking an average (ΔG_{D}−ΔG_{B}) values for, say, ΔG_{B} calculated through either allosteric or competitive model (see Table 2). We obtain, e.g., for allosteric model, that average contribution of oligonucleotide is 3.5+/−0.7 kJ/mol, with extreme range from 2.8 to 5.9 kJ/mol (ammonia and arginine). This warns us not to over-interpret close data points, particularly when subtracting pairs that are at extremes of this distribution.

To minimize impact of uneven contributions of the oligonucleotide with dabcyl to quenching, we adjust each data point (add or subtract) by the value that would bring it to the same level, of 3.5 kJ/mol. As a caution, we avoid making any statements about data points that would change significantly relative to other data points. Effectively, we used this process to eliminate those data points at which this uncertainty would impact our conclusion. For example, impact on these data points are minimal:

| | | | Allosteric | | |
|---|---|---|---|---|---|
| Name | appDGD | DGB(allosteric) | Difference | Factor | all. corDGD |
| histamine | −26.5 | −29.7 [symbol] | 3.2 | 0.3 | −26.2 |
| phenylethylamine | −28.8 | −32.0 [symbol] | 3.1 | 0.4 | −28.4 |
| tyramine | −24.7 | −28.6 [symbol] | 3.9 | −0.3 | −26.0 |
| | [symbol] | [symbol] | [symbol] | [symbol] | [symbol] |
| | calculated | calculated from | difference | Difference | Presented |
| | from X_{50%}, | X_{50%}, and K_{A} | (average | from average | |
| | | (oligonucleotide) | is 3.5) | | |
| | | using allosteric | | | |
| | | model | | | |

[symbol]

indicates data missing or illegible when filed

### Representative ITC Experiments and Some Notes of Caution.

Isothermal titration calorimetry (ITC), when possible, is widely accepted as the gold standard for characterizing the affinity of ligands for their receptors. We carried out ITC on representative aptamer-target pairs as an additional method of determining K_{D} and hence, ΔG. However, several issues complicated ITC for use with small aptameric receptors binding to small targets.

Our main goal is to define the impact of a target and its functional groups on our ability to select aptamers, which is not the same as defining aptamer-target binding by itself. A major difference lies in the additional equilibrium established with an aptamer in the presence of a capture oligonucleotide, which induces new conformations in the aptamer itself and the aptamer in the presence of the target. These additional equilibria, in principle, could be investigated by ITC by studying the binding of a capture oligonucleotide and an aptamer, and then trying to reproduce the selection pressure that arises from having all three species-aptamer, capture strand, and target-present at the same time. The displacement assay, by design, reproduces in a much more straightforward manner the capture oligonucleotide-aptamer equilibrium and the subsequent impact of a target on this equilibrium replicating the dominant thermodynamic driving forces to release aptamer candidates from the column in the aptamer selection process. Further, the displacement assay focuses observable interactions strictly on those that cause an increase in fluorescence, which is a result of a chemical process identical to that which causes aptamer release from the column.

To obtain measurable changes in heat in ITC for small receptors and small ligands, even with microcalorimetry, we needed to use higher aptamer concentrations (5 μM for ITC vs. 50 nM for the displacement assay) and thus, higher target concentrations. Some of our targets have limited solubility and/or aggregate in selection buffers. Moreover, while we heated and cooled aptamer solutions prior to ITC, aptamer-aptamer interactions may still occur at the aptamer concentrations required for high signal-to-noise in isothermal calorimetry titrations. Moreover, some targets (or target classes) show evidence of multi-site binding.

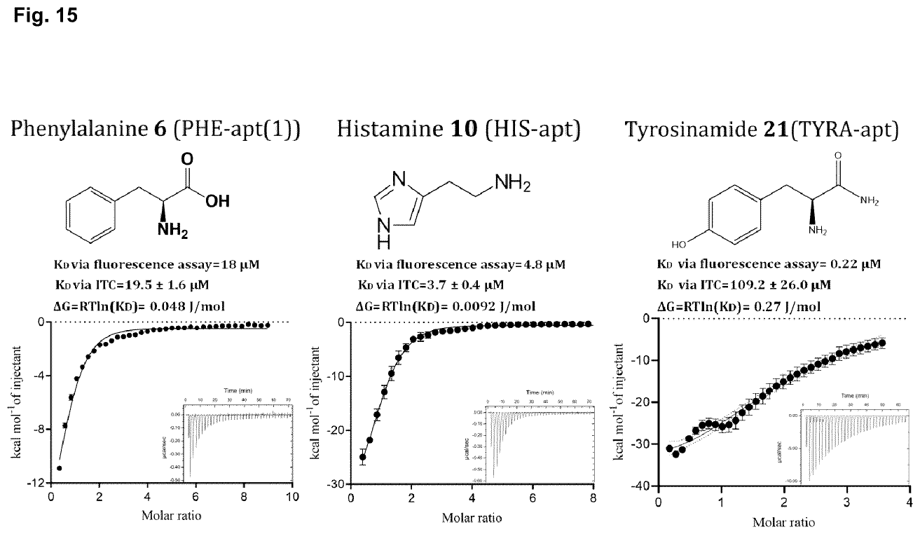

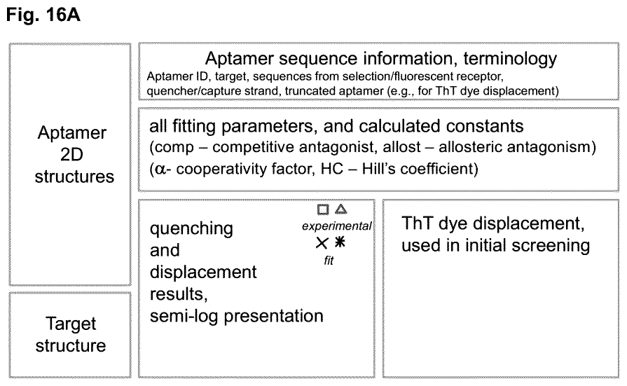

On the next page, we show ITC examples for three aptamer-target pairs representing different target classes (i.e., amines, amino acids, and α-amino amides) where targets were soluble, and the results were reproducible (FIG. 15). In the case of the amide, we observe a complication of multiple binding sites, which obviously does not impact release from the column, thus, pointing to another advantage of fluorescence displacement assay for the purposes of this study. Some other aptamer-target pairs had complications that prevented us from obtaining clear-cut dissociation constants and therefore, from using ITC across the entire set to describe the impact of target functional groups on selection. Thus, while ITC is not suitable for our purposes, it can certainly be used in the future to “shine light” on the box, together with more detailed structural studies of individual aptamers.

### Example 3

### Analysis of Free Energies of Oligonucleotide Displacement Across Related Targets

We amassed 27 aptamers, 23 of which were newly isolated through this work (Table 3). The new aptamers emerged directly from selections, without further optimization, identified as the highest affinity receptors targeting amines, amino acids, and their analogs. In the past, while working with individual aptamers, we focused on aptamer dissociation constants obtained by a fluorescence quenching assay that reported fluorescently labeled aptamer competition with a quencher-labeled capture oligonucleotide (FIGS. 1C, Table 1 and Table 2; the assay could be adapted to a model of allosteric antagonism to account for partial release upon binding (18)). To characterize impact of targets on selection outcomes, we instead needed to compare targets in their abilities to outcompete capture oligonucleotides. Thus, we focused on ^{app}K_{D} (a midpoint response or X_{50%}) of the displacement of oligonucleotide competitor that is used on affinity column during selection, which is related to the free energy of displacement, ΔG_{D}. In contrast to the free energy of binding (ΔG_{B}) obtained, e.g., by isothermal calorimetry, ΔG_{D} governs a comprehensive set of equilibria that impacts the release of aptamers from the column upon target addition. The difference between ΔG_{D} and ΔG_{B} is primarily in the contributions of the capture oligonucleotide present at equilibria.

TABLE 3
Target chemical list used for aptamer selection;
chemical catalog numbers are from Sigma-Aldrich
(MilliporeSigma), unless otherwise noted.

| # | Aptamer ID | Target Name | Cat. No. |
|---|---|---|---|
| 1 | Leu 2.1 apt | L-Leucine | L8000 |
| 2 | Vor-apt | Voriconazole | PZ0005 |
| 3 | MEA-apt | Methylamine (methylammonium | 8.06020 |
| | | chloride) | |
| 4 | PHEA-apt | 2-Phenylethylamine HCl | P6513 |
| 5 | GLY-apt | Glycine | BP381-1 |
| | | | (Fisher |
| | | | Scientific) |
| 6 | PHE-apt(1) | L-Phenylalanine | P5482 |
| 6 | PHE-apt(2) | | |
| 6 | PHE-apt(3) | | |
| 6 | PHE-apt(4) | | |
| 7 | MBA-apt | Methylbutylamine (1-amino-3 | 17773 |
| | | methylbutane) HCl | |
| 8 | TRPA-apt | Tryptamine HCl | 246557 |
| 9 | MB-apt | Methylene blue | M9140 |
| 10 | HIS-apt | Histamine 2HCl | H7250 |
| 11 | SRTN-apt | Serotonin HCl | H9523 |
| 12 | TRP-apt | L-Tryptophan | 93659 |
| 13 | MLTN-apt | Melatonin | 461326/M5250 |
| 14 | DOPA-apt | L-DOPA | D9628 |
| 15 | NE-apt | (−)-Norepinephrine | A7257 |
| 16 | EPI-apt | (−)-Epinephrine | E4250 |
| 17 | GABA-apt | GABA (g-aminobutyric acid) | A2129 |
| 18 | GABM-apt | GABA-amid (4-Aminobutyramide) | FA17704 |
| | | | (Biosynth) |
| 19 | GLN-apt | L-Glutamine | 49419 |
| 20 | PAM-apt | L-Phenylalaninamide | P1883 |
| 21 | TYRA-apt | L-Tyrosinamide | T3879 |
| 22 | GAM-apt | Glycinamide HCl | G6104 |
| 23 | TYR-apt | L-Tyrosine | T3754 |
| 24 | TYM-apt | Tyramine | T90344 |
| 25 | AMT-apt | Agmatine H₂SO₄ | A7127 |
| 26 | ARG-apt | L-Arginine | A8094 |
| 27 | DA-apt | Dopamine HCl | H8502 |
| 28 | Ammo-apt | Ammonia (ammonium chloride) | 254134 |

The targets (FIG. 16C) and their aptamers (FIG. 16C) were organized in 42 related pairs (FIG. 1D), with each pair differing by the addition of a single functional group or group transformation, e.g., methylamine (3) and phenylethylamine (4) differ by the addition of a seven-carbon benzyl group (FIG. 1D). We defined ΔΔG_{GBE} as the free energy difference related to the equilibria positions impacting the relative outcomes of two selections, attributable to the presence of the additional functional group or transformation. Further, we assume the portions of ΔG_{D} that govern equilibria unrelated to either target or capture oligonucleotide binding to be similar across all aptamers and that they will largely cancel each other when subtracting two ΔG_{D} values within pairs, which allows us to extract estimates of relative ΔΔG_{GBE} values (FIG. 1D). Related concepts on contributions to the free energy of binding associated with functional groups are often used for ligand optimization in medicinal chemistry (19, 20), where receptors are shared between targets. Two key assumptions, aside from nearly identical selection conditions, were needed to extend the concept of functional-group free-energy contributions to selections:

First, there are ˜10²¹ possible random 36-mers. In selections, we sample only ˜10¹⁴ of these sequences. Thus, in the absence of extraordinary luck, we do not isolate potentially unique, but only typical receptors (21, 22), which are examples of multiple sequences having similar affinities values broadly distributed over oligonucleotide space. This sparse sampling allows us to treat the properties of the isolated aptamers, represented here by the ‘best’ aptamer from each selection, as characteristic of the highly standardized selection conditions, libraries, and targets. Since selections for new and previously identified aptamers differed only in their targets, we attributed large changes in the properties of the aptamers to the impact of structural differences between targets, i.e., to specific functional groups.

Second, functional group contributions to selections can only be based on well-known non-covalent interactions (20, 23). Thus, as a first approximation, within a set of close analogs, we expect to be able to isolate additive effects. When we observe systematic non-additivities in thermodynamic cycles, for example, cooperativity (ΔG_{C}) as estimated through cycles of double replacements of functional groups (24), we can analyze non-additivities to generate hypotheses about barriers to aptamer isolation (FIG. 1E). Reciprocally, if correct in our assumptions, after initial selection failures, we can perform functional group analysis of targets to identify possible structural barriers leading to these failures and design selection protocols to improve our chances of isolating aptamers.

We performed the following three tests with the available aptamers to assess these assumptions. While each test individually was limited due to small sample sizes, together, they strongly supported our reasoning. First, we analyzed the four highest affinity aptamers for phenylalanine from four separate selections and obtained similar ΔG_{D} values (and estimated ΔG_{B} values), within <3 kJ of each other (FIG. 2B, Table 4). This is consistent with the affinity of winning aptamers being regularly distributed over oligonucleotide space, and thus, representing a reproducible property of selections. These findings suggest that large differences in target-related ΔG_{D} should reflect differences in functional groups and not different selections.

TABLE 4
Relationship between ΔG_{D} (calculated from ^{app}K_{D}, half point), ΔGB competitive and allosteric, correction factor (deviation
from average) to bring all oligonucleotide impacts on the same level, leading to corrected ΔG_{D}, which was used in presentation.

| (all ΔG values are in kJ/mol) | | | | | ΔG^{c}_{B} | ΔG^{a}_{B} | ΔG^{c}_{D-B} | ΔG^{a}_{D-B} | | Δ^{app}G^{a}_{D} |
|---|---|---|---|---|---|---|---|---|---|---|
| | appKD | Competitive | Adllosteric | Δ^{app}G_{D} | DGB(competi- | DGB(allo- | Competitive | Allosteric | | all. |
| Name | (M) | model | model | appDGD | tive) | steric) | Difference | Difference | Factor | corDGD |
| histamine | 0.0000186 | 0.0000048 | 0.000005 | −26.5 | −29.8 | −29.7 | 3.3 | 3.2 | 0.3 | −26.2 |
| phenylethylamine | 0.0000072 | 0.00000119 | 0.000002 | −28.8 | −33.2 | −32.0 | 4.4 | 3.1 | 0.4 | −28.4 |
| tyramine | 0.000039 | 0.0000075 | 0.000008 | −24.7 | −28.7 | −28.6 | 4.0 | 3.9 | −0.3 | −25.0 |
| dopamine | 0.00001 | 0.0000016 | 0.00000256 | −28.0 | −32.5 | −31.4 | 4.5 | 3.3 | 0.2 | −27.8 |
| tryptamine | 0.000000675 | 0.000000131 | 0.00000019 | −34.6 | −38.6 | −37.7 | 4.0 | 3.1 | 0.5 | −34.2 |
| serotonin | 0.000000138 | 0.000000034 | 0.00000005 | −38.5 | −41.9 | −41.0 | 3.4 | 2.5 | 1.1 | −37.4 |
| norepinehrine 2 | 0.000011 | 0.0000019 | 0.0000019 | −27.8 | −32.1 | −32.1 | 4.3 | 4.3 | −0.7 | −28.5 |
| epinephrine 1 | 0.000039 | 0.0000067 | 0.0000067 | −24.7 | −29.0 | −29.0 | 4.3 | 4.3 | −0.7 | −25.5 |
| melatonin | 0.00002 | 0.00000285 | 0.0000032 | −26.4 | −31.1 | −30.8 | 4.7 | 4.5 | −0.9 | −27.3 |
| MB | 0.000000085 | 0.000000018 | 0.000000026 | −39.7 | −42.4 | −42.5 | 3.8 | 2.9 | 0.7 | −39.0 |
| phenylalanine | 0.000096 | 0.000018 | 0.00002 | −22.5 | −26.6 | −26.4 | 4.1 | 3.8 | −0.3 | −22.8 |
| tyrosine | 0.0000094 | 0.0000014 | 0.0000018 | −28.2 | −32.8 | −32.2 | 4.6 | 4.0 | −0.5 | −28.7 |
| L-DOPA | 0.000016 | 0.0000036 | 0.0000043 | −26.9 | −30.5 | −30.1 | 3.6 | 3.2 | 0.3 | −26.6 |
| tryptophan | 0.0000012 | 0.00000023 | 0.00000035 | −33.2 | −37.2 | −36.2 | 4.0 | 3.0 | 0.5 | −32.7 |
| phenylalanine- | 0.000002 | 0.000000383 | 0.00000039 | −32.0 | −36.0 | −35.9 | 4.0 | 4.0 | −0.4 | −32.4 |
| amid | | | | | | | | | | |
| tyrosine- | 0.000000756 | 0.000000216 | 0.000000242 | −34.3 | −37.4 | −37.1 | 3.1 | 2.8 | 0.8 | −33.6 |
| amid | | | | | | | | | | |
| Phe-1 | 0.000073 | 0.000019 | 0.000021 | −23.2 | −26.5 | −26.2 | 3.3 | 3.0 | 0.5 | −22.7 |
| Phe-2 | 0.000054 | 0.000012 | 0.000013 | −23.9 | −27.6 | −27.4 | 3.7 | 3.5 | 0.1 | −23.9 |
| Phe-3 | 0.00014 | 0.000033 | 0.000036 | −21.6 | −25.1 | −24.9 | 3.5 | 3.3 | 0.2 | −21.4 |
| methylamine | 0.0123 | 0.00169 | 0.0021 | −10.7 | −15.5 | −15.0 | 4.8 | 4.3 | −0.8 | −11.5 |
| methylbutylamine | 0.00033 | 0.000091 | 0.000108 | −19.5 | −22.7 | −22.2 | 3.1 | 2.7 | 0.8 | −18.7 |
| glycine | 0.00305 | 0.00077 | 0.00077 | −14.1 | −17.5 | −17.5 | 3.4 | 3.4 | 0.2 | −13.9 |
| glycine- | 0.000312 | 0.00006 | 0.00007 | −19.7 | −23.7 | −23.3 | 4.0 | 3.6 | −0.1 | −19.8 |
| amide | | | | | | | | | | |
| GABA | 0.036 | 0.011 | 0.013 | −8.1 | −11.0 | −10.6 | 2.9 | 2.5 | 1.1 | −7.0 |
| GABA- | 0.000705 | 0.000152 | 0.00017 | −17.7 | −21.4 | −21.1 | 3.7 | 3.5 | 0.1 | −17.6 |
| amid | | | | | | | | | | |
| glutamine | 0.00077 | 0.000154 | 0.00016 | −17.5 | −21.4 | −21.3 | 3.9 | 3.8 | −0.3 | −17.7 |
| leucine | 0.063 | 0.0094 | 0.0097 | −6.7 | −11.4 | −11.3 | 4.6 | 4.6 | −1.0 | −7.7 |
| agmatine | 0.00000245 | 0.000000214 | 0.000000476 | −31.5 | −37.4 | −35.5 | 5.9 | 4.0 | −0.4 | −31.9 |
| arginine | 0.000089 | 0.0000105 | 0.0000115 | −22.7 | −27.9 | −27.7 | 5.2 | 5.0 | −1.4 | −24.2 |
| ammonia | 0.00132 | 0.000418 | 0.0005 | −16.2 | −19.0 | −18.5 | 2.8 | 2.4 | 1.2 | −15.0 |
| | | | | AVG: | | | 4.0 | 3.5 | 0 | |
| | | | | sd | | | 0.71 | 0.67 | | |

ΔG_{D} is calculated from X_{50%}.
ΔG_{B} is calculated from K_{D} or ^{α}K_{D}, which are calculated from KA and X50%, assuming either competitive or allosteric antagonism and the same binding site on R for both X and A.

Second, we observed correlations between ΔG_{D} values and the numbers of heavy (non-hydrogen) atoms in the target hydrophobic fragments (FIGS. 2A-2B). The molecule with the largest hydrophobic surface, methylene blue (9), yielded the highest affinity of all targets. The correlation between methylamine (3) and two planar aromatic amines in our set, 4 and 8, supports an argument that the applied selection pressure directly optimizes affinity in proportion to hydrophobic surfaces, i.e., is based on the functional groups present, and that we can subtract two ΔG_{D} values to isolate the impact of structural changes. We see indications that functional group-based optimization is general, with methylbutylamine (7), histamine (10), and serotonin (11) being very close to the aromatic amine (3, 4, 8) regression line, although caution should be exercised not to overinterpret these results without further structural information (24).

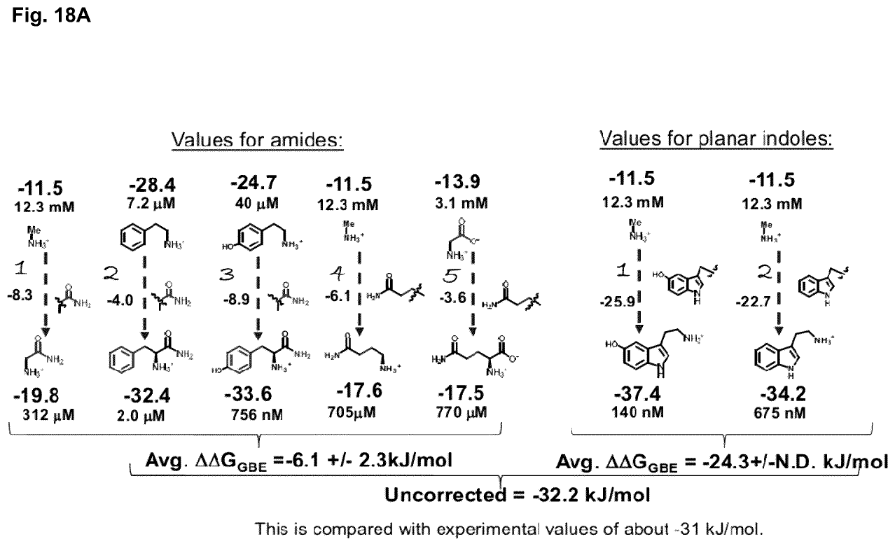

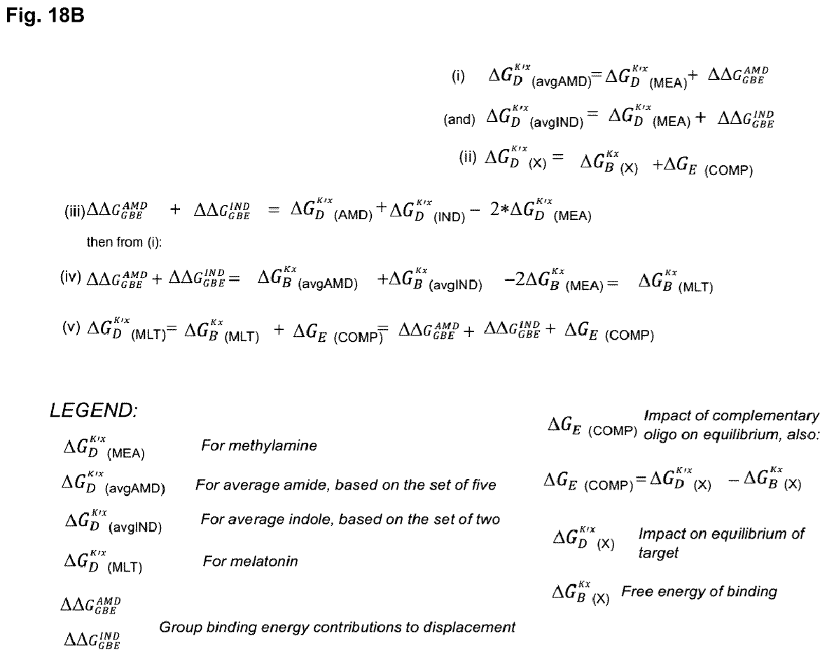

Third, we added average ΔΔG_{GBE} values calculated from two planar indole-containing amines and five primary carboxamides (FIGS. 18A-18B) to obtain a close match with an experimentally determined ΔG_{B} value for melatonin (13), a planar molecule containing an indole and a secondary carboxamide (FIG. 2C, the addition of ΔΔG_{GBE} values leads to ΔG_{B}). Thus, our protocol simultaneously optimizes the presence of multiple functional groups, and we can use this property to interpret deviations from additivity. Our standard selection protocol (FIG. 1B) depends on target-induced oligonucleotide displacement outcompeting background “noise” in the form of more common ligand-independent oligonucleotide release from the column (20, 25). Combinations of target functional groups that reduce the affinity of common aptamers do so by reducing the target occupancy of an aptamer. This decreases the probability of isolating candidate aptamers in the early selection steps, which is critical for selection success.

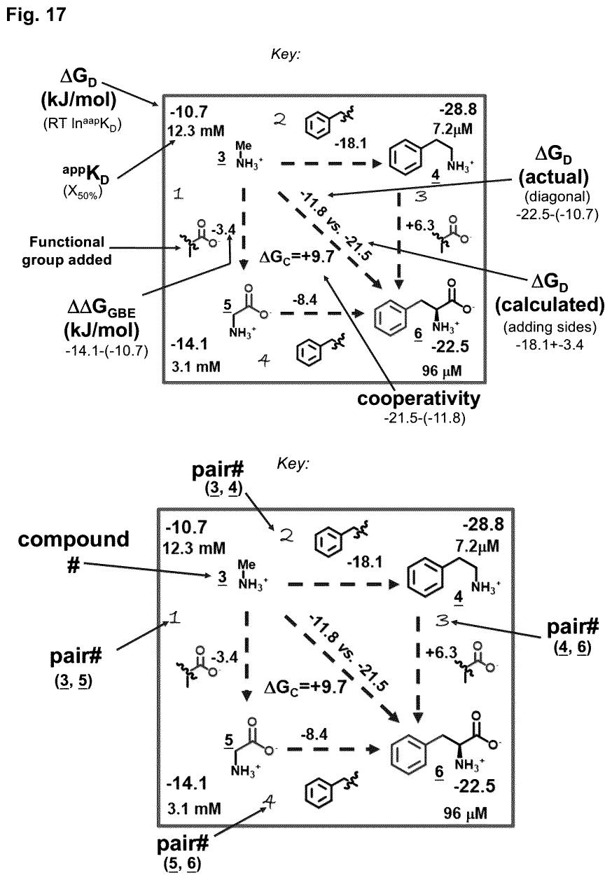

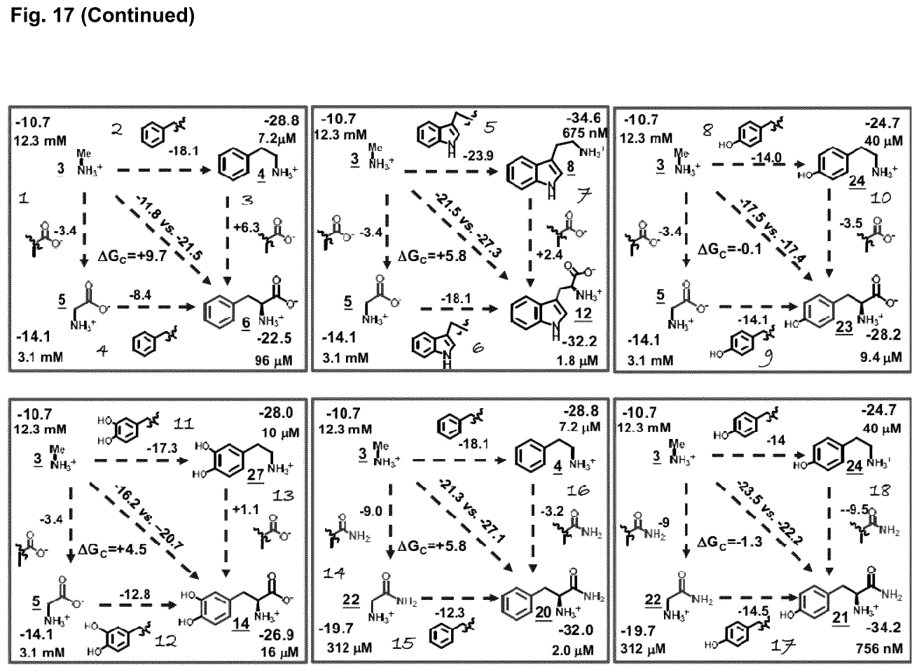

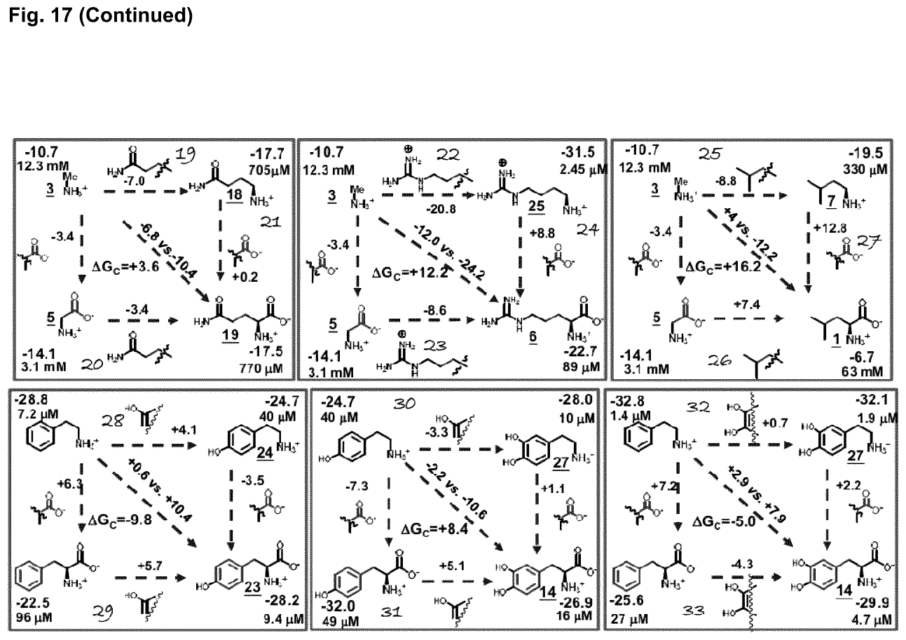

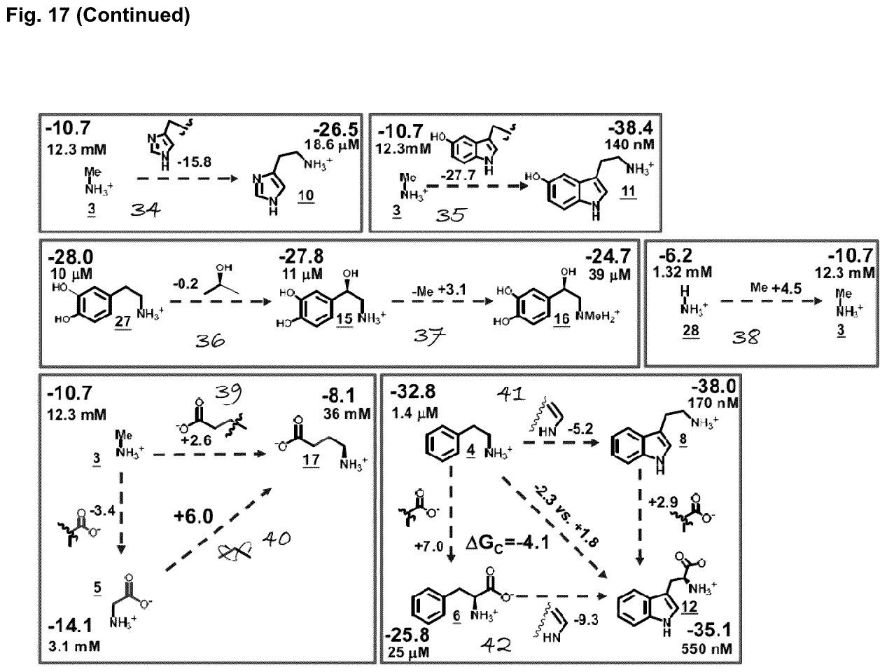

We used the aromatic amine (3, 4, 8) regression line (FIG. 2B) to estimate the impact of additional carboxylates on the ΔG_{D} values for the aptamer-target complexes of two related aromatic amino acids, phenylalanine (6) and tryptophan (12), observing that the addition of a carboxyl group is similar to the loss of receptor hydrophobic contacts for between one and two heavy atoms, which is intuitively consistent with the introduction of a polar carboxylate near a primarily hydrophobic pocket. Further, our analysis of double functional group replacement cycles (FIGS. 1E, 2E, and 17) revealed substantial negative cooperativity while adding negative charge in proximity to mismatched groups, such as hydrophobic residues (in phenylalanine and tryptophan). These structural constellations, then, were identified as likely to reduce the probability of aptamer isolation for leucine.

### Example 4

### Functional Group-Guided Selections for Leucine

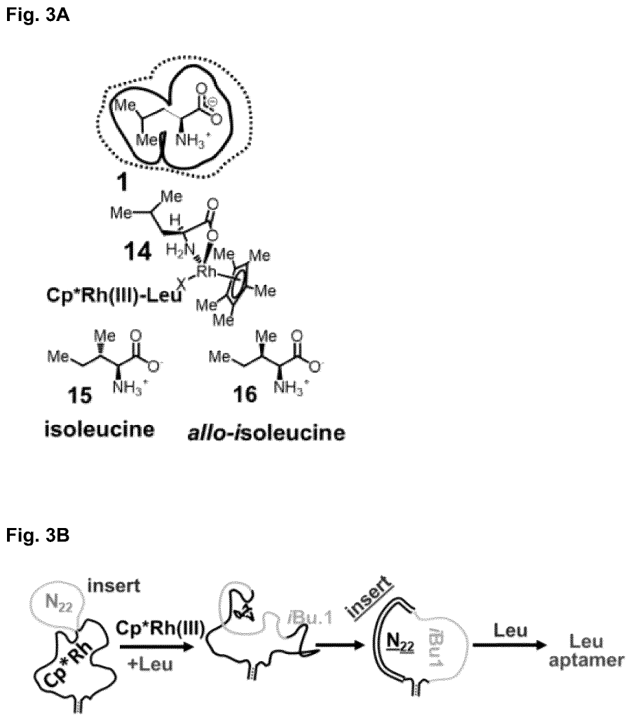

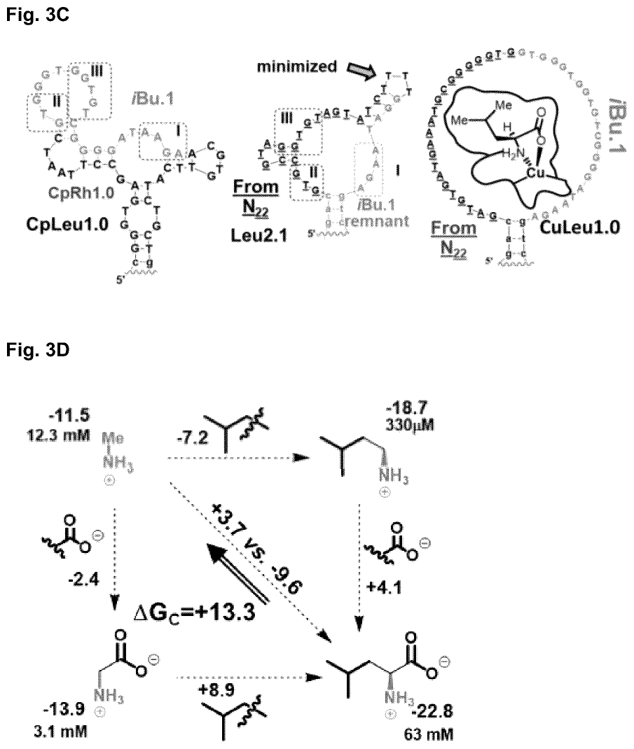

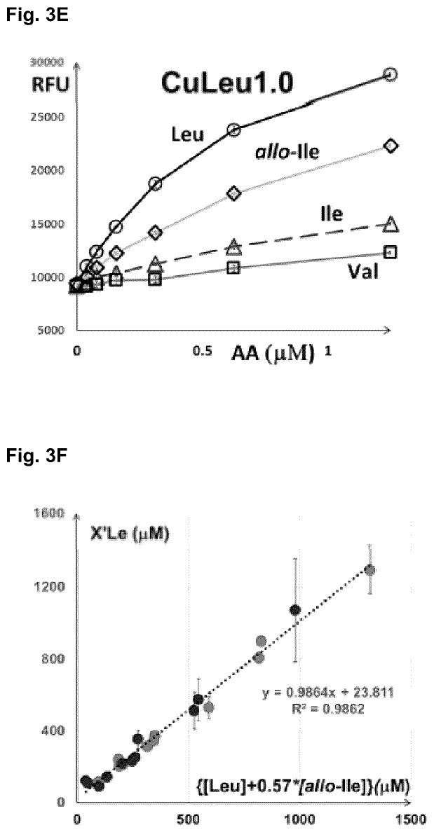

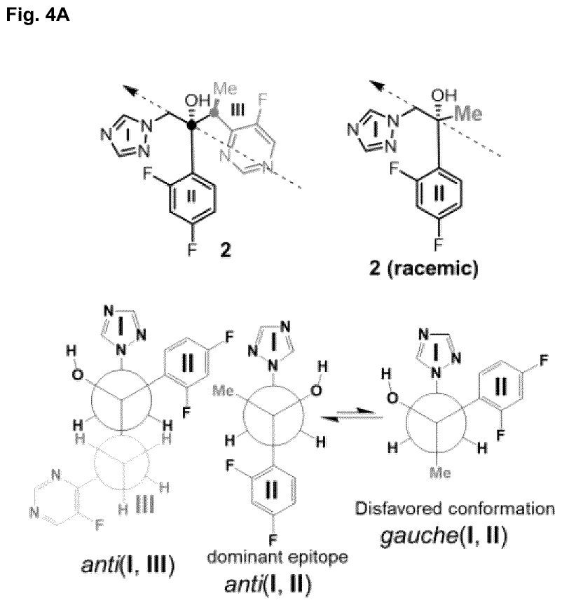

We extended our analysis to hypothesize that the two out-of-plane carbons and a carboxylate, all in proximity in leucine, act synergistically to minimize contact surfaces and reduce affinities of typical aptamers, thus allowing competing ligand-independent release mechanisms to dominate and suppress the desired outcomes. To overcome this issue, we separated the selection steps for the alkyl (isobutyryl) and α-amino-carboxylate groups (FIGS. 3A-3B). We first implemented a protocol to identify a sequence iBu.1 certain to contain a binding motif for the isobutyl group. We started with a Cp*Rh(III)-binding aptamer (CpRh1.0), specifically isolated for this purpose, as a temporary placeholder for sequences that interact with the carboxyl and amino groups (26). We inserted a random 22-mer region which will become iBu, into the CpRh1.0, creating a new library (n.b., we can screen complete 22-mer sequence space). From this library, we selected aptamers like CpLeu1.0, which bound leucine in the presence of the Cp*Rh(III) cofactor. While we could immediately eliminate CpLeu1.0 from further consideration as a Leu sensor, because of its complex mechanism of interactions with leucine, reflected in a sharp threshold behavior of fluorescent sensor (cf. FIG. 16C), we knew that the inserted sequence, iBu, had to contain binding motifs for the Leu side chain.

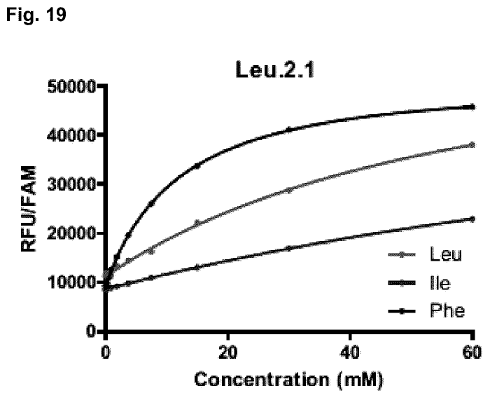

We then designed a library of 22-mers with iBu.1 positioned next to the stem. From this library, using elutions with leucine sans cofactor, we identified Leu2.1 (FIG. 3C). The Leu2.1 aptamer had a K_{D} of almost 10 mM (FIG. 16C) and an ˜4:1 preference for Leu over lie (FIG. 19). The negative cooperativity (ΔG_{C}) between the carboxyl and isobutyryl groups was large (>10 kJ/mol), providing an explanation for our initial selection difficulties (FIG. 3D).

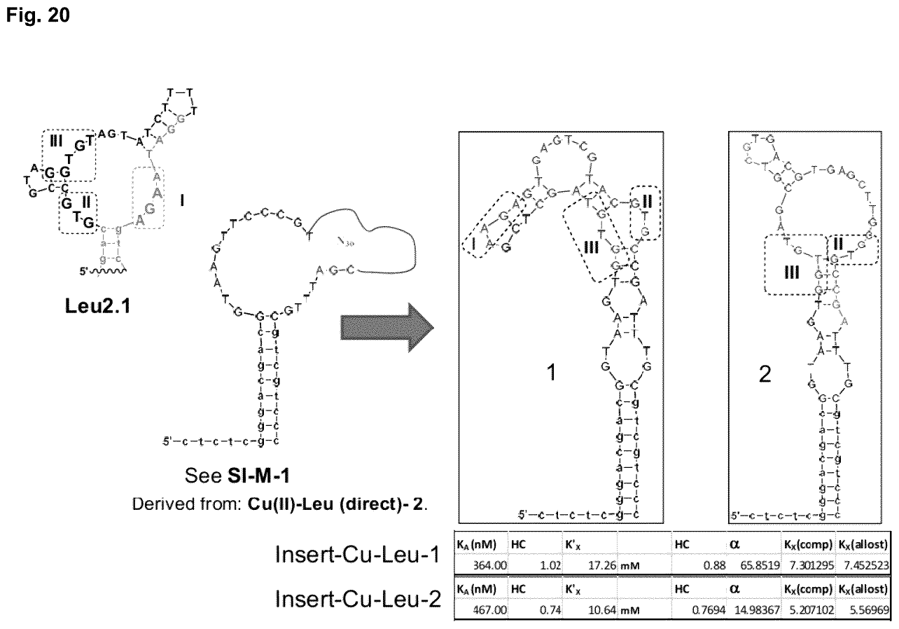

We identified homologous regions 1-III in CpLeu1.0 and Leu2.1, two of which, II and III, in Leu2.1 originated from the inserted random region outside of iBu.1. The short Leu2.1 aptamer should have been abundant in any initial pool, further, isolation of motifs II and III in control studies of insertion reselection (FIG. 20), indicated that the motif I is not absolutely required in Leu aptamers. However, apparently due to its low affinity, Leu2.1 required the prefixed compatible sequences within I, to increase the probability of isolation via a reduction in the required sequence length in the newly inserted random region.

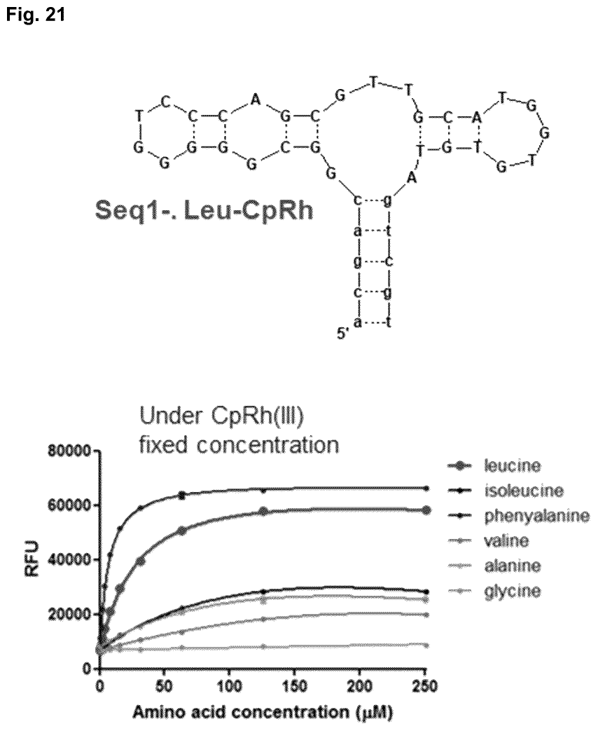

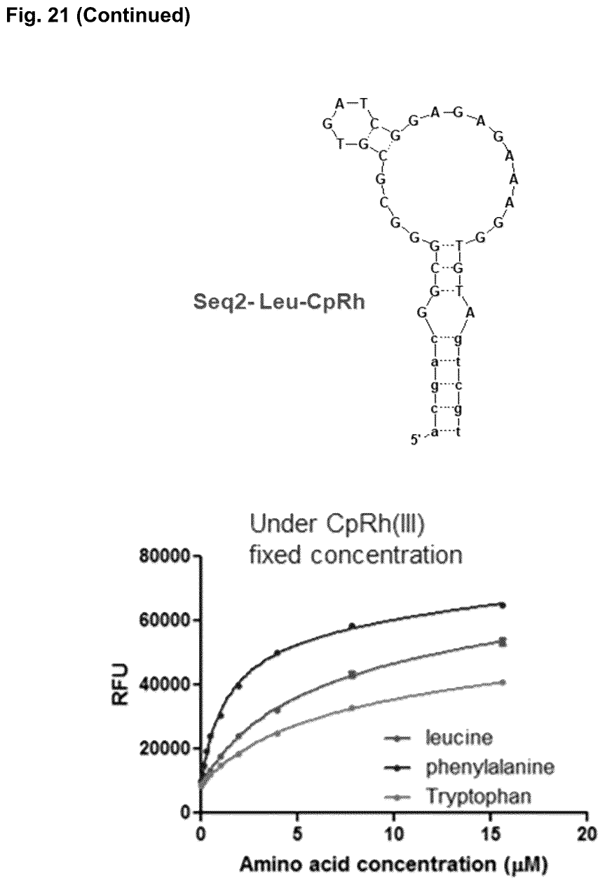

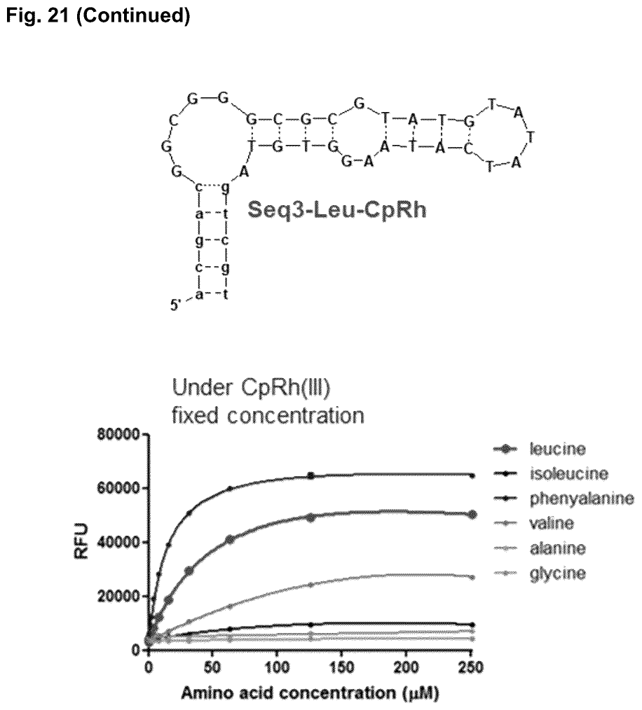

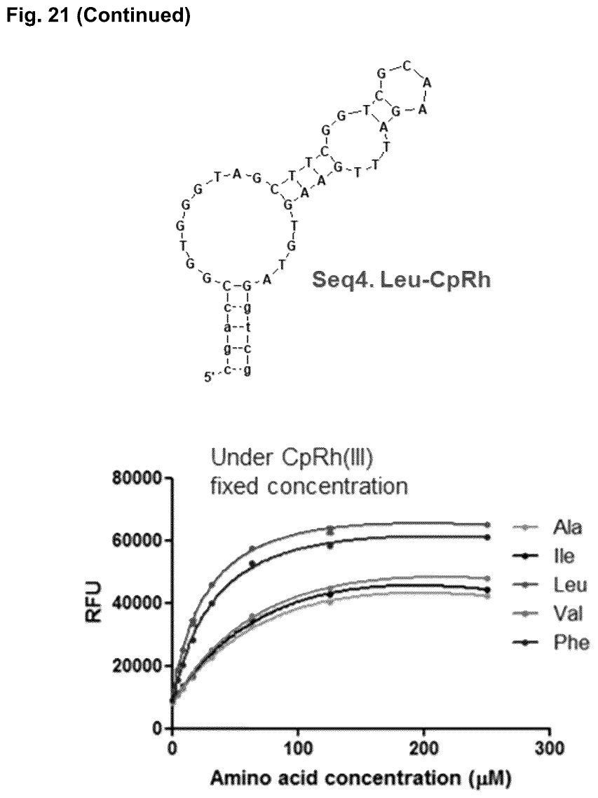

The Leu2.1 aptamer had a millimolar target affinity insufficient for the intended application of testing newborns with MSUD (11). Further, Leu2.1 preferred phenylalanine over leucine (FIG. 19), which was a dominant problem in our prior selections using Cp*Rh(III) as the cofactor (FIG. 21 and Table 5).

TABLE 5
Sequences tested in direct selection for Leu aptamers
using Cp*Rh(III) cofactor.

| No. ID | Leu-CpRh, Sequence composition (5′-3′) |
|---|---|
| N30 lib. | GGA GGC TCT CGG GAC GAC N(30)GTC GTC CCG ATG CTG CAA |
| | TCG TAA (SEQ ID NO: 31) |
| 1 | ACG ACG GCG GGG GTC CCA GCG TTG CAT GGT GTG TAG TCG T |
| | (SEQ ID NO: 32) |
| 2 | ACG ACG GOG GGC GCG TGA TCG GAG AGA AAG GTG TAG TCG T |
| | (SEQ ID NO: 33) |
| 3 | ACG ACG GCG GGC GCG TAT GTA TAT CAT AAG GTG TAG TCG T |
| | (SEQ ID NO: 34) |
| N36 lib | Same library used for other GBE aptamer isolation, |
| | with Phe/Ile counterselection |
| 4 | -CG ACC GGT GGG TAG CTT CGG TCG CAA GAT TTG AAG TGT |
| | AGG TCG (SEQ ID NO: 35) |

Thus, we added an aminophilic cofactor, Cu(II) (27), in the last step of the selection to improve affinity and selectivity. We hypothesized that Cu(II) would serve as a protecting group neutralizing the effects of the carboxylate through the complexation with the 2-amino-ethanoate group. Complexation would allow better access of hydrophobic DNA monomer residues to the leucine side chain, improving affinity and selectivity. Consistent with our hypothesis, we identified aptamer CuLeu1.0 having a 44-mer loop, conserved sections of iBu.1, and high affinity for leucine (Ko-170 nM; FIG. 3C).

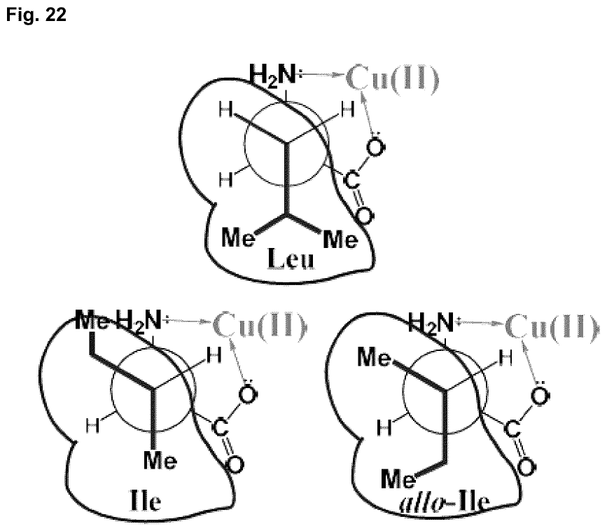

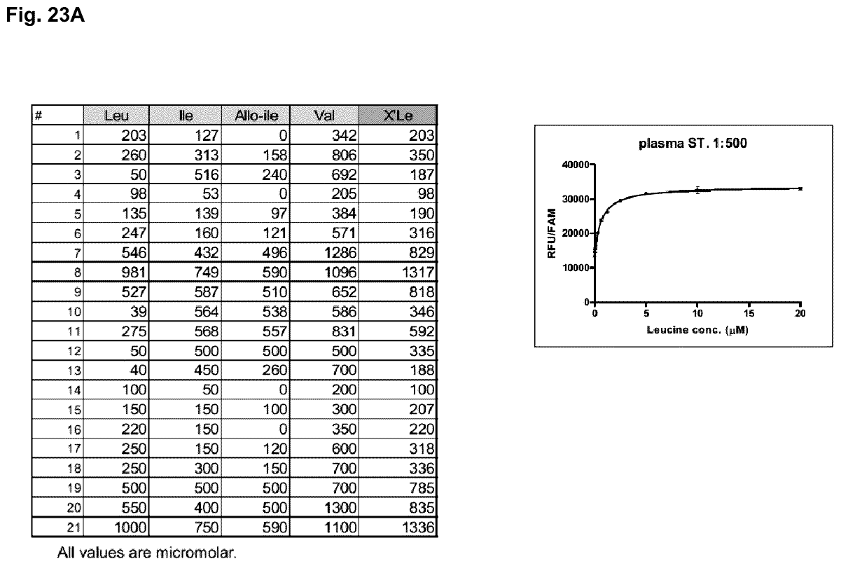

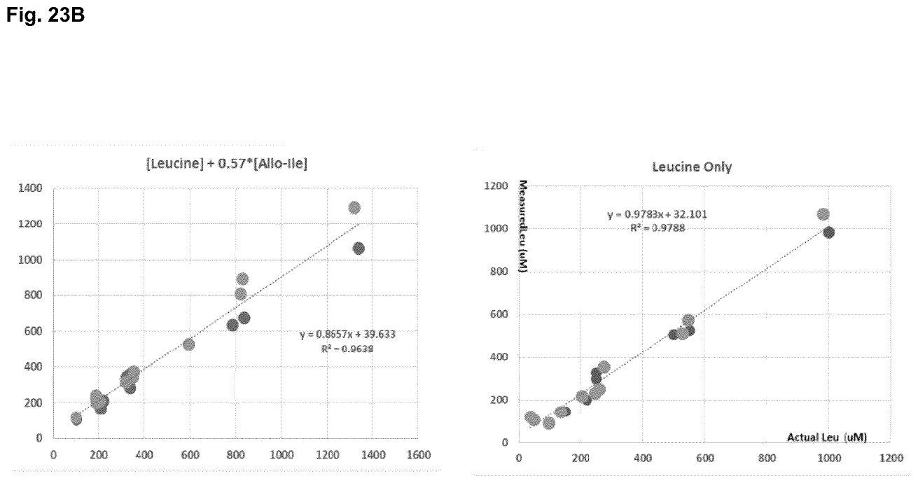

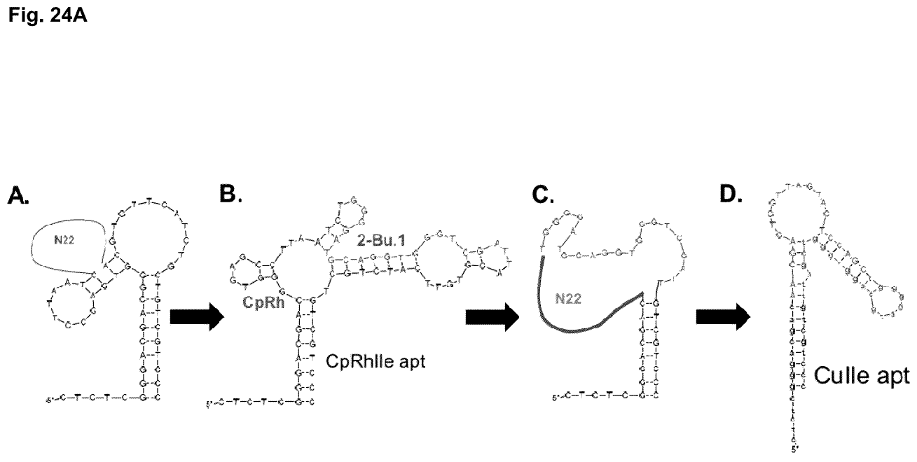

While CuLeu1.0 had selectivity for leucine over isoleucine, valine, and phenylalanine, we noted strong cross-reactivity with allo-isoleucine (FIG. 3E, Newman projections in FIG. 22). The allo-isoleucine metabolite was not used in the aptamer counter-selections because its concentrations are negligible at birth. Currently, newborn screening is performed with mass spectroscopy (11), which integrates isobaric species to provide XLe values (XLe=Leu+Ile+allo-IIle+2OHPro). Thus, our aptamer sensor is a candidate for the development of rapid tests to address false positives in MSUD by showing a lack of steady increase in X′Le values in consecutive measurements, with X′Le defined, e.g., as [Leu]+0.57*[allo-IIle] (FIG. 3F and FIGS. 23A-23B). After the first few days of life, however, allo-Ile concentrations increase so a monitoring strategy without fully specific aptamers would require a cross-reactive array (26, 28), for which CuLeu1.0 is a suitable component.

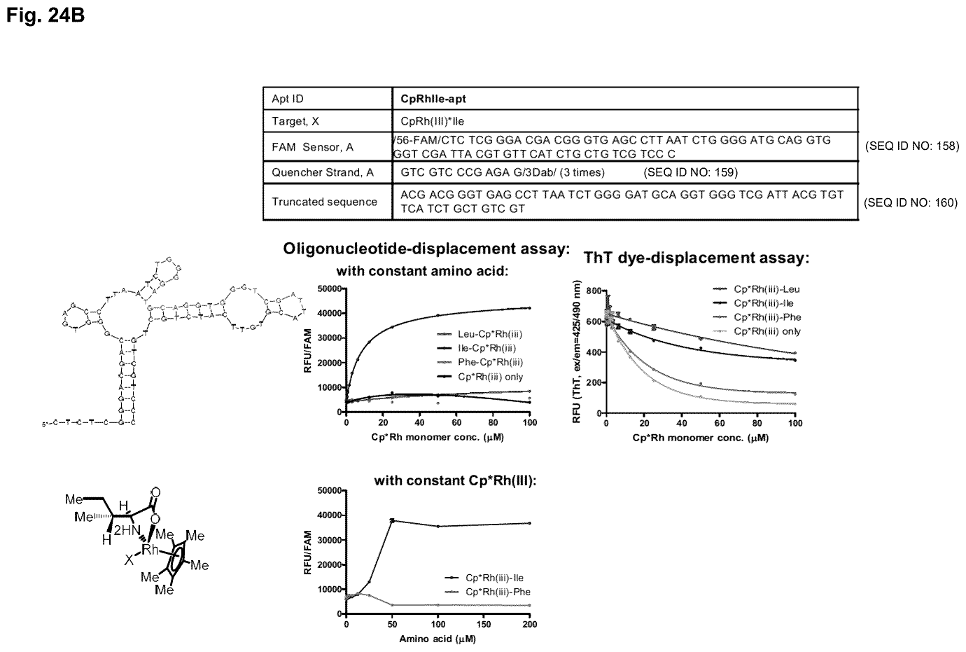

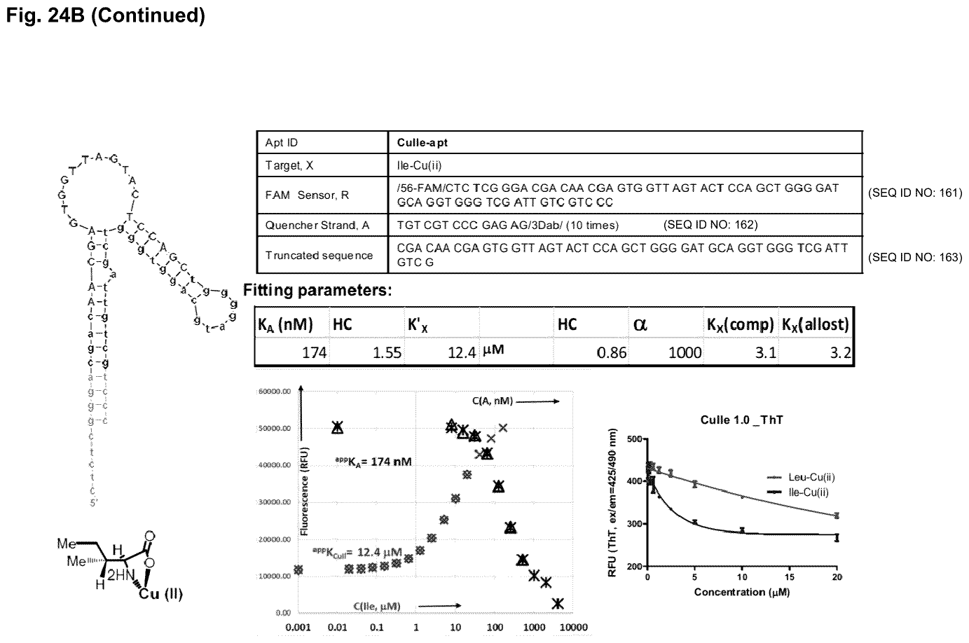

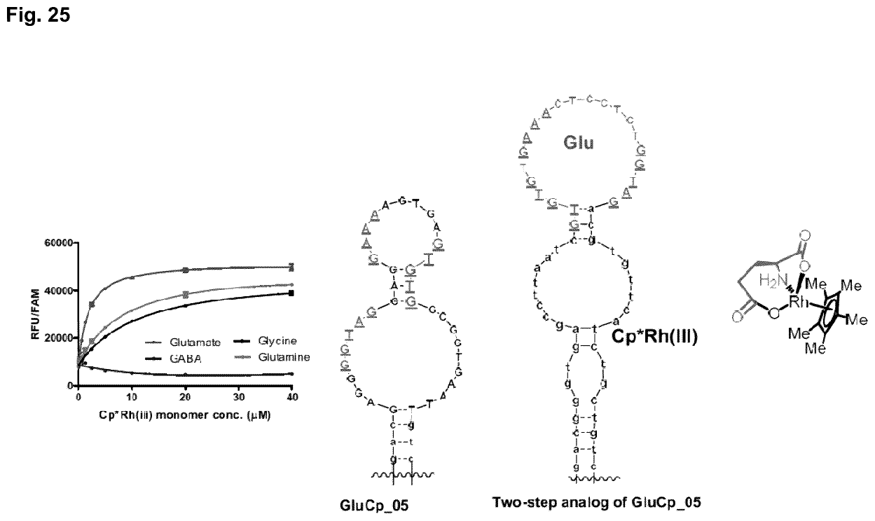

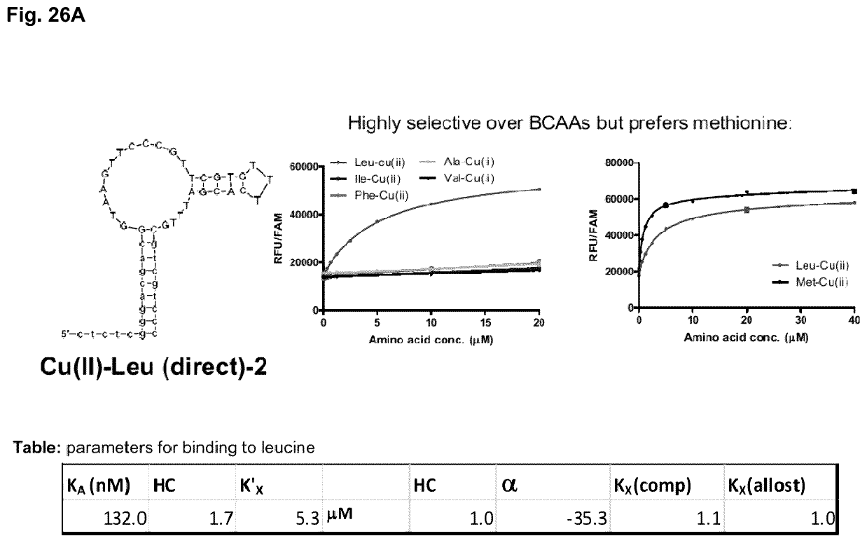

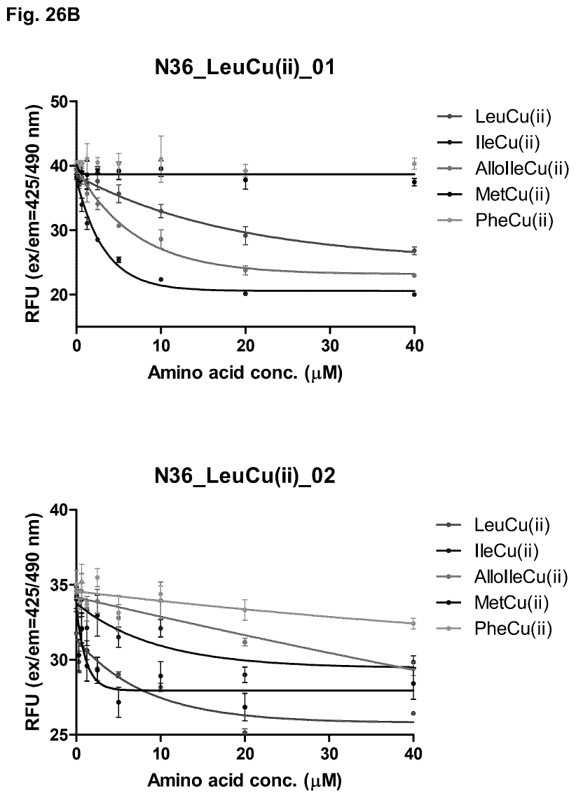

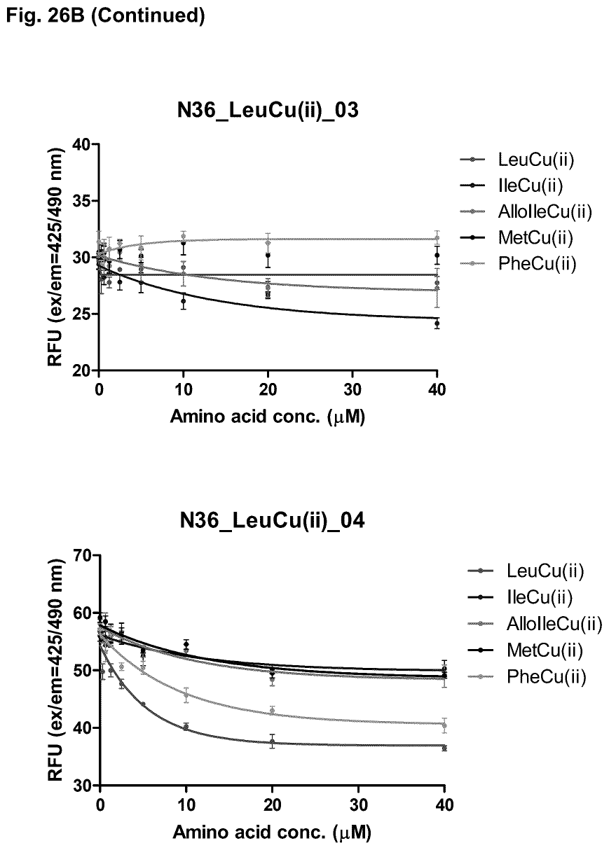

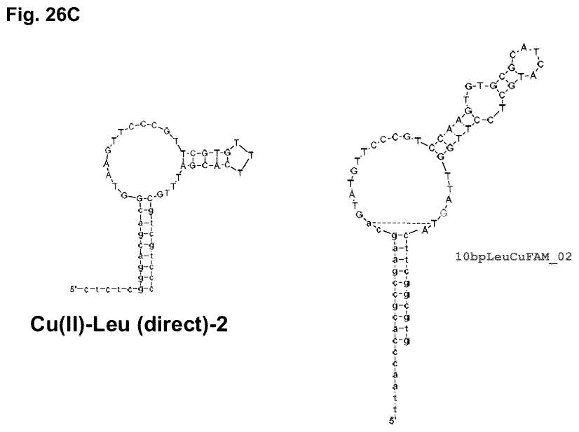

The multi-step approach with Cu(II) can be generalized to amino acids that display a side chain away from the Cp*Rh(III) complex, such as Ile (cf. CuIle1.1, FIGS. 24A-24B). This approach would not work for amino acids that carry a chelating group beyond 2-aminoethanoate, e.g., glutamate (FIG. 25). For comparison, we performed a single-step Leu selection with Cu(T) as the cofactor. We isolated receptors with about fivefold lower affinities compared to CuLeu1.0. The two most abundant sequences preferred isoleucine or methionine (FIGS. 26A-26C, Table 6). These aptamers are candidates for arrays.

TABLE 6
The pool of promising hits that were tested. The initial
selection cut-off is above 1%.

| | ctctcgggacgac_nnnnnnnnnnnnnnnnnnnn | |
|---|---|---|
| | nnnnnnnnnnnnnnnn_gtcgtccc (SEQ ID | % in |
| Lib | NO: 36) | total |
| N36_LeuCu(ii)_01 | ctctcgggacgac_CGTTTGAACTCAATTAGGTT | 29.50 |
| | GCATCGGTCCCACGAG_gtcgtccc (SEQ ID | |
| | NO: 37) | |
| N36_LeuCu(ii)_02 | ctctcgggacgac_GGTAAGTTCCCGTTCATGTT | 29.47 |
| | GTCCATCATGATTTGC_gtcgtccc (SEQ ID | |
| | NO: 38) | |
| N36_LeuCu(ii)_03 | ctctcgggacgac_CAGTTTGGCGTTTTGTACGC | 10.93 |
| | GCATCGGTCCCACGTG_gtcgtccc (SEQ ID | |
| | NO: 39) | |
| N36_LeuCu(ii)_04 | ctctcgggacgac_GGCCCCGGGGCAAACAACAG | 9.77 |
| | CATAGGTTGTTTACGA_gtcgtccc (SEQ ID | |
| | NO: 40) | |
| N36_LeuCu(ii)_05 | ctctcgggacgac_GGGCAATTTGTAGCCTGATA | 3.25 |
| | GTAAGTTCCCGTTGAAC_gtcgtccc (SEQ ID | |
| | NO: 41) | |
| N36_LeuCu(ii)_06 | ctctcgggacgac_GGGTAAGTTCCCGTACGCTA | 3.04 |
| | TACATAACGTTTTGTC_gtcgtccc (SEQ ID | |
| | NO: 42) | |
| N36_LeuCu(ii)_07 | ctctcgggacgac_GAGAAAAGGGTTGATCAATA | 1.45 |
| | CTATTGGAATGTCGCA_gtcgtccc (SEQ ID | |
| | NO: 43) | |
| N36_LeuCu(ii)_08 | ctctcgggacgac_GGCCAATGGGAGAAATTAGG | 1.05 |
| | GGTAGCCGCCTGGGTT_gtcgtccc (SEQ ID | |
| | NO: 44) | |
| N36_LeuCu(ii)_09 | ctctcgggacgac_CGCCTGACTACAAGTGGTAG | 1.05 |
| | ACATGGGAGCAATTAG_gtcgtccc (SEQ ID | |
| | NO: 45) | |
| N36_LeuCu(ii)_10 | ctctcgggacgac_GGTTCCAGGGCACCAAGATG | 0.80 |
| | ATTAATCGTGGTACGA_gtcgtccc (SEQ ID | |
| | NO: 46) | |
| N36_LeuCu(ii)_11 | ctctcgggacgac_CAGTAAGTTCCCGTACGCAT | 0.50 |
| | TTGGTTAGTACTTGTG_gtcgtccc (SEQ ID | |
| | NO: 47) | |
| N36_LeuCu(ii)_12 | ctctcgggacgac_GGAGTGTGGGAGAAATTAGC | 0.34 |
| | ATACAGCGCCCAGGAT_gtcgtccc (SEQ ID | |
| | NO: 48) | |
| N36_LeuCu(ii)_13 | ctctcgggacgac_CATTTGTCCTGTGAACATAG | 0.15 |
| | GAGTAAGTGCCCGTTG_gtcgtccc (SEQ ID | |
| | NO: 49) | |

### Example 5

### A Structure-Guided Approach to Aptamers for Voriconazole

Leucine (1) is closely related to other amino acids in our target set. By contrast, voriconazole (2) is an example of applying a structurally guided approach to unrelated molecules. We initially attributed our voriconazole selection failures to its limited solubility (˜200 μM). Nonetheless, selections using a soluble voriconazole phosphate analog also failed. Similar to leucine, we considered that voriconazole has a sterically crowded environment (FIG. 4A) forcing its fragments (structural subunits) into a propeller-like conformation, as revealed in crystal structures (30). This sterically crowded conformation was hypothesized to lead to suboptimal access to hydrophobic surfaces in DNA needed to interact with fragments I-III (FIG. 4A). One possible retrosynthetic disconnection (31) led to a simplified, less congested, and readily synthesized alcohol analog, 2a (FIG. 4A).

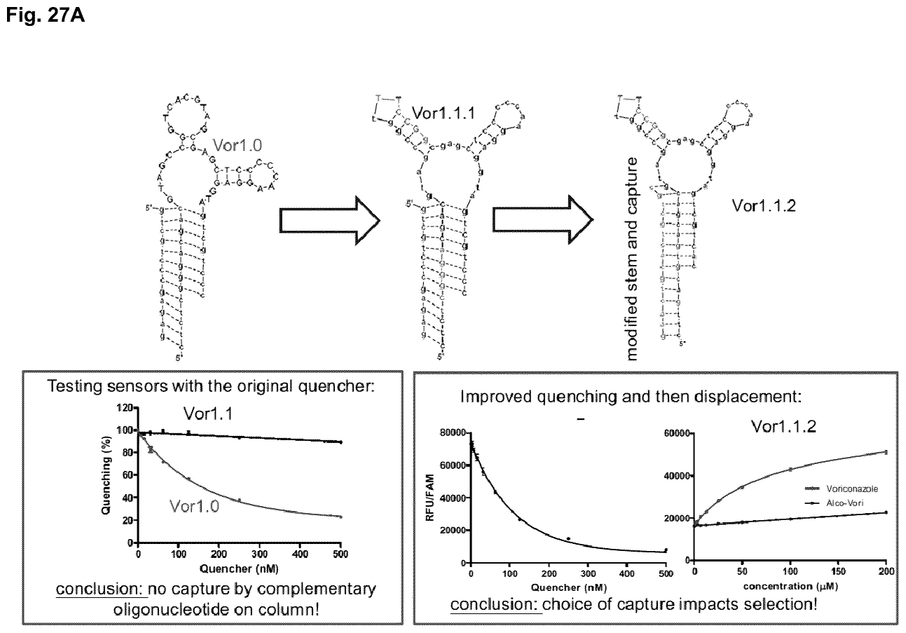

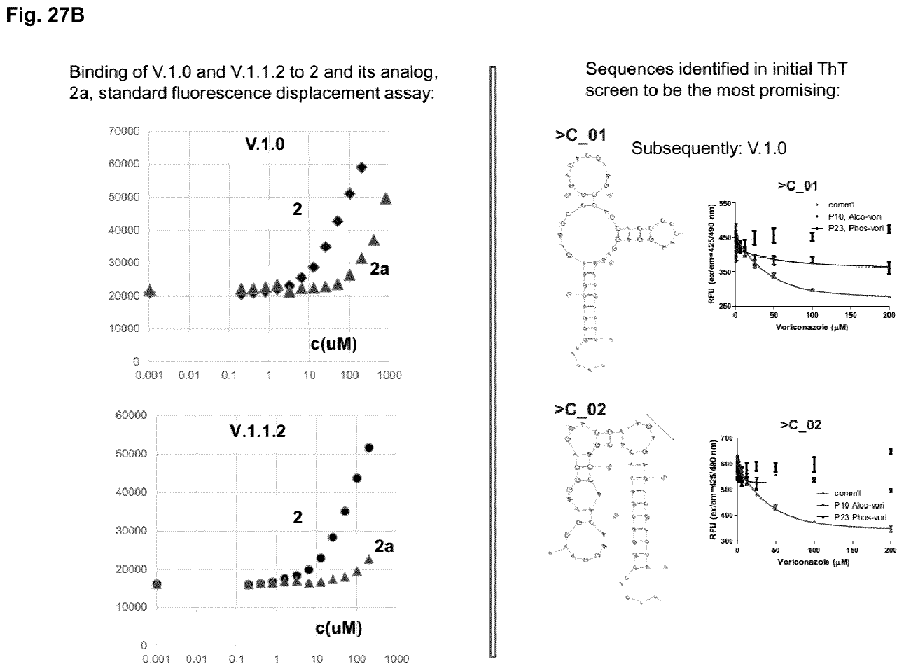

Initial attempts starting with 2a at high concentrations, while introducing voriconazole separately in later cycles, failed, yielding exclusively analog-binding aptamers. Further conformational analysis using Newman projections clarified that 2a likely represents a dominant epitope during selection in which fragments I and II are positioned anti. Conversely, in voriconazole, structural subunits are gauche (FIG. 4A). Inspired by approaches to outflank the immunodominance of epitopes (32), we mixed 2 and 2a at their respective maximal concentrations in the initial selection steps, only gradually phasing out the analog. We hypothesized that this procedure would maximize the probability of release of aptamers that bind similar conformations of the target and its analog, which could be important in the initial rounds of selection. In contrast to previous failures, this change led to two aptamers (FIGS. 27A-27B) responsive to 2 and 2a (FIGS. 4B-4C), confirming the advantage of adding the analog.

The mechanisms underlying the improved selection strategy for voriconazole are partially unclear because we cannot exclude the possibility that the analog minimizes target aggregation. Nonetheless, the presence of 2a is certain to improve target-receptor occupancy in the initial cycles, likely buttressing low effective concentrations of monomeric voriconazole in conformations that can elicit aptamers. The isolated aptamers do not bind fluconazole, suggesting they are not class-wide cross-reactive aptamers (28, 29, 17) and that stabilizing interactions occur with group III in 2 (FIG. 4A).

Mutagenesis studies indicated that our lead aptamer, Vor1.0, is a destabilized three-way junction (FIGS. 4C-4D), which we turned into a FRET sensor Vor1.1.4 (FIG. 4D). The latter shows sufficient sensitivity for testing as an electrochemical sensor for in vivo use (12). This specific family of voriconazole-binding three-way junctions, despite being common, are eliminated from direct selections by exceptionally poor interactions with capture oligonucleotides, which was prevented in Vor1.0 by structure switching (FIG. 4C and FIGS. 27A-27B). These observations showcase the complex balance between positive and negative selection pressures in our protocols. Our new procedures, as demonstrated through leucine and voriconazole selections, shift selection balance in our favor by addressing probabilistic barriers assigned to crowded (and other non-optimal) substructures within targets.

### Example 6

### Conclusions

In traditional organic synthesis, the cartoonish concept of functional groups and their reactivities guides us through transformations involving relationships between nuclei and electron clouds (33). Here, in structure-guided aptamer selections, analogous concepts directed random searches through the space of complementary interactions between targets and aptamer receptors. We developed several approaches that can be used in functional-group guided selections. These include insertion reselection, carrying-over and anchoring of partial motifs, the expanded use of metal complexes as “protective” groups, placeholders, cross-linkers, and the synthesis of simpler analogs designed to overcome steric or conformational barriers. These approaches can be further studied, optimized, and combined with one another and with traditional protocols (3, 4), organic receptor cofactors (6, 34), and modified bases (35), while considering library designs (25), to enable isolation of high-quality aptamers and engineering of biosensors for previously inaccessible targets.

We have steered away from three topics for which our approach may provide information for further consideration. First, there is the question of natural selection of complex functions in the hypothetical, pre-protein, RNA world (36). Behaving as tinkerers (37), we reused simple sequence pieces to find new functions, building on the early work on the use of cofactors in RNA catalysis (38). Second, our thinking about uncovering selection barriers for optimal receptors could be inverted to provide insights into finding small-molecule drugs that specifically modulate natural nucleic acid targets (39). And third, we provide a wide set of sequences with confirmed target binding that could be used to improve training sets for computational designs (40).

### DOCUMENTS CITED

1. A. D. Ellington, J. W. Szostak, In vitro selection of RNA molecules that bind specific ligands. Nature 346, 818-822 (1990).
2. C. Tuerk, L. Gold, Systematic evolution of ligands by exponential enrichment: RNA ligands to bacteriophage T4 DNA polymerase. Science 249, 505-510 (1990).
3. A. Ruscito, M. C. DeRosa, Small-molecule binding aptamers: Selection strategies, characterization, and applications. Front. Chem. 4, 1-14 (2016).
4. H. Yu, O. Alkhamis, J. Canoura, Y. Liu, Y. Xiao, Advances and challenges in small-molecule DNA aptamer isolation, characterization, and sensor development. Angew. Chem. Int. Ed. 60, 16800-16823 (2021).
5. N. Nakatsuko et al., Aptamer-field-effect transistors overcome Debye length limitations for small molecule sensing. Science 362, 319-324 (2018).
6. K. A. Yang et al., Recognition and sensing of low-epitope targets via ternary complexes with oligonucleotides and synthetic receptors. Nat. Chem. 6, 1003-1008 (2014).
7. K. M. Cheung, et al., Phenylalanine monitoring via aptamer-field-effect transistor sensors ACS Sensors, 4, 3308-3317 (2019).
8. N. Tejavibulya, et al., Hydrogel microfilaments toward intradermal health monitoring. iScience, 21, 328-340 (2019).
9. B. S. Ferguson, et al., Real-time, aptamer-based tracking of circulating therapeutic agents in living animals. Sci. Transl. Med. 5, 213ra165 (2013).
10. C. Zhao et al., Implantable aptamer-field-effect transistor neuroprobes for in vivo neurotransmitter monitoring. Science Advances 7:eabj7422 (2022).
11. K. Stroek, et al., Evaluation of 11 years of newborn screening for maple syrup urine disease in the Netherlands and a systematic review of the literature: Strategies for optimization. JIMDreports 54, 68-78 (2020).
12. P. Dauphin-Ducharme et al., Electrochemical aptamer-based sensors for improved therapeutic drug monitoring and high-precision, feedback-controlled drug delivery. ACS Sens. 4, 2832-2837 (2019).
13. H. Elewa, E. El-Mekaty, A. El-Bardissy, M. H. H. Ensom, K. J. Wilby, Therapeutic drug monitoring of voriconazole in the management of invasive fungal infections: A critical review, Clin. Pharmacokinet. 54, 1223-1235 (2015).
14. R. Nutiu, Y. Li, In vitro selection of structure-switching signaling aptamers. Angew. Chem. Int. Ed. 44, 1061-1065 (2005).
15. M. Rajendran, A. D. Ellington, Selection of fluorescent aptamer beacons that light up in the presence of zinc. Anal. Bioanal. Chem. 390, 1067-1075 (2008).
16. M. Yarus, Amino acids as RNA ligands: A direct-RNA-template theory for the code's origin. J. Mol. Evol. 47, 109-117 (1998).
17. G. R. Wiedman, Y. Zhao, D. S. Perlin, A Novel, Rapid, and low-volume assay for therapeutic drug monitoring of posaconazole and other long-chain azole-class antifungal drugs. mSphere, 3, ee00623-18 (2018).
18. F. J. Ehlert, Estimation of the affinities of allosteric ligands using radioligand binding and pharmacological null methods.” Mol. Pharm. 33, 187-194 (1987).
19. S. M. Free, J. W. Wilson, A mathematical contribution to structure-activity studies. J. Med. Chem. 7, 395-399 (1964).
20. C. Bissantz, B. Kuhn, M. Stahl, A medicinal chemist's guide to molecular interactions. J. Med. Chem. 53, 5061-5084 (2010).
21. J. R. Lorsch, J. W. Szostak, Chance and necessity in the selection of nucleic acid catalysts. Acc. Chem. Res. 29, 103-110 (1996).
22. S. E. Osborne, A. D. Ellington, Nucleic acid selection and the challenge of combinatorial chemistry. Chem. Rev. 97, 349-370 (1997).
23. H. Gohlke, H. Klebe, Approaches to the description and prediction of the binding affinity of small-molecule ligands to macromolecular receptors. Angew. Chem. Int. Ed. 41, 2644-2676 (2002).
24. B. Baum et al., Non-additivity of functional group contributions in protein-ligand binding: A comprehensive study by crystallography and isothermal titration calorimetry. J. Mol. Biol. 397, 1042-1054 (2010).
25. J. M. Carothers, S. C. Oestreich, J. H. Davis, J. W. Szostak, Informational complexity and functional activity of RNA structures. J. Am. Chem. Soc. 126, 5130-5137 (2004).
26. A. Buryak, K. Severin, A chemosensor array for the colorimetric identification of 20 natural amino acids. J. Am. Chem. Soc. 127, 3700-3701 (2005).
27. Z. Liu et al., Tuning the reactivity of nitriles using Cu(II) catalysis—potentially prebiotic activation of nucleotides. Chem. Sci. 35, 7052-7057 (2018).
28. K.-A. Yang, R. Pei, D. Stefanovic, M. Stojanović, Optimizing cross-reactivity with evolutionary search for sensors. J. Am. Chem. Soc. 134, 1642-1647, (2012).
29. W. Yang, et al., In vitro isolation of class-specific oligonucleotide-based small-molecule receptors. Nucleic Acids Res. 47, e71 (2019).
30. K. Ravikumar, B. Sridhar, K. D. Prasad, Ak. K. S. Bujanga Rao, Voriconoazole, an antifungal drug. Acta Cryst. E63, 0565-0567 (2007).
31. E. J. Corey, The Logic of Chemical Synthesis. John Wiley & Sons (1989).
32. D. Angeletti, I. Kosic, J. J. S. Santos, J. W. Yewdell, Outflanking immunodominance to target subdominant broadly neutralizing epitopes. PNAS, 116, 13474-13479 (2019).
33. H. Ochiai, The logical structure of organic chemistry and the empirical adequacy of the classical concept of the molecule. HYLE-Int. J. Phil. Chem. 19, 139-160 (2013).
34. J. C. Manimala, S. L. Wiskur, A. D. Ellington, E. V. Anslyn, Tuning the specificity of a synthetic receptor using a selected nucleic acid receptor. J. Am. Chem. Soc. 126, 16515-16519 (2004).
35. F. Pfeiffer et al., Identification and characterization of nucleobase-modified aptamers by click-Selex. Nat. Protoc. 13, 1153-1180 (2018).
36. H. Saito, The RNA world “hypothesis”. Nature Rev. Mol. Cell. Bio. 23, 582 (2022).
37. F. Jacob, Evolution and tinkering. Science 196, 1161-1166 (1977).
38. J. R. Lorsch, J. W. Szostak, In vitro evolution of new ribozymes with polynucleotide kinase activity. Nature, 371, 31-36 (1994).
39. D. K. Warner, C. E. Hajdin, K. M. Weeks, Principles for targeting RNA with drug-like small molecules. Nat. Rev. Drug Discov. 17, 547-558 (2018).
40. W. M. Billings, B. Hedelius, T. Millecam, D. Wingate, D. D. Corte, ProSPr: Democratized implementation of Alphafold protein distance prediction network. bioRxiv doi:10.1101/830273.
41. K.-A. Yang, R. Pei, M. N. Stojanović, In vitro selection and amplification protocols for isolation of aptameric sensors for small molecules. Methods, 106, 58-65 (2016).
42. M. Zuker, Mfold web server for nucleic acid folding and hybridization prediction. Nucleic Acids Res. 31, 3406-3415 (2003).
43. N. Nakatsuka, J. M. Abendroth, K.-A. Yang, A. M. Andrews, Divalent cation dependence enhances dopamine aptamer biosensing, ACS Appl. Mater. Interfaces, 13, 9425-9435 (2021).
44. F. J. Ehlert, Estimation of the affinities of allosteric ligands using radioligand binding and pharmacological null methods. Mol. Pharmacol. 33, 187-194 (1987).
45. T. S. Najdi, C.-R. Yang, B. E. Shapiro, G. W. Hatfield, E. D. Mjolsness, Application of a generalized MWC model for the mathematical simulation of metabolic pathways regulated by allosteric enzymes. J. Bioinform. Comp. Bio. 4, 335-55 (2006).
46. E. F. Douglas, C. J. Miller, G. Sparer, H. Shapiro, D. A. Spiegel, A Comprehensive mathematical model for three-body binding equlibria. J. Am. Chem. Soc. 135, 6092-6097 (2013).
47. A. Brown, Analysis of cooperativity by isothermal titration calorimetry, Int. J. Mol. Sci. 10, 3457-3477 (2009).

All documents cited in this application are hereby incorporated by reference as if recited in full herein.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

What is claimed is:

1. A method for preparing an aptamer for a target molecule, comprising the steps of:
(a) screening and selecting a candidate aptamer for the target molecule through standard selection protocols;
(b) modifying the candidate aptamer by having an N₂₂ random insertion to form a library used to identify a sequence that recognizes at least one functional group on the target molecule;
(c) generating a library of random 22-mers with the sequence identified in step (b) positioned next to the closing stem of the candidate aptamer;
(d) performing a functional-group binding free energy analysis for aptamers from the library generated in step (c); and
(e) identifying the aptamer that has the highest affinity for the target molecule.

2. The method of claim 1, wherein the target molecule is selected from the group consisting of: ammonia, glycinamide, methylamine, phenylethylamine, glycine, phenylalanine, methylbutylamine (1-Amino-3-methylbutane hydrochloride), tryptamine, methylene blue, histamine, serotonin, tryptophan, melatonin, L-DOPA (3,4-Dihydroxy-L-phenylalanine), norepinephrine, epinephrine, GABA (γ-Aminobutyric acid), GABA-amid (4-Aminobutyramide hydrochloride), glutamine, phenylalaninamide, tyrosinamide, leucine, tyrosine, tyramine, agmatine, arginine, dopamine, voriconazole, Cp*Rh(III) (pentamethylcyclo-pentadienylrhodium(II) chloride), Leu-Cp*Rh(iii), and Leu-Cu(ii).

3. The method of claim 1, wherein the target molecule is leucine or voriconazole.

4. An aptamer having the structure of:

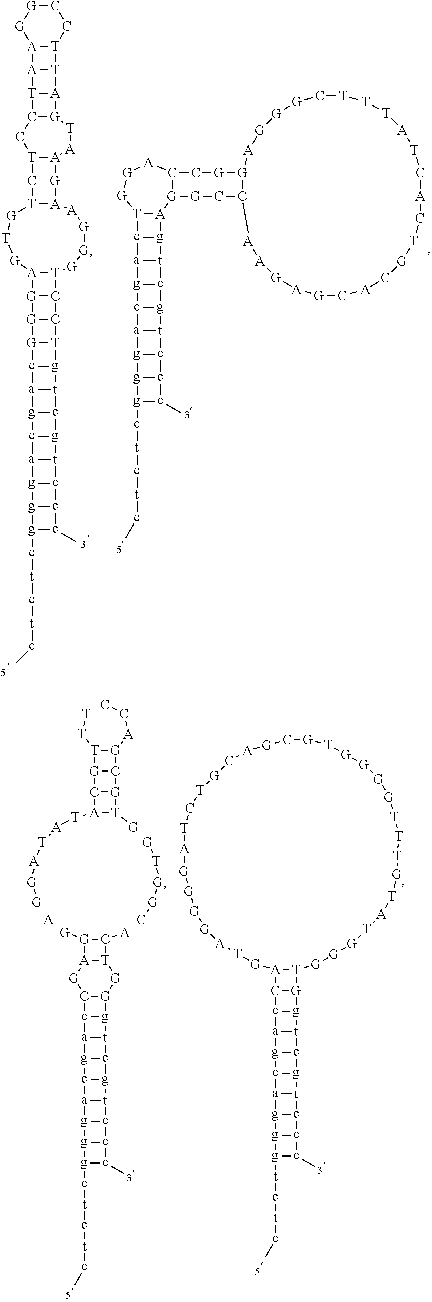

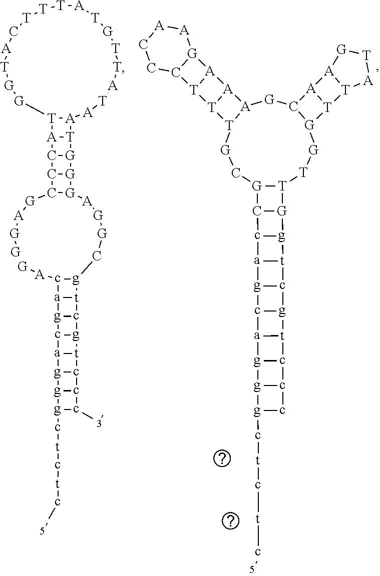

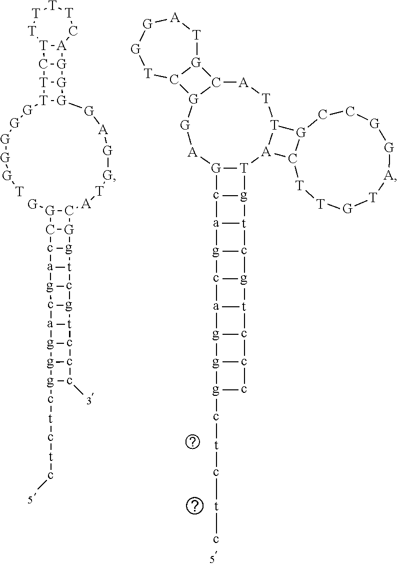

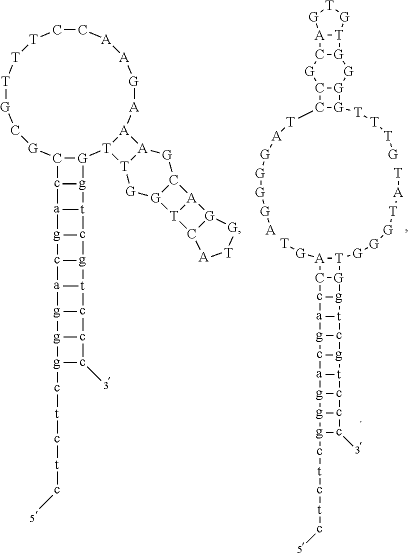

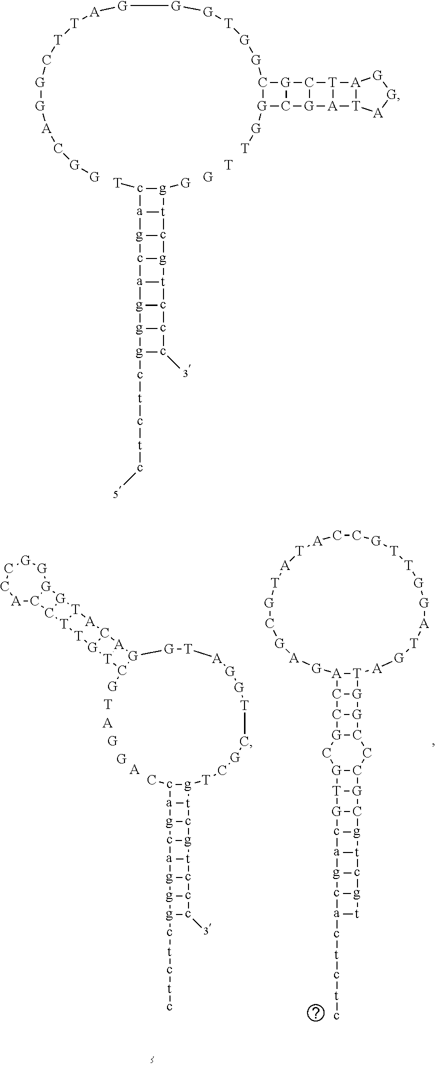

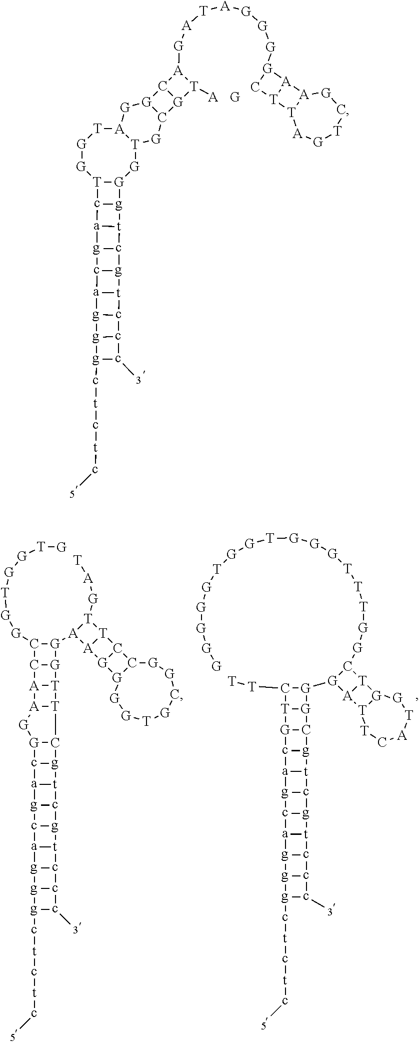

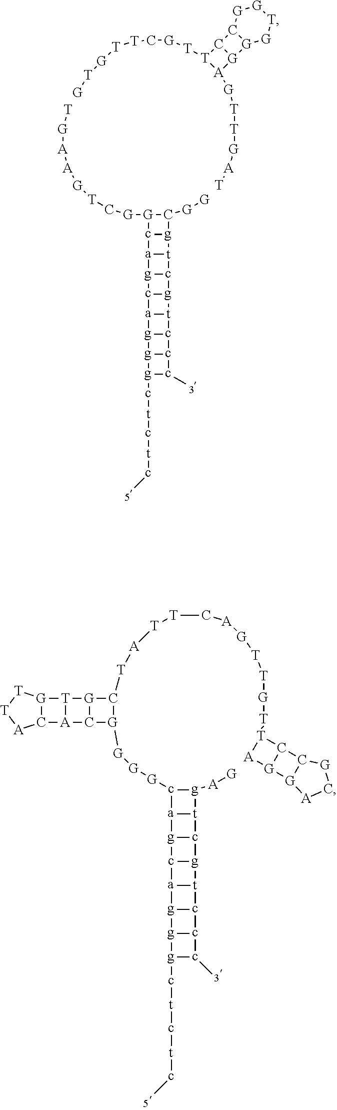

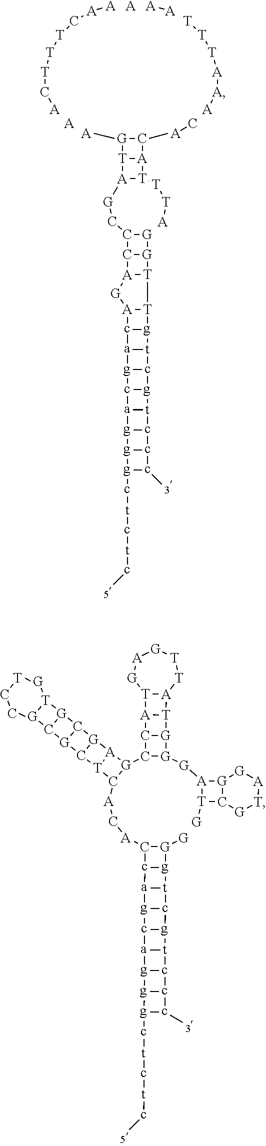

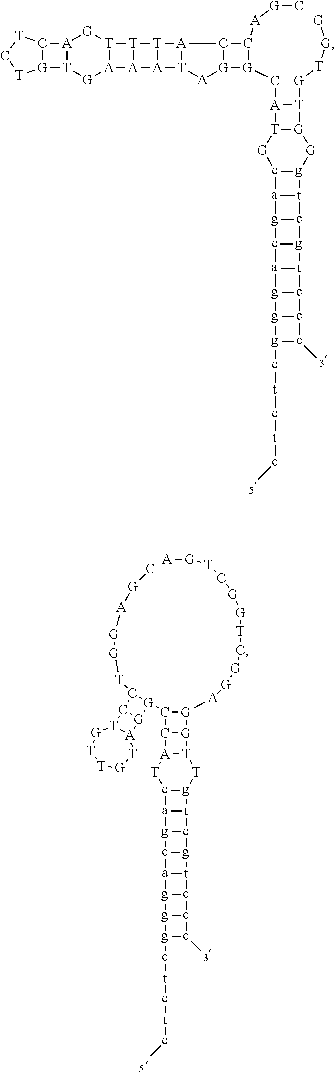

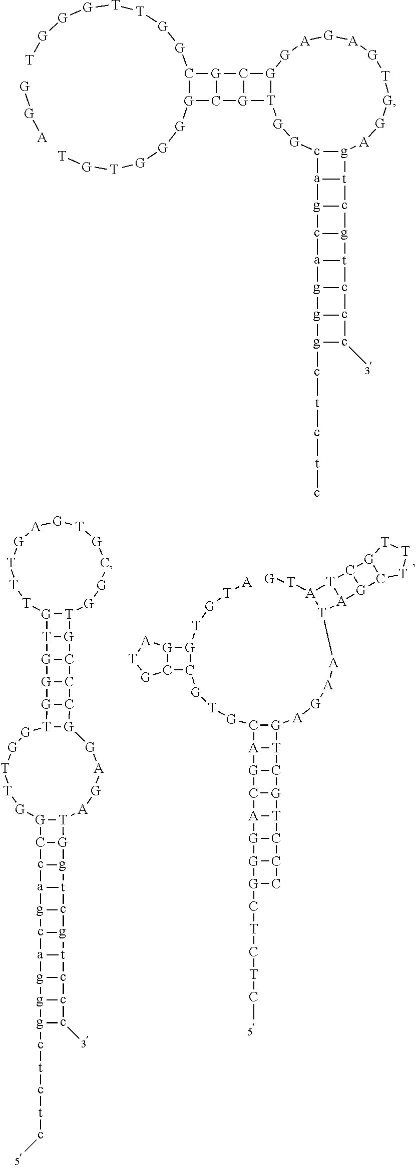

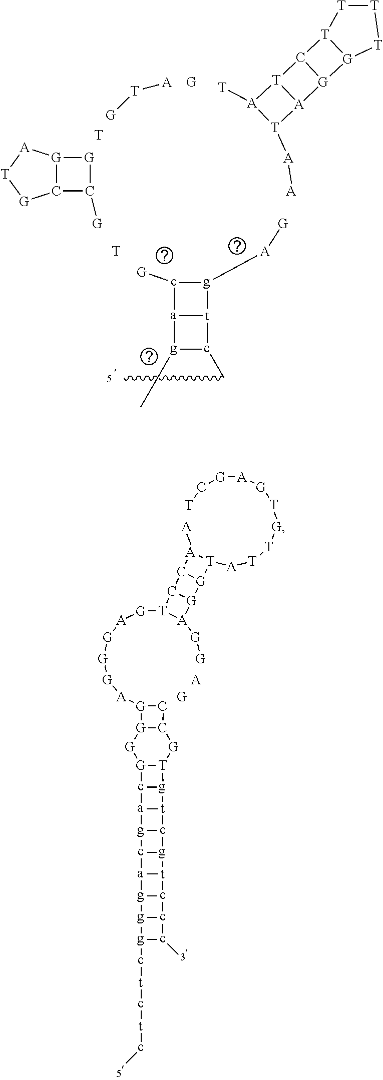

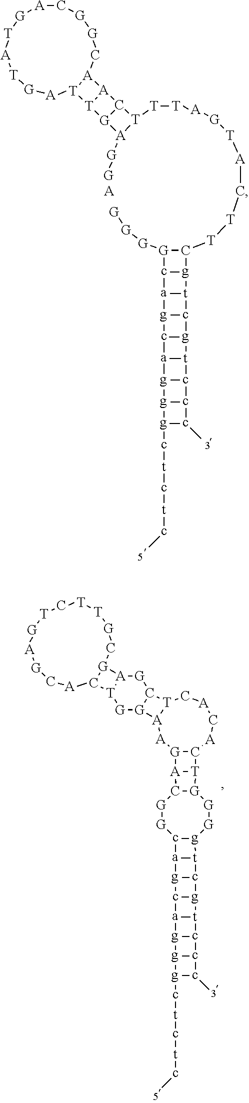

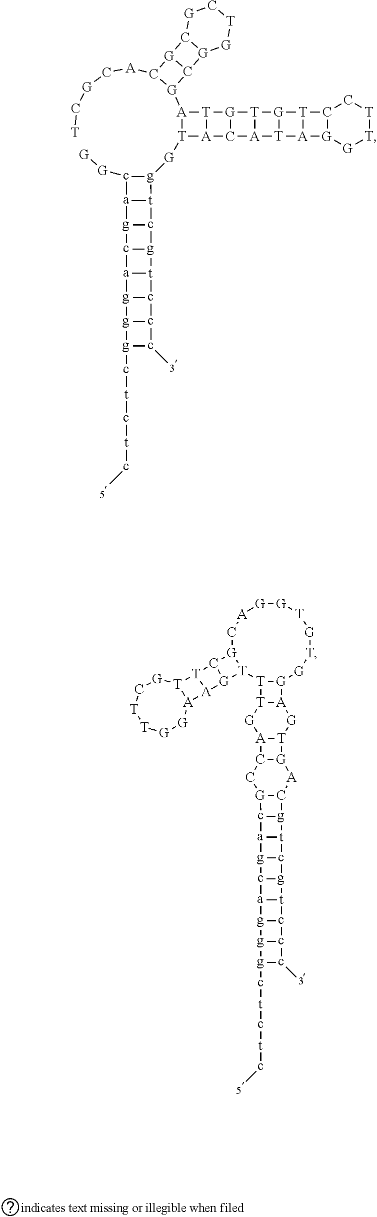

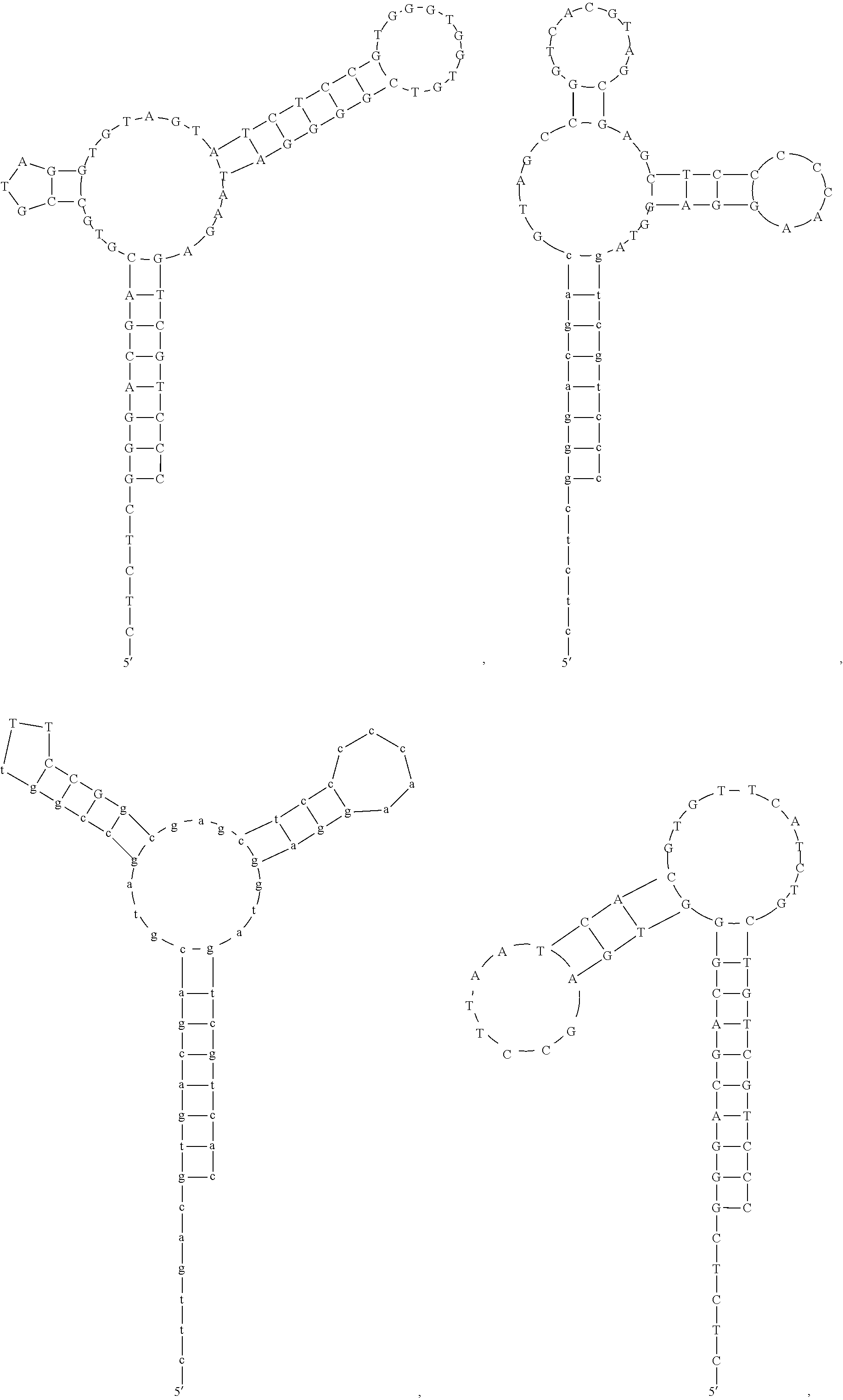

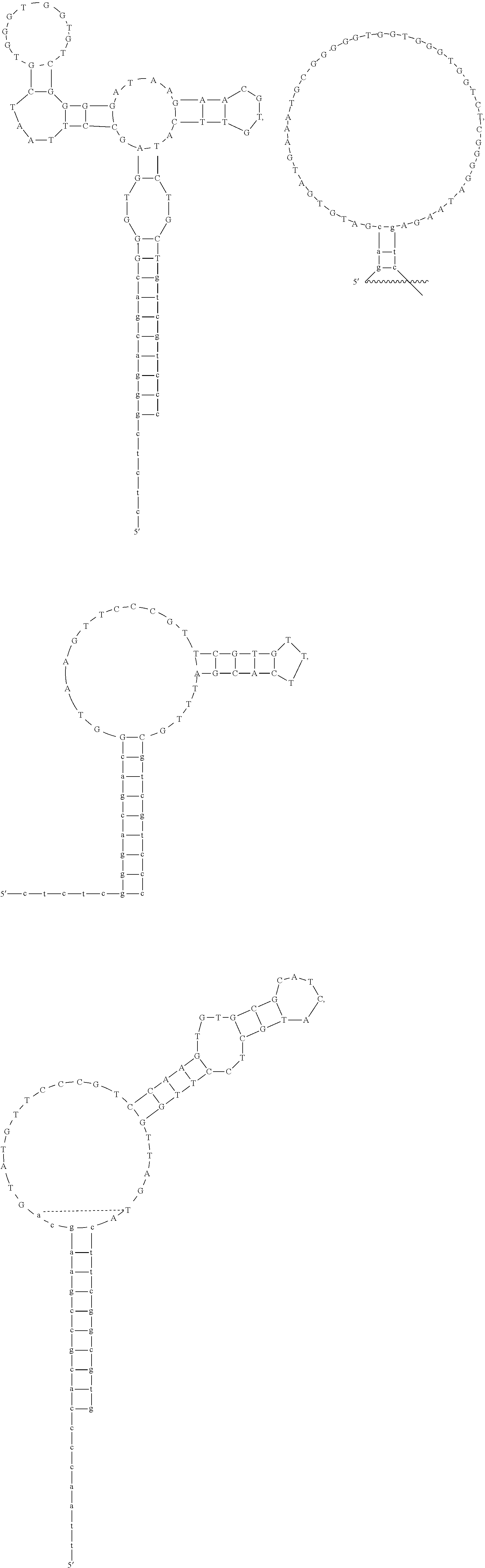

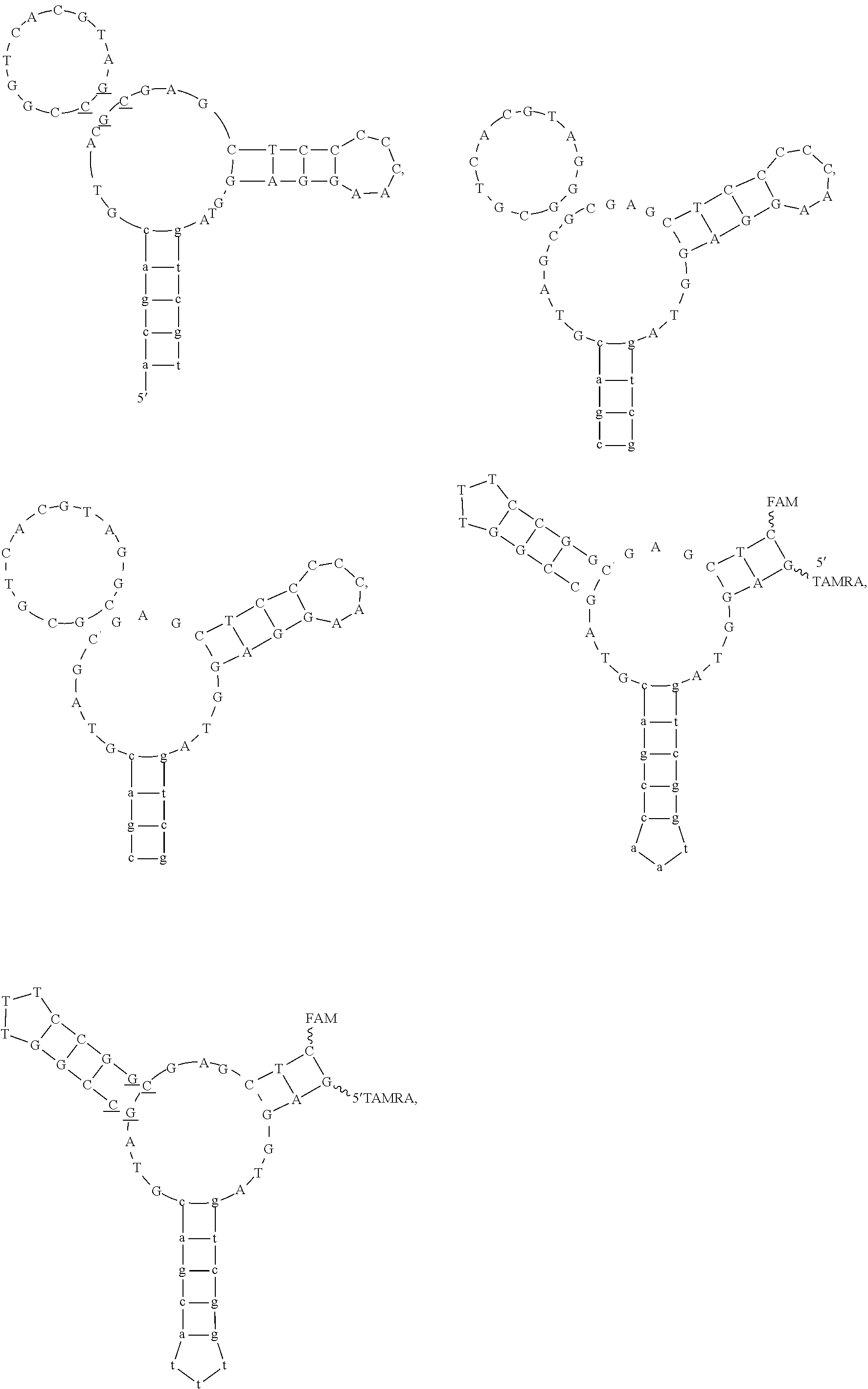

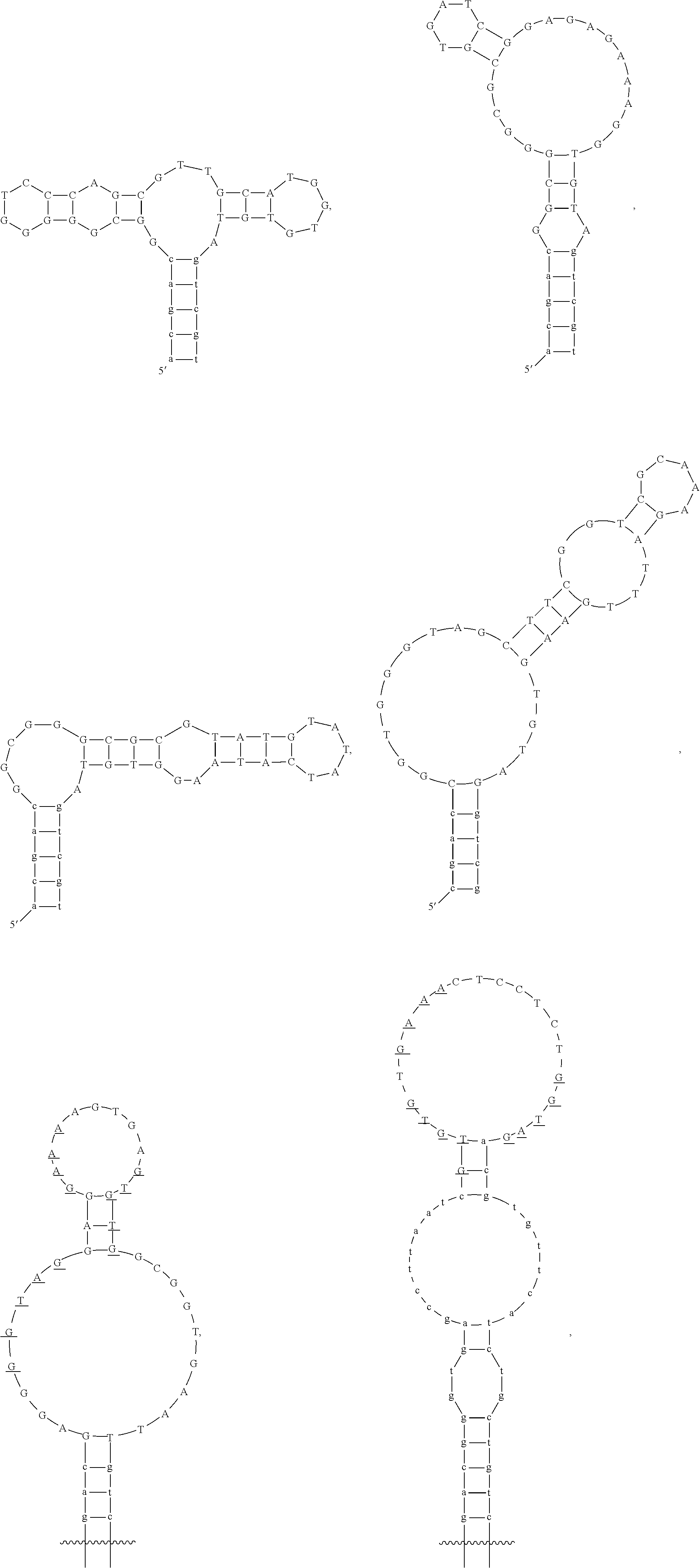

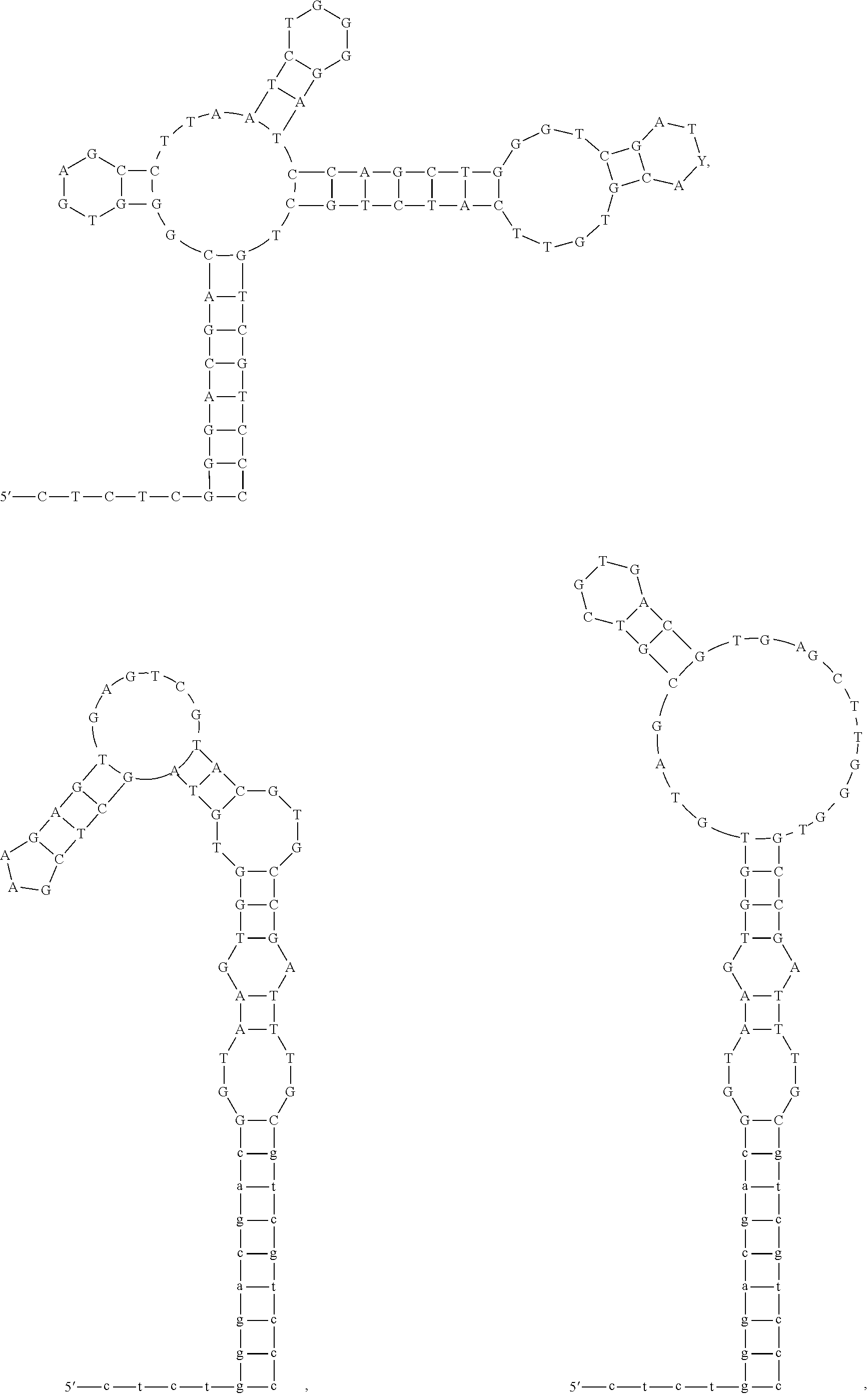

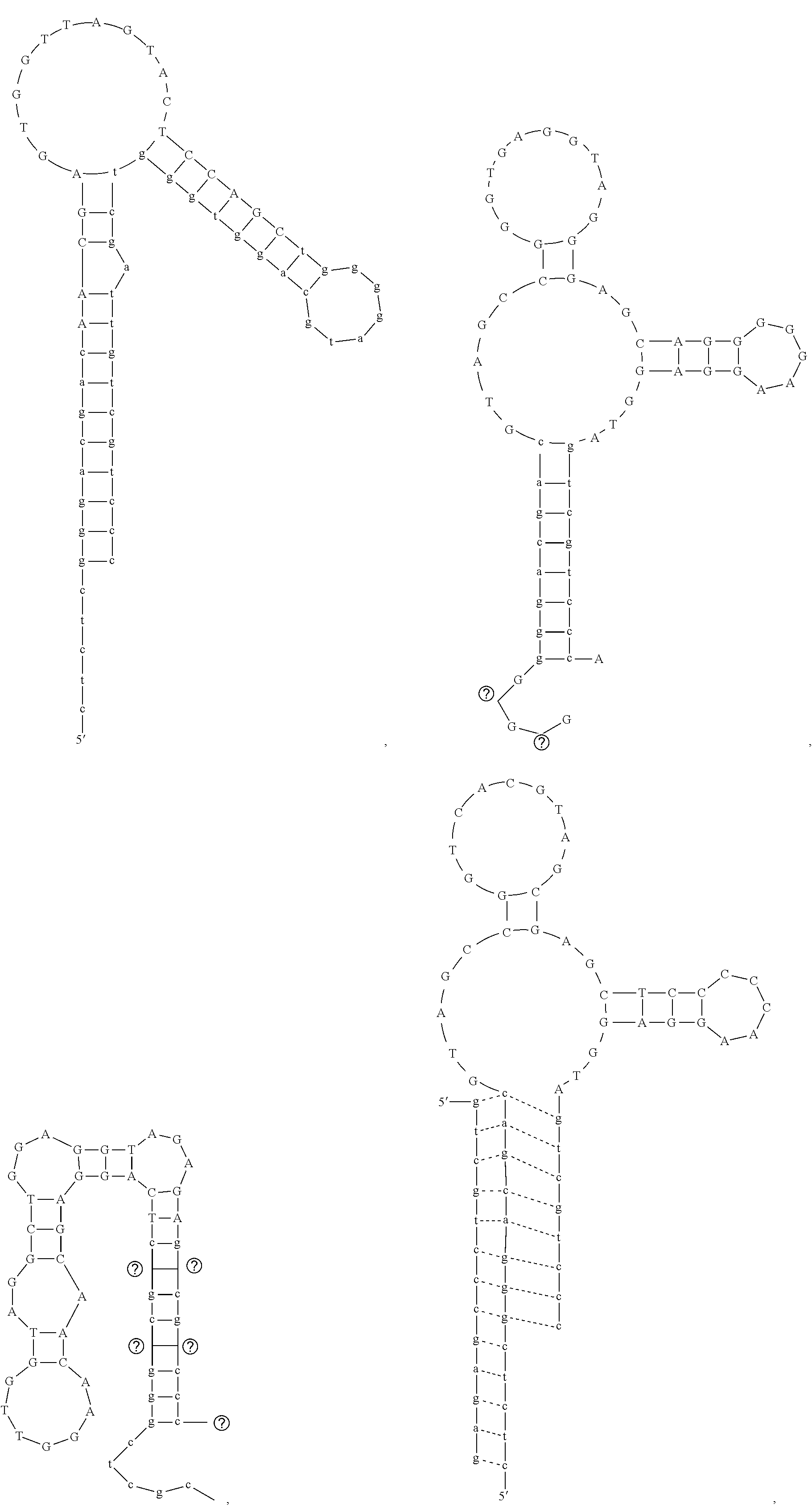

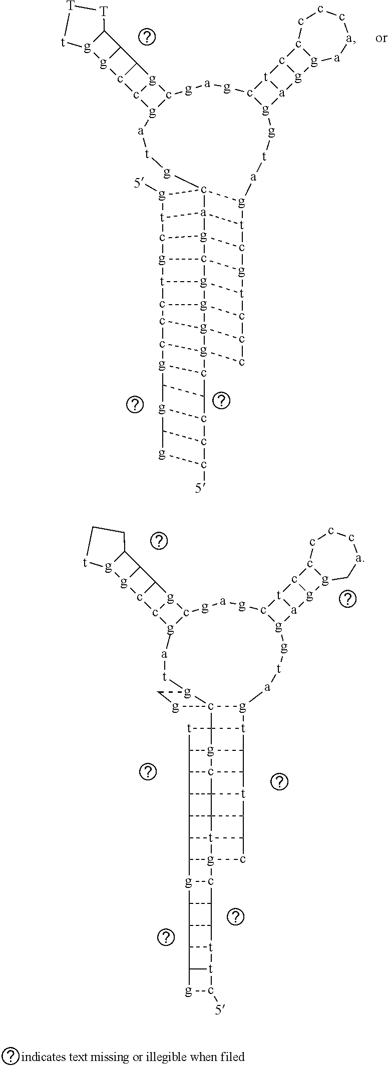

5. A composition comprising one or more aptamer prepared according to the method of claim 1, and a pharmaceutically acceptable carrier, adjuvant, or vehicle.

6. A composition comprising one or more aptamer according to claim 4, and a pharmaceutically acceptable carrier, adjuvant, or vehicle.

7. A method for treating or ameliorating the effects of a condition in a subject in need thereof, comprising administering to the subject an effective amount of one or more aptamer prepared according to the method of claim 1.

8. The method of claim 7, wherein the condition is selected from the group consisting of Age-related macular degeneration, myasthenia gravis, Acute myeloid leukaemia, Percutaneous coronary intervention, Thrombotic microangiopathies and carotid artery disease, Cardiopulmonary bypass to maintain steady state of anticoagulation, Type 2 diabetes, diabetic nephropathy, Multiple myeloma and non-Hodgkin's lymphoma, chronic inflammatory disease, progressive malignant prostate disease, viral infection, lupus, migraine, epithelial hyperproliferative disease, septic shock, Acute respiratory distress syndrome (ARDS), and combinations thereof.

9. The method of claim 7, wherein the subject is selected from the group consisting of humans, veterinary animals, and agricultural animals.

10. The method of claim 7, wherein the subject is a human.

11. A method for treating or ameliorating the effects of a condition in a subject in need thereof, comprising administering to the subject an effective amount of one or more aptamer according to claim 4.

12. A method for treating or ameliorating the effects of a condition in a subject in need thereof, comprising administering to the subject an effective amount of the composition of claim 5.

13. A method for treating or ameliorating the effects of a condition in a subject in need thereof, comprising administering to the subject an effective amount of the composition of claim 6.

14. A kit for treating or ameliorating the effects of a condition in a subject in need thereof, comprising an effective amount of one or more aptamer prepared according to the method of claim 1, packaged with its instructions for use.

15. A kit for treating or ameliorating the effects of a condition in a subject in need thereof, comprising an effective amount of one or more aptamer according to claim 4, packaged with its instructions for use.

16. A kit treating or ameliorating the effects of a condition in a subject in need thereof, comprising an effective amount of the composition of claim 5, packaged with its instructions for use.

17. A kit treating or ameliorating the effects of a condition in a subject in need thereof, comprising an effective amount of the composition of claim 6, packaged with its instructions for use.


SEQUENCE LISTING (163 sequences)

SEQ ID NO: 1 - DNA, 73 bp, synthetic construct
  source 1..73: mol_type = other DNA
ggaggctctcgggacgacnnnnnnnnnnnnnnnnnnnnnnnnnnnnnngtcgtcccgcctttaggatttacag

SEQ ID NO: 2 - DNA, 18 bp, synthetic construct
  source 1..18: mol_type = other DNA
gtcgtcccgagagccata

SEQ ID NO: 3 - DNA, 18 bp, synthetic construct
  source 1..18: mol_type = other DNA
ggaggctctcgggacgac

SEQ ID NO: 4 - DNA, 25 bp, synthetic construct
  source 1..25: mol_type = other DNA
ctgtaaatcctaaaggcgggacgac

SEQ ID NO: 5 - DNA, 52 bp, synthetic construct
  source 1..52: mol_type = other DNA
gaacaaccgaaaaaaggggaacccaagacccaaaggaggctctcgggacgac

SEQ ID NO: 6 - DNA, 53 bp, synthetic construct
  source 1..53: mol_type = other DNA
ggaaaaacccaaggaccaaactgtaaacctgtaaatcctaaaggcgggacgac

SEQ ID NO: 7 - DNA, 87 bp, synthetic construct
  source 1..87: mol_type = other DNA
caatatgacctcactctcgggacgacnnnnnnnnnnnnnnnnnnnnnnnnnnnnnnnnnnnngtcgtccctactcaggtcacgcagc

SEQ ID NO: 8 - DNA, 18 bp, synthetic construct
  source 1..18: mol_type = other DNA
gtcgtcccgagagtgtaa

SEQ ID NO: 9 - DNA, 26 bp, synthetic construct
  source 1..26: mol_type = other DNA
caatatgacctcactctcgggacgac

SEQ ID NO: 10 - DNA, 25 bp, synthetic construct
  source 1..25: mol_type = other DNA
gctgcgtgacctgagtagggacgac

SEQ ID NO: 11 - DNA, 54 bp, synthetic construct
  source 1..54: mol_type = other DNA
gaacaaccgaaaaaaggggaacccaagacccaaacaatatgacctcactctcgg

SEQ ID NO: 12 - DNA, 57 bp, synthetic construct
  source 1..57: mol_type = other DNA
ggaaaaacccaaggaccaaactgtaaacctgtaaatcctgctgcgtgacctgagtag

SEQ ID NO: 13 - DNA, 73 bp, synthetic construct
  source 1..73: mol_type = other DNA
ctctcgggacgacgggtgagccttaatcnnnnnnnnnnnnnnnnnnnnnnacgtgttcatctgctgtcgtccc

SEQ ID NO: 14 - DNA, 18 bp, synthetic construct
  source 1..18: mol_type = other DNA
gtcgtcccgagagccata

SEQ ID NO: 15 - DNA, 28 bp, synthetic construct
  source 1..28: mol_type = other DNA
ctctcgggacgacgggtgagccttaatc

SEQ ID NO: 16 - DNA, 23 bp, synthetic construct
  source 1..23: mol_type = other DNA
gggacgacagcagatgaacacgt

SEQ ID NO: 17 - DNA, 57 bp, synthetic construct
  source 1..57: mol_type = other DNA
gaacaaccgaaaaaaggggaacccaagacccaaaggaggctctcgggacgacgggtg

SEQ ID NO: 18 - DNA, 57 bp, synthetic construct
  source 1..57: mol_type = other DNA
aggaccaaactgtaaacctgtaaatcctaaagtaaaggcgggacgacagcagatgaa

SEQ ID NO: 19 - DNA, 70 bp, synthetic construct
  source 1..70: mol_type = other DNA
ggaggctctcgggacgacnnnnnnnnnnnnnnnnnnnnnngtgggtggtgtcggggataagagtcgtccc

SEQ ID NO: 20 - DNA, 18 bp, synthetic construct
  source 1..18: mol_type = other DNA
gtcgtcccgagagccata

SEQ ID NO: 21 - DNA, 18 bp, synthetic construct
  source 1..18: mol_type = other DNA
ggaggctctcgggacgac

SEQ ID NO: 22 - DNA, 30 bp, synthetic construct
  source 1..30: mol_type = other DNA
gggacgactcttatccccgacaccacccac

SEQ ID NO: 23 - DNA, 52 bp, synthetic construct; identical to SEQ ID NO: 5
  source 1..52: mol_type = other DNA

SEQ ID NO: 24 - DNA, 63 bp, synthetic construct
  source 1..63: mol_type = other DNA
ggaaaaacccaaggaccaaactgtaaacctgtaaatcctaaaggcgggacgactcttatcccc

SEQ ID NO: 25 - DNA, 70 bp, synthetic construct
  source 1..70: mol_type = other DNA
ggaggctctcgggacgacnnnnnnnnnnnnnnnnnnnnnntggggatgcaggtgggtcgattgtcgtccc

SEQ ID NO: 26 - DNA, 18 bp, synthetic construct
  source 1..18: mol_type = other DNA
gtcgtcccgagagccata

SEQ ID NO: 27 - DNA, 18 bp, synthetic construct
  source 1..18: mol_type = other DNA
ggaggctctcgggacgac

SEQ ID NO: 28 - DNA, 30 bp, synthetic construct
  source 1..30: mol_type = other DNA
gggacgacaatcgacccacctgcatcccca

SEQ ID NO: 29 - DNA, 52 bp, synthetic construct; identical to SEQ ID NO: 5
  source 1..52: mol_type = other DNA

SEQ ID NO: 30 - DNA, 63 bp, synthetic construct
  source 1..63: mol_type = other DNA
ggaaaaacccaaggaccaaactgtaaacctgtaaatcctaaaggcgggacgacaatcgaccca

SEQ ID NO: 31 - DNA, 72 bp, synthetic construct
  source 1..72: mol_type = other DNA
ggaggctctcgggacgacnnnnnnnnnnnnnnnnnnnnnnnnnnnnnngtcgtcccgatgctgcaatcgtaa

SEQ ID NO: 32 - DNA, 40 bp, synthetic construct
  source 1..40: mol_type = other DNA
acgacggcgggggtcccagcgttgcatggtgtgtagtcgt

SEQ ID NO: 33 - DNA, 40 bp, synthetic construct
  source 1..40: mol_type = other DNA
acgacggcgggcgcgtgatcggagagaaaggtgtagtcgt

SEQ ID NO: 34 - DNA, 40 bp, synthetic construct
  source 1..40: mol_type = other DNA
acgacggcgggcgcgtatgtatatcataaggtgtagtcgt

SEQ ID NO: 35 - DNA, 44 bp, synthetic construct
  source 1..44: mol_type = other DNA
cgaccggtgggtagcttcggtcgcaagatttgaagtgtaggtcg

SEQ ID NO: 36 - DNA, 57 bp, synthetic construct
  source 1..57: mol_type = other DNA
ctctcgggacgacnnnnnnnnnnnnnnnnnnnnnnnnnnnnnnnnnnnngtcgtccc

SEQ ID NO: 37 - DNA, 57 bp, synthetic construct
  source 1..57: mol_type = other DNA
ctctcgggacgaccgtttgaactcaattaggttgcatcggtcccacgaggtcgtccc

SEQ ID NO: 38 - DNA, 57 bp, synthetic construct
  source 1..57: mol_type = other DNA
ctctcgggacgacggtaagttcccgttcatgttgtccatcatgatttgcgtcgtccc

SEQ ID NO: 39 - DNA, 57 bp, synthetic construct
  source 1..57: mol_type = other DNA
ctctcgggacgaccagtttggcgttttgtacgcgcatcggtcccacgtggtcgtccc

SEQ ID NO: 40 - DNA, 57 bp, synthetic construct
  source 1..57: mol_type = other DNA
ctctcgggacgacggccccggggcaaacaacagcataggttgtttacgagtcgtccc

SEQ ID NO: 41 - DNA, 58 bp, synthetic construct
  source 1..58: mol_type = other DNA
ctctcgggacgacgggcaatttgtagcctgatagtaagttcccgttgaacgtcgtccc

SEQ ID NO: 42 - DNA, 57 bp, synthetic construct
  source 1..57: mol_type = other DNA
ctctcgggacgacgggtaagttcccgtacgctatacataacgttttgtcgtcgtccc

SEQ ID NO: 43 - DNA, 57 bp, synthetic construct
  source 1..57: mol_type = other DNA
ctctcgggacgacgagaaaagggttgatcaatactattggaatgtcgcagtcgtccc

SEQ ID NO: 44 - DNA, 57 bp, synthetic construct
  source 1..57: mol_type = other DNA
ctctcgggacgacggccaatgggagaaattaggggtagccgcctgggttgtcgtccc

SEQ ID NO: 45 - DNA, 57 bp, synthetic construct
  source 1..57: mol_type = other DNA
ctctcgggacgaccgcctgactacaagtggtagacatgggagcaattaggtcgtccc

SEQ ID NO: 46 - DNA, 57 bp, synthetic construct
  source 1..57: mol_type = other DNA
ctctcgggacgacggttccagggcaccaagatgattaatcgtggtacgagtcgtccc

SEQ ID NO: 47 - DNA, 57 bp, synthetic construct
  source 1..57: mol_type = other DNA
ctctcgggacgaccagtaagttcccgtacgcatttggttagtacttgtggtcgtccc

SEQ ID NO: 48 - DNA, 57 bp, synthetic construct
  source 1..57: mol_type = other DNA
ctctcgggacgacggagtgtgggagaaattagcatacagcgcccaggatgtcgtccc

SEQ ID NO: 49 - DNA, 57 bp, synthetic construct
  source 1..57: mol_type = other DNA
ctctcgggacgaccatttgtcctgtgaacataggagtaagtgcccgttggtcgtccc

SEQ ID NO: 50 - DNA, 57 bp, synthetic construct
  source 1..57: mol_type = other DNA
ctctcgggacgacgggagtgtctcctaaggccttagtaagaagggtcctgtcgtccc

SEQ ID NO: 51 - DNA, 13 bp, synthetic construct
  source 1..13: mol_type = other DNA
gtcgtcccgagag

SEQ ID NO: 52 - DNA, 38 bp, synthetic construct
  source 1..38: mol_type = other DNA
cgggagtgtctcctaaggccttagtaagaagggtcctg

SEQ ID NO: 53 - DNA, 57 bp, synthetic construct
  source 1..57: mol_type = other DNA
ctctcgggacgactggaccggagggctttatcactgcacgagaaccggagtcgtccc

SEQ ID NO: 54 - DNA, 13 bp, synthetic construct
  source 1..13: mol_type = other DNA
gtcgtcccgagag

SEQ ID NO: 55 - DNA, 46 bp, synthetic construct
  source 1..46: mol_type = other DNA
acgactggaccggagggctttatcactgcacgagaaccggagtcgt

SEQ ID NO: 56 - DNA, 57 bp, synthetic construct
  source 1..57: mol_type = other DNA
ctctcgggacgaccgaggaggatatacgtttccagcgtggtggcactgggtcgtccc

SEQ ID NO: 57 - DNA, 14 bp, synthetic construct
  source 1..14: mol_type = other DNA
ggtcgtcccgagag

SEQ ID NO: 58 - DNA, 44 bp, synthetic construct
  source 1..44: mol_type = other DNA
cgaccgaggaggatatacgtttccagcgtggtggcactgggtcg

SEQ ID NO: 59 - DNA, 57 bp, synthetic construct
  source 1..57: mol_type = other DNA
ctctcgggacgaccagtaggggatctgcagcgtggggtttgtatgggtggtcgtccc

SEQ ID NO: 60 - DNA, 14 bp, synthetic construct
  source 1..14: mol_type = other DNA
ggtcgtcccgagag

SEQ ID NO: 61 - DNA, 42 bp, synthetic construct
  source 1..42: mol_type = other DNA
gaccagtaggggatctgcagcgtggggtttgtatgggtggtc

SEQ ID NO: 62 - DNA, 56 bp, synthetic construct
  source 1..56: mol_type = other DNA
ctctcgggacgacagggagcccatggtactttatgttatmtgggaggcgtcgtccc

SEQ ID NO: 63 - DNA, 13 bp, synthetic construct
  source 1..13: mol_type = other DNA
gtcgtcccgagag

SEQ ID NO: 64 - DNA, 45 bp, synthetic construct
  source 1..45: mol_type = other DNA
acgacagggagcccatggtactttatgttatmtgggaggcgtcgt

SEQ ID NO: 65 - DNA, 51 bp, synthetic construct
  source 1..51: mol_type = other DNA
ctctcgggacgaccgcgtttcccaagaaagcaagtattggttggtcgtccc

SEQ ID NO: 66 - DNA, 14 bp, synthetic construct
  source 1..14: mol_type = other DNA
ggtcgtcccgagag

SEQ ID NO: 67 - DNA, 36 bp, synthetic construct
  source 1..36: mol_type = other DNA
gagcgcgtttcccaagaaagcaagtattggttggtg

SEQ ID NO: 68 - DNA, 51 bp, synthetic construct
  source 1..51: mol_type = other DNA
ctctcgggacgaccggtgggggttctttttcaggggaggtacggtcgtccc

SEQ ID NO: 69 - DNA, 13 bp, synthetic construct
  source 1..13: mol_type = other DNA
gtcgtcccgagag

SEQ ID NO: 70 - DNA, 36 bp, synthetic construct
  source 1..36: mol_type = other DNA
gaccggtgggggttctttttcaggggaggtacggtc

SEQ ID NO: 71 - DNA, 51 bp, synthetic construct
  source 1..51: mol_type = other DNA
ctctcgggacgacgaggctggatgcattcgccggatgttcgatgtcgtccc

SEQ ID NO: 72 - DNA, 13 bp, synthetic construct
  source 1..13: mol_type = other DNA
gtcgtcccgagag

SEQ ID NO: 73 - DNA, 38 bp, synthetic construct
  source 1..38: mol_type = other DNA
cgacgaggctggatgcattcgccggatgttcgatgtcg

SEQ ID NO: 74 - DNA, 50 bp, synthetic construct
  source 1..50: mol_type = other DNA
ctctcgggacgaccgcgtttccaagaaagcaggtactggttggtcgtccc

SEQ ID NO: 75 - DNA, 14 bp, synthetic construct
  source 1..14: mol_type = other DNA
ggtcgtcccgagag

SEQ ID NO: 76 - DNA, 37 bp, synthetic construct
  source 1..37: mol_type = other DNA
cgaccgcgtttccaagaaagcaggtactggttggtcg

SEQ ID NO: 77 - DNA, 57 bp, synthetic construct
  source 1..57: mol_type = other DNA
ctctcgggacgaccagtaggggatccgcagtgtggggtttgtatgggtggtcgtccc

SEQ ID NO: 78 - DNA, 14 bp, synthetic construct
  source 1..14: mol_type = other DNA
ggtcgtcccgagag

SEQ ID NO: 79 - DNA, 44 bp, synthetic construct
  source 1..44: mol_type = other DNA
cgaccagtaggggatccgcagtgtggggtttgtatgggtggtcg

SEQ ID NO: 80 - DNA, 56 bp, synthetic construct
  source 1..56: mol_type = other DNA
ctctcgggacgactggcaggcttagggtggcgctaggatagcggttgggtcgtccc

SEQ ID NO: 81 - DNA, 13 bp, synthetic construct
  source 1..13: mol_type = other DNA
gtcgtcccgagag

SEQ ID NO: 82 - DNA, 45 bp, synthetic construct
  source 1..45: mol_type = other DNA
acgactggcaggcttagggtggcgctaggatagcggttgggtcgt

SEQ ID NO: 83 - DNA, 57 bp, synthetic construct
  source 1..57: mol_type = other DNA
ctctcgggacgaccaggatgctgttccaccggggtacaggtaggtcgctgtcgtccc

SEQ ID NO: 84 - DNA, 13 bp, synthetic construct
  source 1..13: mol_type = other DNA
gtcgtcccgagag

SEQ ID NO: 85 - DNA, 48 bp, synthetic construct
  source 1..48: mol_type = other DNA
gacgaccaggatgctgttccaccggggtacaggtaggtcgctgtcgtc

SEQ ID NO: 86 - DNA, 51 bp, synthetic construct
  source 1..51: mol_type = other DNA
ctctcacgacgtgcgccagagcgtataccgttggatgatggcccgcgtcgt

SEQ ID NO: 87 - DNA, 13 bp, synthetic construct
  source 1..13: mol_type = other DNA
cacgtcgtgagag

SEQ ID NO: 88 - DNA, 40 bp, synthetic construct
  source 1..40: mol_type = other DNA
acgtgcgccagagcgtataccgttggatgatggcccgcgt

SEQ ID NO: 89 - DNA, 57 bp, synthetic construct
  source 1..57: mol_type = other DNA
ctctcgggacgactggtaggcagataggggaagctgattcgatgcgtgggtcgtccc

SEQ ID NO: 90 - DNA, 13 bp, synthetic construct
  source 1..13: mol_type = other DNA
gtcgtcccgagag

SEQ ID NO: 91 - DNA, 44 bp, synthetic construct
  source 1..44: mol_type = other DNA
cgactggtaggcagataggggaagctgattcgatgcgtgggtcg

SEQ ID NO: 92 - DNA, 57 bp, synthetic construct
  source 1..57: mol_type = other DNA
ctctcgggacgacggaaccggtggtgtagttccggcgtggggaaggttcgtcgtccc

SEQ ID NO: 93 - DNA, 14 bp, synthetic construct
  source 1..14: mol_type = other DNA
cgtcgtcccgagag

SEQ ID NO: 94 - DNA, 44 bp, synthetic construct
  source 1..44: mol_type = other DNA
cgacggaaccggtggtgtagttccggcgtggggaaggttcgtcg

SEQ ID NO: 95 - DNA, 57 bp, synthetic construct
  source 1..57: mol_type = other DNA
ctctcgggacgacgtcttgggggtggtgggtttggctggtacttagggcgtcgtccc

SEQ ID NO: 96 - DNA, 14 bp, synthetic construct
  source 1..14: mol_type = other DNA
cgtcgtcccgagag

SEQ ID NO: 97 - DNA, 40 bp, synthetic construct
  source 1..40: mol_type = other DNA
acgtcttgggggtggtgggtttggctggtacttagggcgt

SEQ ID NO: 98 - DNA, 57 bp, synthetic construct
  source 1..57: mol_type = other DNA
ctctcgggacgacggctgaagtgtgttcgttccggtgggagttgatggcgtcgtccc

SEQ ID NO: 99 - DNA, 15 bp, synthetic construct
  source 1..15: mol_type = other DNA
ccgtcgtcccgagag

SEQ ID NO: 100 - DNA, 46 bp, synthetic construct
  source 1..46: mol_type = other DNA
acgacggctgaagtgtgttcgttccggtgggagttgatggcgtcgt

SEQ ID NO: 101 - DNA, 57 bp, synthetic construct
  source 1..57: mol_type = other DNA
ctctcgggacgacggggcacattgtgctattcagttgttccgcaggagagtcgtccc

SEQ ID NO: 102 - DNA, 13 bp, synthetic construct
  source 1..13: mol_type = other DNA
gtcgtcccgagag

SEQ ID NO: 103 - DNA, 46 bp, synthetic construct
  source 1..46: mol_type = other DNA
acgacggggcacattgtgctattcagttgttccgcaggagagtcgt

SEQ ID NO: 104 - DNA, 62 bp, synthetic construct
  source 1..62: mol_type = other DNA
ctctcgggacgacagaccggatgaaactttcaaaaatttaaacacatttaggttgtcgtccc

SEQ ID NO: 105 - DNA, 14 bp, synthetic construct
  source 1..14: mol_type = other DNA
tgtcgtcccgagag

SEQ ID NO: 106 - DNA, 49 bp, synthetic construct
  source 1..49: mol_type = other DNA
cgacagaccggatgaaactttcaaaaatttaaacacatttaggttgtcg

SEQ ID NO: 107 - DNA, 66 bp, synthetic construct
  source 1..66: mol_type = other DNA
ctctcgggacgaccacactcgcgcctgtgcgagccatgagttatgggaggatgctggggtcgtccc

SEQ ID NO: 108 - DNA, 14 bp, synthetic construct
  source 1..14: mol_type = other DNA
ggtcgtcccgagag

SEQ ID NO: 109 - DNA, 53 bp, synthetic construct
  source 1..53: mol_type = other DNA
cgaccacactcgcgcctgtgcgagccatgagttatgggaggatgctggggtcg

SEQ ID NO: 110 - DNA, 57 bp, synthetic construct
  source 1..57: mol_type = other DNA
ctctcgggacgacgtacggataaagtgtctcagtttaccagcggtgtgggtcgtccc

SEQ ID NO: 111 - DNA, 14 bp, synthetic construct
  source 1..14: mol_type = other DNA
cgtcgtcccgagag

SEQ ID NO: 112 - DNA, 46 bp, synthetic construct
  source 1..46: mol_type = other DNA
acgacgtacggataaagtgtctcagtttaccagcggtgtgggtcgt

SEQ ID NO: 113 - DNA, 57 bp, synthetic construct
  source 1..57: mol_type = other DNA
ctctcgggacgactaccggatgttgtcctggagcagtcggtcggaggttgtcgtccc

SEQ ID NO: 114 - DNA, 14 bp, synthetic construct
  source 1..14: mol_type = other DNA
ggtcgtcccgagag

SEQ ID NO: 115 - DNA, 46 bp, synthetic construct
  source 1..46: mol_type = other DNA
acgactaccggatgttgtcctggagcagtcggtcggaggttgtcgt

SEQ ID NO: 116 - DNA, 57 bp, synthetic construct
  source 1..57: mol_type = other DNA
ctctcgggacgacggtgcggggtgtaggtgggttggcgcggagagtggagtcgtccc

SEQ ID NO: 117 - DNA, 14 bp, synthetic construct
  source 1..14: mol_type = other DNA
cgtcgtcccgagag

SEQ ID NO: 118 - DNA, 46 bp, synthetic construct
  source 1..46: mol_type = other DNA
acgacggtgcggggtgtaggtgggttggcgcggagagtggagtcgt

SEQ ID NO: 119 - DNA, 56 bp, synthetic construct
  source 1..56: mol_type = other DNA
ctctcgggacgaccggnggtgggtgtttgagtgcggtgcccggagatggtcgtccc

SEQ ID NO: 120 - DNA, 14 bp, synthetic construct
  source 1..14: mol_type = other DNA
ggtcgtcccgagag

SEQ ID NO: 121 - DNA, 42 bp, synthetic construct
  source 1..42: mol_type = other DNA
gaccggttggtgggtgtttgagtgcggtgcccggagatggtc

SEQ ID NO: 122 - DNA, 52 bp, synthetic construct
  source 1..52: mol_type = other DNA
ctctcgggacgacgtgccgtaggtgtagtatcgtttcgataagagtcgtccc

SEQ ID NO: 123 - DNA, 14 bp, synthetic construct
  source 1..14: mol_type = other DNA
cgtcgtcccgagag

SEQ ID NO: 124 - DNA, 43 bp, synthetic construct
  source 1..43: mol_type = other DNA
gacgacgtgccgtaggtgtagtatcgtttcgataagagtcgtc

SEQ ID NO: 125 - DNA, 58 bp, synthetic construct
  source 1..58: mol_type = other DNA
ctctcgggacgacggggagggagtccaatcgagtgttatggaggagccgtgtcgtccc

SEQ ID NO: 126 - DNA, 13 bp, synthetic construct
  source 1..13: mol_type = other DNA
gtcgtcccgagag

SEQ ID NO: 127 - DNA, 47 bp, synthetic construct
  source 1..47: mol_type = other DNA
acgacggggagggagtccaatcgagtgttatggaggagccgtgtcgt

SEQ ID NO: 128 - DNA, 57 bp, synthetic construct
  source 1..57: mol_type = other DNA
ctctcgggacgacggggaggagttagtatgacggcaactttagtacttcgtcgtccc

SEQ ID NO: 129 - DNA, 13 bp, synthetic construct
  source 1..13: mol_type = other DNA
gtcgtcccgagag

SEQ ID NO: 130 - DNA, 44 bp, synthetic construct
  source 1..44: mol_type = other DNA
cgacggggaggagttagtatgacggcaactttagtacttcgtcg

SEQ ID NO: 131 - DNA, 57 bp, synthetic construct
  source 1..57: mol_type = other DNA
ctctcgggacgacggcagaaggtcacgagtcttgcgagctcacactggggtcgtccc

SEQ ID NO: 132 - DNA, 14 bp, synthetic construct
  source 1..14: mol_type = other DNA
cgtcgtcccgagag

SEQ ID NO: 133 - DNA, 46 bp, synthetic construct
  source 1..46: mol_type = other DNA
acgacggcagaaggtcacgagtcttgcgagctcacactggggtcgt

SEQ ID NO: 134 - DNA, 57 bp, synthetic construct
  source 1..57: mol_type = other DNA
ctctcgggacgacggtcggaggcggtggcgatgtgtccttggatacatggtcgtccc

SEQ ID NO: 135 - DNA, 13 bp, synthetic construct
  source 1..13: mol_type = other DNA
gtcgtcccgagag

SEQ ID NO: 136 - DNA, 46 bp, synthetic construct
  source 1..46: mol_type = other DNA
acgacggtcgcacgcgctggcgatgtgtccttggatacatggtcgt

SEQ ID NO: 137 - DNA, 57 bp, synthetic construct
  source 1..57: mol_type = other DNA
ctctcgggacgacgccagtttgaaggttcgttcgcaggtgtggagtgacgtcgtccc

SEQ ID NO: 138 - DNA, 14 bp, synthetic construct
  source 1..14: mol_type = other DNA
cgtcgtcccgagag

SEQ ID NO: 139 - DNA, 44 bp, synthetic construct
  source 1..44: mol_type = other DNA
cgacgccagtttgaaggttcgttcgcaggtgtggagtgacgtcg

SEQ ID NO: 140 - DNA, 65 bp, synthetic construct
  source 1..65: mol_type = other DNA
ctctcgggacgacgtgccgtaggtgtagtatctccgtgggtggtgtcggggataagagtcgtccc

SEQ ID NO: 141 - DNA, 14 bp, synthetic construct
  source 1..14: mol_type = other DNA
cgtcgtcccgagag

SEQ ID NO: 142 - DNA, 54 bp, synthetic construct
  source 1..54: mol_type = other DNA
acgacgtgccgtaggtgtagtatctccgtgggtggtgtcggggataagagtcgt

SEQ ID NO: 143 - DNA, 57 bp, synthetic construct
  source 1..57: mol_type = other DNA
ctctcgggacgacgtagccggtcacgtagcgagctcccccaaggaggtagtcgtccc

SEQ ID NO: 144 - DNA, 13 bp, synthetic construct
  source 1..13: mol_type = other DNA
gtcgtcccgagag

SEQ ID NO: 145 - DNA, 46 bp, synthetic construct
  source 1..46: mol_type = other DNA
acgacgtagccggtcacgtagcgagctcccccaaggaggtagtcgt

SEQ ID NO: 146 - DNA, 57 bp, synthetic construct
  source 1..57: mol_type = other DNA
cttgacgtgacgacgtagccggtttccggcgagctcccccaaggaggtagtcgtcac

SEQ ID NO: 147 - DNA, 15 bp, synthetic construct
  source 1..15: mol_type = other DNA
cgtcgtcacgtcaag

SEQ ID NO: 148 - DNA, 45 bp, synthetic construct
  source 1..45: mol_type = other DNA
acgacgtagccggtttccggcgagctcccccaaggaggtagtcgt

SEQ ID NO: 149 - DNA, 51 bp, synthetic construct
  source 1..51: mol_type = other DNA
ctctcgggacgacgggtgagccttaatcacgtgttcatctgctgtcgtccc

SEQ ID NO: 150 - DNA, 13 bp, synthetic construct
  source 1..13: mol_type = other DNA
gtcgtcccgagag

SEQ ID NO: 151 - DNA, 36 bp, synthetic construct
  source 1..36: mol_type = other DNA
gacgggtgagccttaatcacgtgttcatctgctgtc

SEQ ID NO: 152 - DNA, 73 bp, synthetic construct
  source 1..73: mol_type = other DNA
ctctcgggacgacgggtgagccttaatcgtgggtggtgtcggggataagaacgtgttcatctgctgtcgtccc

SEQ ID NO: 153 - DNA, 13 bp, synthetic construct
  source 1..13: mol_type = other DNA
gtcgtcccgagag

SEQ ID NO: 154 - DNA, 58 bp, synthetic construct
  source 1..58: mol_type = other DNA
gacgggtgagccttaatcgtgggtggtgtcggggataagaacgtgttcatctgctgtc

SEQ ID NO: 155 - DNA, 65 bp, synthetic construct
  source 1..65: mol_type = other DNA
ctctcgggacgacgatgtgatgaaatgcgggggtggtgggtggtgtcggggataagagtcgtccc

SEQ ID NO: 156 - DNA, 14 bp, synthetic construct
  source 1..14: mol_type = other DNA
cgtcgtcccgagag

SEQ ID NO: 157 - DNA, 54 bp, synthetic construct
  source 1..54: mol_type = other DNA
acgacgatgtgatgaartgcgggggtggtgggtggtgtcggggataagagtcgt

SEQ ID NO: 158 - DNA, 73 bp, synthetic construct
  source 1..73: mol_type = other DNA
ctctcgggacgacgggtgagccttaatctggggatgcaggtgggtcgattacgtgttcatctgctgtcgtccc

SEQ ID NO: 159 - DNA, 13 bp, synthetic construct
  source 1..13: mol_type = other DNA
gtcgtcccgagag

SEQ ID NO: 160 - DNA, 62 bp, synthetic construct
  source 1..62: mol_type = other DNA
acgacgggtgagccttaatctggggatgcaggtgggtcgattacgtgttcatctgctgtcgt

SEQ ID NO: 161 - DNA, 65 bp, synthetic construct
  source 1..65: mol_type = other DNA
ctctcgggacgacaacgagtggttagtactccagctggggatgcaggtgggtcgattgtcgtccc

SEQ ID NO: 162 - DNA, 14 bp, synthetic construct
  source 1..14: mol_type = other DNA
tgtcgtcccgagag

SEQ ID NO: 163 - DNA, 52 bp, synthetic construct
  source 1..52: mol_type = other DNA
cgacaacgagtggttagtactccagctggggatgcaggtgggtcgattgtcg